(19) United States
(12) Patent Application Publication
(10) Pub. No.: US 2005/0049594 A1
(43) Pub. Date: Mar. 3, 2005

(54) Dual locking plate and associated method

(76) Inventors: Michael A. Wack, Warsaw, IN (US); Pamela C. Guzman, Fort Wayne, IN (US); Dennis A. Stoller, Fort Wayne, IN (US); Christopher K. Bremer, Warsaw, IN (US); Rebecca D. Oberst, Fort Wayne, IN (US); Mark A. Fenton, North Manchester, IN (US); George J. Haidukewych, Tampa, FL (US); Lawrence B. Bone, Buffalo, NY (US); Roy W. Sanders, Tampa, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/675,107

(22) Filed: Sep. 30, 2003

Related U.S. Application Data
(63) Continuation-in-part of application No. 10/633,469, filed on Aug. 1, 2003.
(63) Continuation-in-part of application No. 10/100,387, filed on Mar. 18, 2002.
(60) Provisional application No. 60/285,462, filed on Apr. 20, 2001.

(51) Int. Cl. A61B 17/56

(52) U.S. Cl. 606/69

(57) ABSTRACT

A system for percutaneous fracture repair of a bone is provided. The system includes a plate having a first feature and a second feature. The first feature and the second feature are spaced apart from each other. The plate defines a longitudinal axis of the plate. The system also includes a first attachment component operably associated with the first feature. The first attachment component cooperates with the bone. The system also includes a second attachment component operably associated with the second feature. The second attachment component is percutaneously inserted into the second feature. The second attachment component is operably associated with the plate to provide a compressive force in the bone. The compressive force has a component of the force in the longitudinal axis. The second attachment component is adapted for cooperation with the bone.

### CROSS REFERENCE TO RELATED APPLICATIONS

This application is a Continuation-in-Part of attorney docket number DEP 673CIP filed Aug. 1, 2003, entitled POLYAXIAL LOCKING PLATE. DEP 673CIP is a Continuation-in-Part of U.S. patent application Ser. No. 10/100,387 filed Mar. 18, 2002, entitled POLYAXIAL LOCKING PLATE. U.S. patent Application Ser. No. 10/100,387 is a Utility Application based upon U.S. Provisional Patent Application, Ser. No. 60/285,462 filed Apr. 20, 2001, entitled POLYAXIAL LOCKING PLATE. DEP 673CIP filed Aug. 1, 2003, entitled POLYAXIAL LOCKING PLATE and U.S. patent application Ser. No. 10/100,387 filed Mar. 18, 2002 entitled POLYAXIAL LOCKING PLATE are incorporated by reference herein in their entirety.

### BACKGROUND OF THE INVENTION

The present invention relates to a bone locking plate, more particularly the present invention relates to a bone locking plate that includes an adjustable attachment component. Most particularly, the present invention relates to a bone locking plate that includes an attachment component whose angle relative to the locking plate may be manipulated during surgery so that an accompanying screw extends into the bone in a desirable orientation.

The skeletal system includes many long bones which extend from the human torso. These long bones include the femur, fibula, tibia, humerus, radius and ulna. These long bones are particularly exposed to trauma from accidents and as such often are fractured during such trauma and may be subject to complex devastating fractures.

Automobile accidents for instance are a common cause of trauma to long bones. In particular the femur and tibia frequently fracture when the area around the knee is subjected to a frontal automobile accident.

Often the distal and/or proximal portions of the long bone, for example, the femur and tibia are fractured into several components and must be re-attached.

Mechanical devices most commonly in the form of pins, plates and screws are commonly used to attach fractured long bones. The plates, pins and screws are typically made of a durable material compatible with the human anatomy, for example titanium, stainless steel or cobalt chrome. The plates are typically positioned longitudinally along the periphery of the long bone and have holes or openings through which screws may be inserted into the long bone transversely. Additionally, intramedullary nails or screws may be utilized to secure fractured components of a long bone, for example, to secure the head of a femur.

Fractures of long bones typically occur in high stress areas, for example, near the condyles or distal or proximal portions of the long bones. Such fractures in the distal or proximal condyle portions of the long bone may result in many individual fragments which must be reconnected. Optimally, the bone plates should be positioned adjacent to the distal or proximal portions of the long bones and permit the securing of these fragments.

More recently bone plates have been provided for long bones which have a profile which conforms to the distal or proximal portion of the long bone. For example such bone plates are available from DePuy ACE in the form of supra condylar plate systems. These plates have a contoured periphery to match the distal portion of a long bone, for example, a femur. These plates, however, include holes or opening through which transverse screws are used to secure the bone plate to the long bone. The openings in the bone plate provide thus for only one general orientation of the screw for attachment of the bone fragments, which is normally or perpendicularly to the bone plate. Thus often the optimum position of a screw may not be utilized as it does not conform to a position nominal or perpendicular to the bone plate.

Often with a fracture of condyles of the distal portion of a long bone the adjacent screws should be positioned and locked in a divergent direction diverging from the bone plate so that the distal condyles may be properly secured by the bone screw. Two dimensional bone plates do not provide for the optimum diverging orientation of the bone screws.

Recently DePuy Acromed, Inc. has developed locking plates, as disclosed in U.S. Pat. No. 5,954,722 to Bono, for use in spinal applications which include a pivotable bushing within the plate which bushing is internally threaded and mates with external threads on bone screws. This type of locking plate permits an orientation of the bone screw in a position other than normally with the bone plate while also permitting locking of the screw.

Proper securement of a bone plate to a bone is dependent on, among other things, the condition of the bone. For example, if the bone is severely fractured, the fasteners are preferably unlocking or not rigidly secured to the plate. By not locking the fastener to the plate, the fastener can be used to pull or draw the fragments of the fractured bone together to assist in blood flow and the healing of the fracture site. Such non-locking fasteners may include, for example, fasteners with cancellous threads to securely contain the fragments. Non-locking fasteners may also include a portion of the stem which is not threaded or be in the form of a lagging screw to assist in the drawing of bone fragments together. Further, the use of a non-locking fastener results in increased flexion on motion between the fasteners and the plate thereby increasing the stress or load on the fracture site. Such increase in fracture load or bracing of the stress adjacent to fracture site results in hypertrophy or the increase in size of the cortical bone due to the physical activity to accommodate the higher stress. Such a reaction to the increased stress at the fracture site is well borne out by Wolff's Law.

Locking fasteners, for example, locking screws, however, provide for a more rigid construction and may provide an alternate construction for a bone plate and may be used in bone of any quality. For example, if the bone of the patient is osteoporotic or has a thin cortical layer or an eggshell cortical layer, the increased stress due to flexion between the fasteners and the bone plate caused by movable or unlocked fasteners, may fracture the cortical bone and not support such a construction. Thus, for osteoporotic bone, the use of fasteners locked to the bone plate is preferred. While x-rays and other analytical tools may be utilized to determine the type of bone of the patient, the actual condition of the bone of the patient may not be fully determined until the fracture sight is exposed. Thus, there is a need to interoperatively provide a plate which may be selectively locked or unlocked with respect to its fasteners.

Occasionally, when a fastener is used to secure a bone plate, the fastener is screwed into osteoporotic or otherwise weak bone and the fastener may become stripped or not properly secured into the bone. The fastener may be removed and a different location or bone site may be necessary to secure the plate with the fastener.

Occasionally, a bone plate will lift up or separate from the bone. This is particularly a problem with the portion of the bone plate opposite the head or condylar portion of the bone plate. As the patient moves, for example, walks, the bone plate flexes and the portion of the bone plate moves toward and away from the bone. This motion may cause the plate to loosen from the bone.

Compression of the bone at the fracture site may be desired when using bone plates. Compression can be a useful procedure to pull larger fragments in line and to encourage a faster rate of healing. Compression is particularly well suited to correct fractures in which the fractures are highly comminuted or have a large number of fragments. The compression of the bone is typically accomplished by first securing the bone plate to a position spaced from the fracture site and compressing the bone as the plate is secured at a position spaced from the fracture site and opposed to the first anchored position. An open procedure is required for the use of compression with bone plates to permit access to the bone plate on both sides of the fracture site. The open procedure results in a large scar for the patient as well as creating an environment for an infection and creating a longer healing period.

Attempts have been made to implant bone plate percutaneously, or implant the bone plate with a minimal incision in the skin. Problems have occurred in properly and securely moving the bone plate adjacent the bone to percutaneously position it in the proper location.

### SUMMARY OF THE INVENTION

According to the present invention, a system for percutaneous fracture repair of a bone is provided. The system includes a plate having a first feature and a second feature. The first feature and the second feature are spaced apart from each other. The plate defines a longitudinal axis of the plate. The system also includes a first attachment component operably associated with the first feature. The first attachment component cooperates with the bone. The system also includes a second attachment component operably associated with the second feature. The second attachment component is percutaneously inserted into the second feature. The second attachment component is operably associated with the plate to provide a compressive force in the bone. The compressive force has a component of the force in the longitudinal axis. The second attachment component is adapted for cooperation with the bone.

According to the present invention, a system for percutaneous fracture repair of a long bone including a shaft portion and a condylar portion of the long bone is provided. The long bone defines a fracture of the long bone. The fracture is positioned at least partially between the shaft portion and the condylar portion. The system includes a plate having a first portion for cooperation with the condylar portion and a second portion for cooperation with the shaft portion. The first portion defines a first opening through the plate and the second portion defines a second opening through the plate. The first opening and the second opening are spaced apart from each other. The plate defines a longitudinal axis of the plate extending from the first portion to the second portion of the plate. The system also includes a first fastener adapted to at least partially pass through the first opening. The first fastener is adapted to at least partially engage with the condylar portion of the bone. The system also includes a second fastener adapted to at least partially pass through the second opening. The second fastener is adapted to at least partially engage with the shaft portion of the bone. The second fastener is percutaneously inserted into the second opening. The second fastener contacts the plate adjacent to the second opening of the plate to provide a compressive force in the bone. The compressive force has a component of the force in the longitudinal axis operably associated with the bone to provide a compressive force in the bone.

According to the present invention, a guide to assist in the percutaneous fracture repair of a bone having a first bone location and a spaced apart second bone location is provided. The guide is used to guide a fastener at least partially through an opening in a bone plate and into the bone. The guide includes a body attachable to the bone plate adjacent the first bone location and a tube. The tube is fitted to the body for guiding the fastener percutaneously at least partially through the bone plate opening in the bone plate and into the bone at the second bone location. The body and the tube are adapted to cooperate with the bone plate and with the fastener so that the bone is under compression between the first bone location and the second bone location.

Still further, in accordance with the present invention a method for repairing a bone fracture on a bone having a condylar portion and a shaft portion is provided. The method including the steps of providing a bone plate having a head portion for cooperation with the condylar portion and a body portion for cooperation with the shaft portion and a first opening in the head portion and a second opening in the body portion and providing a first fastener. The method also including the steps of securing the head portion of the bone plate to the condylar portion of the bone with the first fastener and providing a second fastener. The method also including the step of securing the body portion of the bone plate to the shaft portion of the bone by percutaneously securing the second fastener to the body portion of the plate and to the shaft portion of the bone while urging the shaft portion of the bone toward the condylar portion of the bone.

According to another aspect of the present invention, a locking plate system is provided to provide for percutaneous compression of the bone. The plating system includes a percutaneous target assembly for the plating system to target holes on a plate percutaneously. The assembly may be made of, for example a target arm, a handle, a connecting screw, two different style sheaths, and drill guides, a plug and a trocar. The plate itself has two different styles of holes which are locked, round holes, and oval compression holes. The target arm may reflect the same pattern as the plate. The target arm may be designed to target the center of both the round and the oval holes if compression is not needed or to provide compression by placing the screws eccentrically in the oval holes. The sheath, drill guide, and trocar assemblies may provide protection to the soft tissue during drilling. There may be two different shapes that may be designed to fit into the rounded oval holes. Since compression may not be desired all the time, the plug may be used to lock into the target arm and turn the oval hole into a round hole. The handle and the connecting screws may be used to attach the target arm to the plate and to hold it into position during the surgery.

The technical advantages of the present invention include the ability to interoperatively select between rigid and movable securement of the plates. For example, according to one aspect of the present invention, the fracture repair system of the present invention includes a bone plate that includes threaded holes and spaced apart clearance holes on the body of the plate. The threaded holes cooperate with fasteners having a threaded cap for rigid securement of the fastener to the plate. The fracture repair system further includes movable fasteners that include caps which are movably secured at the clearance holes on the body of the plate. Thus, the present invention provides for an interoperatively or in situ selection of rigid or movable securement of the plate.

Another technical advantage of the present invention is that the surgeon may interoperatively or in situ in the patient replace a fastener that has become stripped in the fracture repair system with a larger screw and maintain the rigid securement of the plate. For example, according to one aspect of the present invention, the fracture repair system includes a bone plate that has a threaded hole on the body of the bone plate. The fracture repair system further includes a first fastener which has threads on the cap portion of the fastener as well as small cortical threads on the stem portion of the fastener. If the bone mating with the cortical threads on the stem portion of the fastener becomes stripped, the first fastener may be removed from the bone plate and a second larger fastener, which has a threaded portion on the cap portion of the larger fastener with threads identical to that of the smaller fastener as well as cancellous larger threads on the stem portion of the second larger fastener. Thus, the present invention provides for interoperative use of a larger screw with rigid securement of the plate if the bone mating with the first installed smaller screw is stripped.

Yet another advantage of the present invention is that the bone fragments separated by trauma may be reconnected. For example, according to one aspect of the present invention, the fracture repair system of the present invention may include a plate having holes into which lag screws may be fitted. The fracture repair system further may include a lag screw or a screw having a portion of the stem void of threads. If the first bone fragment is connected to the head of the fastener and the second bone fragment is connected to the threaded portion of the lag screw as the lag screw is rotated, the bone fragments may be connected or drawn together. Thus, the present invention provides for the connection of separated bone fragments and resulting improvement of blood flow and healing.

The technical advantages of the present invention further include the ability to position a screw in a divergent direction diverging from the bone plate so that distal condyles and fragments thereof may be properly secured by the bone screw. For example, according one aspect of the present invention, a fracture repair system is provided which includes a bone plate with a cooperating bushing having a spherical periphery. The bushing therefore may be spherically rotated with respect to the bone plate and the bushing may receive a bone screw or fastener which may be fixedly secured at any position by tightening the bone screw to the plate utilizing the split bushing. Thus, the present invention provides for lockably securing a bone plate in diverging directions.

The technical advantages of the present invention further include the ability to pull large fragments together or in line with each other. For example, according to one aspect of the present invention, a fracture repair system is provided which includes a plate as well as a first attachment component associated with the plate and a second attachment component spaced from the first attachment component and associated with the plate such that the second attachment component when cooperating with the plate provides for compressive force having a component in the longitudinal axis of the plate to provide for compression of the bone. Thus, the present invention provides for pulling large fragments of the bone together in a line by compressing the bone.

The technical advantages of the present invention also include the ability to encourage a fast rate of healing of the fracture site. For example, according to one aspect of the present invention, a fracture repair system is provided which includes a plate as well as a first attachment component and a spaced apart second attachment component which second attachment component provides a compressive force to compress the bone at the fracture site. According to Wolff's law, load applied to a bone promotes bone growth. Thus, the present invention encourages a faster rate of healing of a fracture site by providing for a compression of the fracture site.

The technical advantage of the present invention further includes the ability to provide for a small scar when plating a bone fracture. For example, according to one aspect of the present invention, a fracture repair system is provided which includes a plate as well as a first attachment component and a second attachment component. The second attachment component is percutaneously inserted into the second feature of the plate. The percutaneously insertion of the second attachment component provides for a small scar. Thus, the present invention provides for a small scar associated with a plating of a bone fracture repair site.

The technical advantages of the present invention further include the ability to provide for reduced infection associated with the surgical plating of a bone fracture site. For example, according to one aspect of the present invention, a fracture repair system is provided which includes a bone plate as well as a first and second attachment component operatively associated with the bone plate. The second attachment component is percutaneously inserted into the bone plate. The percutaneous inserting of the second attachment component provides for a small incision and, therefore, a reduced area for infection and consequently, reduced infection. Thus, the present invention provides for reduced infection of the wound site.

The technical advantages of the present invention further include a shorter healing period associated with the percutaneous plating of a bone fracture site on a patient. For example, according to one aspect of the present invention, a fracture repair system is provided with a plate and a first and second attachment component which are associated with the plate. The second attachment component is percutaneously inserted into the plate. The percutaneous insertion of the second component provides for a much smaller insertion area and a smaller wound site. The smaller wound site provides for a shorter healing period. Thus, the present invention provides for a shorter healing period for a patient having a bone plating procedure related to a fracture of a bone.

The technical advantages of the present invention further include an ability to provide for percutaneously insertion of bone plate with a minimal amount of instrumentation. For example, according to one aspect of the present invention, a fracture repair system is provided which includes a guide which is modular. The modular components provides for an ability to mix and match components to provide for a variety of bone plate styles and lengths as well as to provide for a variation in the space of the guide from the plate to account for various size and weights of patients. Thus the present invention provides for a plating system which cooperates with a wide variety of plates with a minimal number of components.

Additional objects, features, and advantages of the invention will become apparent to those skilled in the art upon consideration of the following detailed description of the preferred embodiment exemplifying the best mode of carrying out the invention as presently perceived.

### BRIEF DESCRIPTION OF THE DRAWINGS

FIG. 1 is a plan lateral view of a fracture repair system in accordance with the present invention with a femur plate coupled to a femur and a tibia plate coupled to a tibia;

FIG. 2 is a perspective lateral view of a fracture repair system in accordance with the present invention with a femur plate coupled to a femur and a tibia plate coupled to a tibia;

FIG. 3 is a perspective view of a femur plate in accordance with the present invention;

FIG. 4 is a plan view of a the femur plate of FIG. 3;

FIG. 5 is an enlarged plan view of a the femur plate of FIG. 3;

FIG. 6 is a cross section view of the femur plate of FIG. 5 taken along lines 6-6;

FIG. 6A is a cross section view of the femur plate of FIG. 5 taken along lines 6A-6A;

FIG. 7 is a perspective view of a tibia plate in accordance with the present invention;

FIG. 8 is a plan view of a the tibia plate of FIG. 7;

FIG. 9 is a cross section view of the tibia plate of FIG. 8 taken along lines 9-9;

FIG. 10 is a plan view of a cannulated, cancellous bone screw for attachment to cancellous bone of a long bone for use with the fracture repair system of FIG. 1;

FIG. 11 is a partial cross sectional view of the tibia plate of FIG. 8 taken along lines 11-11 showing a portion of the tibia plate coupled to the tibia with the tibia plate in cross section and showing various bone screws positioned in the tibia;

FIG. 12 is a partial cross sectional view of the femur plate of FIG. 4 taken along lines 12-12 showing a portion of the femur plate coupled to the femur with the femur plate in cross section and showing two of the bone screws of FIG. 14 positioned in divergent positions in the condyles of the femur in accordance with the present invention;

FIG. 12A is a partial cross sectional view of the femur plate of FIG. 4 taken along lines 12A-12A showing a portion of the femur plate coupled to the femur with the femur plate in cross section and showing the bone screw of FIG. 10 positioned in the condyles of the femur;

FIG. 13 is a plan view of a cortical bone screw for attachment to both cortical bone surfaces of a long bone and for engagement with the bone plate for use with the femur plate of FIG. 4;

FIG. 14 is a plan view of a bone screw for engagement with the bone plate installed in a bone plate according to the present invention with a portion of the bone plate and a bushing providing polyaxial rotation shown in cross section;

FIG. 15 is a top view of a bushing for providing polyaxial rotation of the bone screw according to the present invention;

FIG. 16 is a plan view shown in cross section of the bushing of FIG. 15;

FIG. 17 is a plan view of a drill guide instrument installed on a bone plate for use with the fracture repair system of FIGS. 1-16;

FIG. 18 is a plan view of a cancellous bone screw for attachment to cancellous bone of a long bone for use with the fracture repair system of FIG. 1;

FIG. 19 is a plan lateral view of a fracture repair system in accordance with another embodiment of the present invention with a femur plate coupled to a femur and a tibia plate coupled to a tibia with the plates having fully locking and non-locking portions as well as fasteners including the locking fastener of FIG. 23 and the non-locking fastener of FIG. 13;

FIG. 20 is a perspective lateral view of the femur and tibia plate of the fracture repair system of FIG. 19;

FIG. 21 is a plan view of a the femur plate of FIG. 19, as well as, fasteners including the locking cortical screw of FIG. 23, the locking cancellous cortical screw of FIG. 24, the partially threaded lag screw of FIG. 25, the fully threaded cancellous screw of FIG. 18, the non-locking cortical screw of FIG. 13, the polyaxial locking cancellous screw of FIG. 14, the polyaxial locking cancellous screw of FIG. 26 and the cannulated cancellous screw of FIG. 12A;

FIG. 21A is a cross section view of the femur plate of FIG. 21 taken along lines 21A-21A in the direction of the arrows;

FIG. 21B is a cross section view of the femur plate of FIG. 21 taken along lines 21B-21B in the direction of the arrows;

FIG. 21C is a cross section view of the femur plate of FIG. 21 taken along lines 21C-24C in the direction of the arrows;

FIG. 22 is a plan view of the tibial plate of FIG. 19, as well as, fasteners including the locking cortical screw of FIG. 23, the non-locking cortical screw of FIG. 13, and the polyaxial locking cancellous screw of FIG. 14;

FIG. 22A is a cross section view of tibial femur plate of FIG. 22 taken along lines 22A-252 in the direction of the arrows; and

FIG. 23 is a plan view of a cortical bone screw for attachment to cortical bone of a long bone and with proximal locking threads for use with the fracture repair system of FIG. 19;

FIG. 24 is a plan view of a cancellous bone screw for attachment to cancellous bone of a long bone and with larger threads for use to replace the screw of FIG. 23 including proximal locking threads compatible with the proximal locking threads of the screw of FIG. 16 for use with the fracture repair system of FIG. 21;

FIG. 25 is a plan view of a partially threaded cancellous bone screw for attachment to cancellous bone of a long bone to connect bone fragments by lagging with the fracture repair system of FIG. 19;

FIG. 26 is a plan view of a bone screw smaller than the screw of FIG. 14 for engagement with the bone plate of FIG. 19 installed in a bone plate according to the present invention with a portion of the bone plate and a bushing providing polyaxial rotation shown in cross section;

FIG. 27 is a plan view of a K-wire for use with the bone plates of FIGS. 21 and 22;

FIG. 28 is a plan view of a fracture repair system in accordance with another embodiment of the present invention with a femur plate of FIG. 19. for coupling to a femur having fully locking portions and including the locking cortical screw of FIG. 23 and the larger locking cortical screw of FIG. 24;

FIG. 29 is a flow chart of a method of performing trauma surgery using the fracture repair system in accordance with another embodiment of the present invention;

FIG. 30 is a perspective view of a bone plate and guide of another embodiment of the present invention in the form of a fracture repair system for use with percutaneous compression of a bone fracture;

FIG. 31 is a plan view partially in cross section the fracture repair system of FIG. 30 showing the system in use with a screw to provide percutaneous compression;

FIG. 32 is a perspective view of a bone plate and guide of yet another embodiment of the present invention in the form of another fracture repair system for use with percutaneous compression of a bone fracture;

FIG. 33 is a plan view partially in cross section of bone plate of the fracture repair system of FIG. 32 showing a bushing of the guide in alignment with a hole of the bone plate;

FIG. 34 is a perspective view of a sheath and drill guide assembly for use with the guide of the fracture repair system of FIG. 32;

FIG. 35 is a plan view of the sheath and drill guide assembly of FIG. 34;

FIG. 36 is a perspective view of the bone plate and guide of FIG. 32 showing the sheath and drill guide assembly of FIG. 35 in position on the guide with a drill in position to be inserted into the drill guide;

FIG. 37 is a perspective view of the bone plate and guide of FIG. 32 showing the sheath of the sheath and drill guide assembly of FIG. 35 in position on the guide with an anchor bolt in position to be inserted into the drill guide;

FIG. 38 is a perspective view of the bone plate and guide of FIG. 32 showing the sheath of the sheath and drill guide assembly of FIG. 35 in position on the guide with an anchor bolt and an anchor clip in position in the drill guide;

FIG. 38A is a plan view partially in cross section of the targeting guide of the fracture repair system of FIG. 38 showing the anchor bolt in a first and a second position in a hole of the targeting guide;

FIG. 38B is plan view partially in cross section of the bone plate of the fracture repair system of FIG. 38 showing the threaded portion of anchor bolt in a hole of the bone plate;

FIG. 38C is a top view partially in cross section of the targeting guide of the fracture repair system of FIG. 38 showing a the anchor clip in use urging the anchor bolt against a side of the hole of the targeting guide;

FIG. 39 is an exploded perspective view of the fracture repair system of FIG. 38;

FIG. 40 is a perspective view of an oval sheath for use with the guide of the fracture repair system of FIG. 38;

FIG. 41 is a plan view of the oval sheath of FIG. 40;

FIG. 42 is a perspective view of fracture repair system of FIG. 38 showing the oval sheath of FIGS. 40 and 41 and the compression drill bushing in position on the guide;

FIG. 43 is a plan view partially in cross section of FIG. 42;

FIG. 44 is a perspective view of a compression drill bushing for use in the oval sheath for use with the guide of the fracture repair system of FIG. 38;

FIG. 45 is a plan view of the compression drill bushing of FIG. 44;

FIG. 46 is a perspective view of the fracture repair system of FIG. 38 showing the compression drill bushing of FIG. 44 in position in the oval sheath and a drill in position adjacent the drill bushing;

FIG. 47 is a partial plan view partially in cross section of FIG. 46 showing the drill bushing in position over the plate oval opening;

FIG. 48 is a perspective view of the fracture repair system of FIG. 38 showing a fastener and a fastener driver in position over the oval sheath;

FIG. 49 is a partial plan view partially in cross section of FIG. 48 showing the fastener in position over the plate oval opening;

FIG. 50 is a partial plan view partially in cross section of FIG. 48 showing the fastener in position over the plate oval opening after a subsequent compression has occurred;

FIG. 51 is a perspective view of fracture repair system of FIG. 38 showing the oval sheath of FIGS. 40 and 41 in a second position on the guide for performing a subsequent or second compression of the bone;

FIG. 52 is a perspective view of the fracture repair system of FIG. 38 showing the compression drill bushing of FIG. 44 in the position of the oval sheath of FIG. 51 and a drill in position adjacent the drill bushing;

FIG. 53 is a partial plan view partially in cross section of FIG. 52 showing the drill in position over the plate;

FIG. 54 is a perspective view of the fracture repair system of FIG. 38 showing a fastener and a fastener driver in position over the oval sheath position of FIG. 51;

FIG. 54A is a partial plan view partially in cross section of FIG. 52 showing the threaded portion of the anchor bolt in a first and a second position in a threaded hole of the plate;

FIG. 54B is a partial plan view of another embodiment of a plating system of the present invention with a larger theaded hole showing the threaded portion of the anchor bolt in a first, a second and a third position in a threaded hole of the plate;

FIG. 55 is a partial plan view partially in cross section of FIG. 54 showing the fastener in position over the plate;

FIG. 56 is a perspective view of the fracture repair system of FIG. 38 showing a non-compression orienting plug and the round sheath of the assembly of FIG. 34 in position in the guide of the repair system of FIG. 51;

FIG. 57 is a partial cross sectional view of the targeting guide of FIG. 56 showing the plug in position in a hole of the targeting guide;

FIG. 58 is a partial plan view partially in cross section of the bone plate of FIG. 54 showing the non-compression orienting plug in position over a cylindrical opening of the plate;

FIG. 58A is a partial plan view partially in cross section of the bone plate of FIG. 54 showing the non-compression orienting plug in position over a threaded opening of the plate;

FIG. 59 is a perspective view of the non-compression drill bushing for use in the oval sheath for use with the guide of the fracture repair system of FIG. 38;

FIG. 60 is a plan view of the non-compression drill bushing of FIG. 59;

FIG. 61 is a perspective view of the fracture repair system of FIG. 38 showing the non-compression drill bushing of FIG. 59 in position in the guide of the repair system of FIG. 38;

FIG. 62 is a partial plan view partially in cross section the fracture repair system of FIG. 61 showing the drill bushing in position over the plate;

FIG. 63 is a plan view of a the fracture repair system according to the present invention for use in repairing tibia, femoral, humeral and ulna fractures; and

FIG. 64 is a flow chart of another method of performing trauma surgery using the fracture repair system in accordance with another embodiment of the present invention.

### DETAILED DESCRIPTION

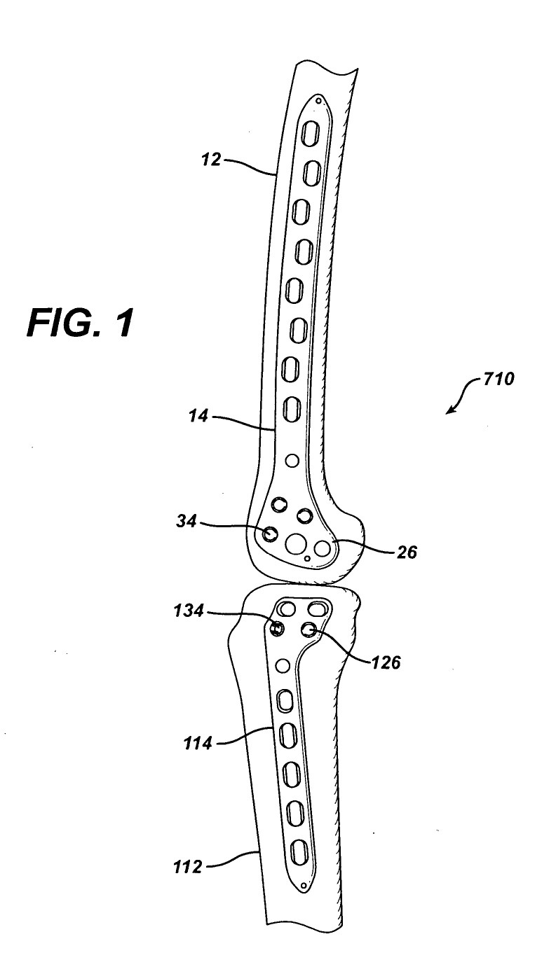

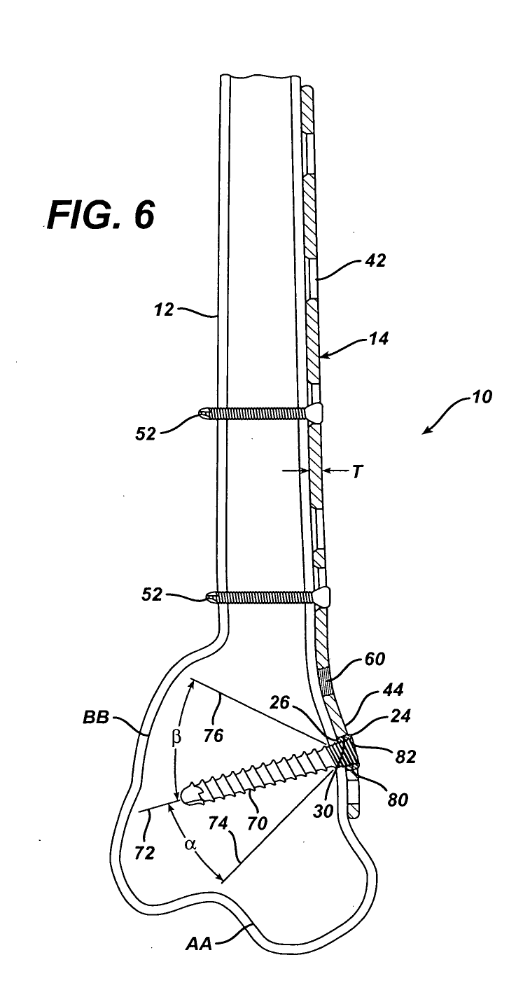

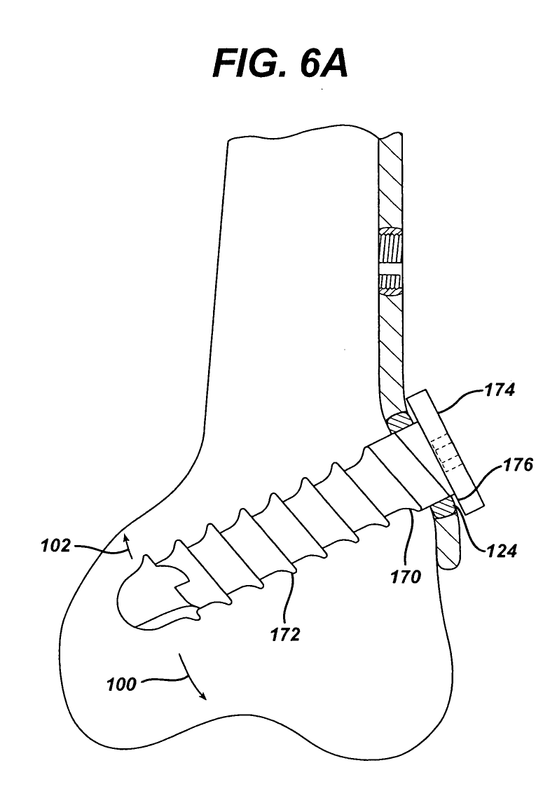

According to the present invention and referring now to FIG. 6, a fracture repair system 10 is shown for engagement with a long bone 12. The long bone 12 may be any long bone, for example, a femur, tibia, fibula, humerus, radius or ulna, but as shown in FIG. 1 the long bone is a femur. The fracture repair system 10 includes a plate 14.

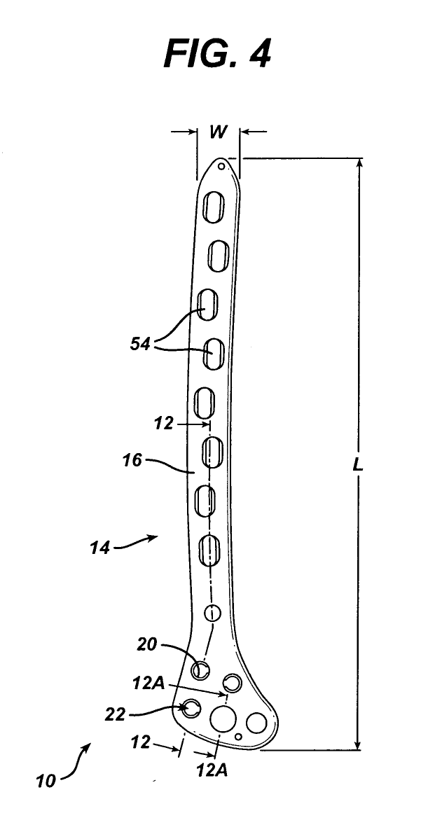

Referring now to FIG. 4 the fracture repair system 14 is shown in greater detail. The plate 14 may be made of any suitable durable material and may, for example, be made of a metal, for example, a metal compatible with the human anatomy, for example, cobalt chrome, stainless steel or titanium. The plate 14 includes a body portion 16 and an interior wall 20. The interior wall 20 defines a plate hole 22 through the body portion 16.

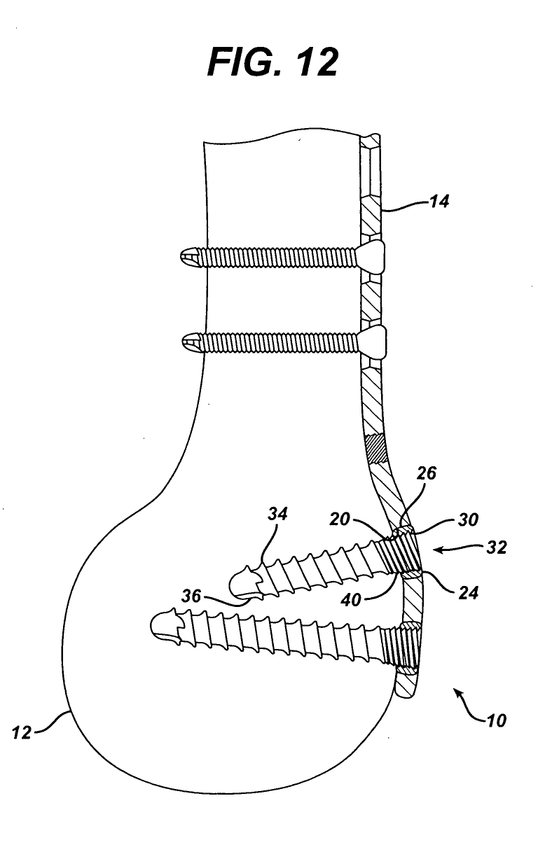

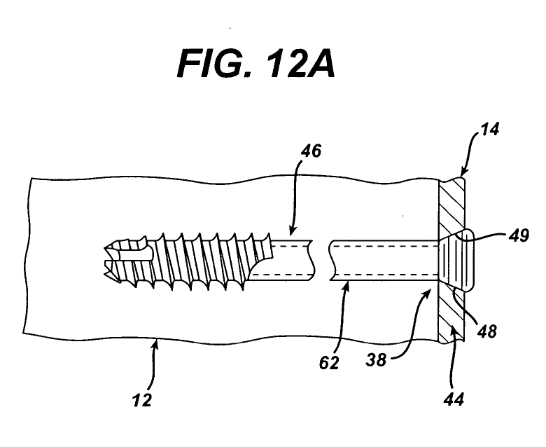

Referring now to FIG. 12 the fracture repair system 10 is shown in greater detail. In addition to the plate 14 the fracture repair system 10 includes one or more bushings 24. The bushing 24 includes a radial exterior surface 26 and opposite radial interior surface 30. The opposite radial interior surface 30 defines a passageway 32 through the bushing 24. The exterior surface 26 of the bushing 24 and the interior wall 20 of the plate 14 are configured to permit the polyaxial rotation of the bushing 24 within the plate hole 22. (see FIG. 12) Such polyaxial rotation may be permitted by providing an arcuate or spherical surface on the interior wall 20 of the plate 14 and a mating arcuate or spherical surface on the radial exterior surface 26 of the bushing 24.

The fracture repair system 10 further includes the attachment component 34 includes a distal portion 36 sized for current passage through the passageway 32 and into the long bone 12. The attachment component 34 further includes an opposite proximal portion 40 sized to press the bushing 24 against the internal wall 20 of the plate 14 to form a friction lock between the bushing 24 and the plate 14 in a selected polyaxial position. The attachment component 34 is positionable in an orientation extending divergently from the center of the plate.

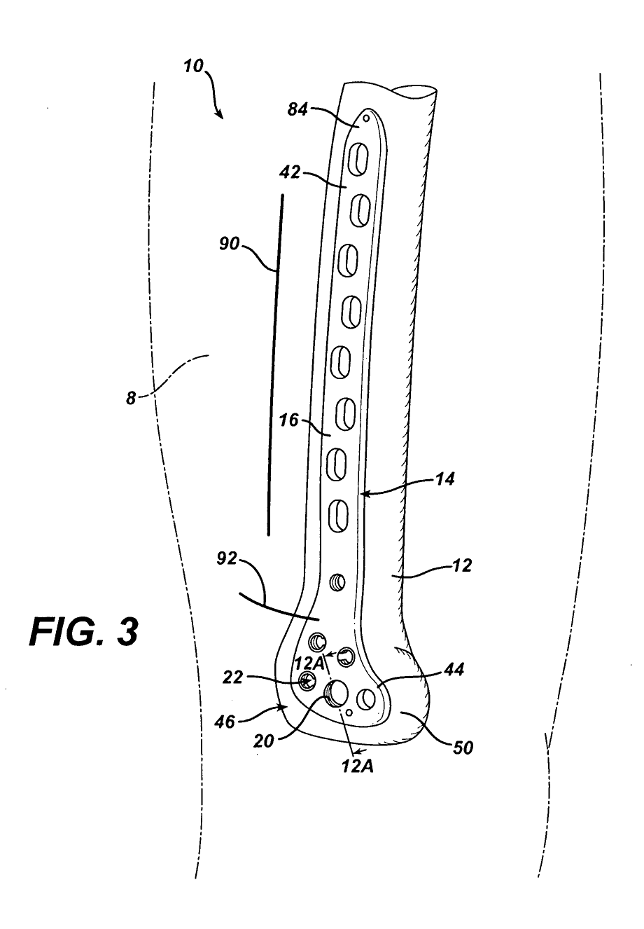

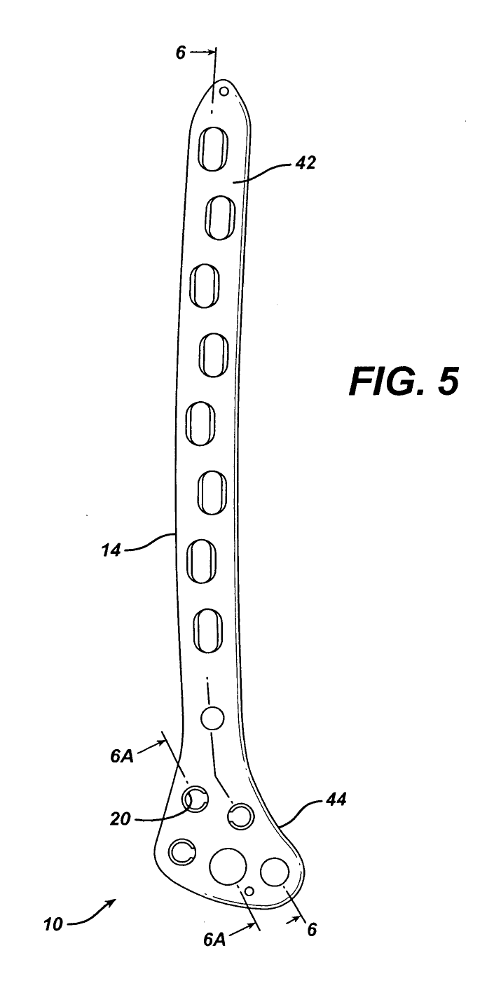

Referring now to FIGS. 3 through 6 the fracture repair system 10 is shown as femur plate assembly 10. Preferably and as shown in FIGS. 3-6, the body portion 16 of the femur plate 14 preferably includes a proximal portion 42 and distal portion 44. To provide for optimal support of the femur, the femur plate 14 has a shape generally conforming to the outer periphery of the femur 12. The proximal portion 42 of the bone plate 14 is generally flat or planer in conforming to the general flat or planer nature of the proximal shaft portion of the femur. The femur plate 14 may have a bow to accommodate the natural anterior/posterior bow of the femur 12. The distal portion 44 of the femur plate 14 has a shape generally conforming to the condylar portion 46 of the femur 12. Since the condylar portion 46 of the femur 12 is arcuate or curved the distal portion 44 of the femur plate 14 is preferably curved to mate with condyles 50 of the condyle portion 46 of the femur 12.

While the particular size and shape and dimensions of the femur plate 14 may vary widely depending upon the size of the femur on which it is installed, for an adult human femur, the plate 14 may have, for example as shown in FIG. 4, an overall length L of about 6 to 14 inches and a width W of about 1¼ to 3/4 of an inch and a thickness T of, for example as shown in FIG. 6, about ⅛-¼ inch. Since the human anatomy is generally symmetrical, the femur plate 14 is either a right hand or left hand femur plate and the right hand and left-hand femur plates are different, but generally symmetrical with each other.

While the fracture repair system of the present invention includes one or more bushings which cooperates with an attachment component such that the attachment component may be positionable in an orientation diverging from the center of the plate, it should be appreciated that the fracture system or plate may include a plurality of attachment components. Further these attachment components may be of different styles or types.

Referring now to FIG. 6, the femur plate 14 is shown with three different types of attachment components. Solid, fully-threaded, cortical screws 52 are positioned in elongated openings 54 shown in FIG. 4 in the proximal portion 42 of the femur plate 14. The fully-threaded cortical screws 52 may, as shown in FIG. 6, be self-tapping and cut threads while they are being screwed through the plate into the bone during surgery. The cortical screws 52 are supported primarily by the cortical bone to which they have been secured. While the proximal portion 42 of the bone plate 14 may be secured by a solitary cortical screw 52 preferably and, as shown in FIG. 6, the proximal portion 42 of the femur plate 14 is supported by a series of several spaced apart fully threaded cortical bone screws.

To provide ample support for the proximal portion 42 of the plate 14 and to provide for a standard commercially available femur plate 14, the femur plate 14 preferably includes a uniformly spaced apart pattern of elongated openings 54 shown in FIG. 4. The surgeon may choose any of a number of the elongated openings 54 shown in FIG. 4 in which to drill and screw the cortical screws. Depending on the position of the fractures as few as two or three cortical screws may be sufficient to support the femur plate 14.

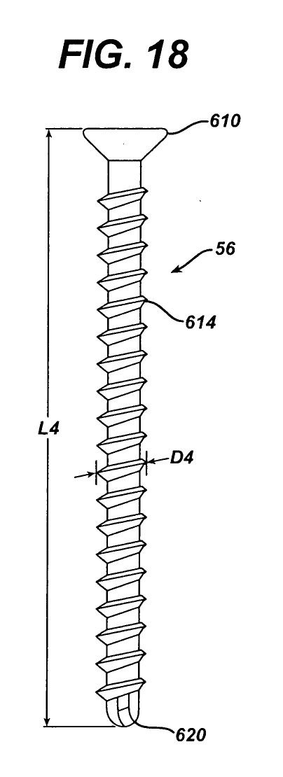

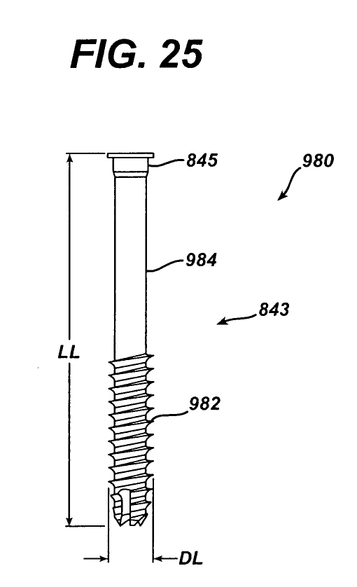

Continuing to refer to FIG. 6, cancellous screws 56 (see FIG. 18) or screw 980 (see FIG. 25) may also be placed in the elongated openings 54 and used to secure the proximal portion 42 of the femur plate 14.

The screw 56 or 980 unlike cancellous screw 70 (see FIG. 6) does not include threads on the head of screw 56 or 980. The lack of screw threads on the head of screw 56 or 980 allows the head to spin on the bushing 24 without locking, thereby achieving a lagging effect. The cancellous screws 56 or 980 may be any suitable size and may, for example, be 2 to 6 millimeters solid, cancellous, partially or fully threaded cancellous screws. The cancellous screws 56 or 980, preferably have a length less than the thickness of the femur so that they may not protrude from the opposite surface of the femur.

Distal portion 44 of the femur plate 14 is designed to follow the general contours of the lateral distal femur while the proximal portion 42 incorporates the natural bow of the femur.

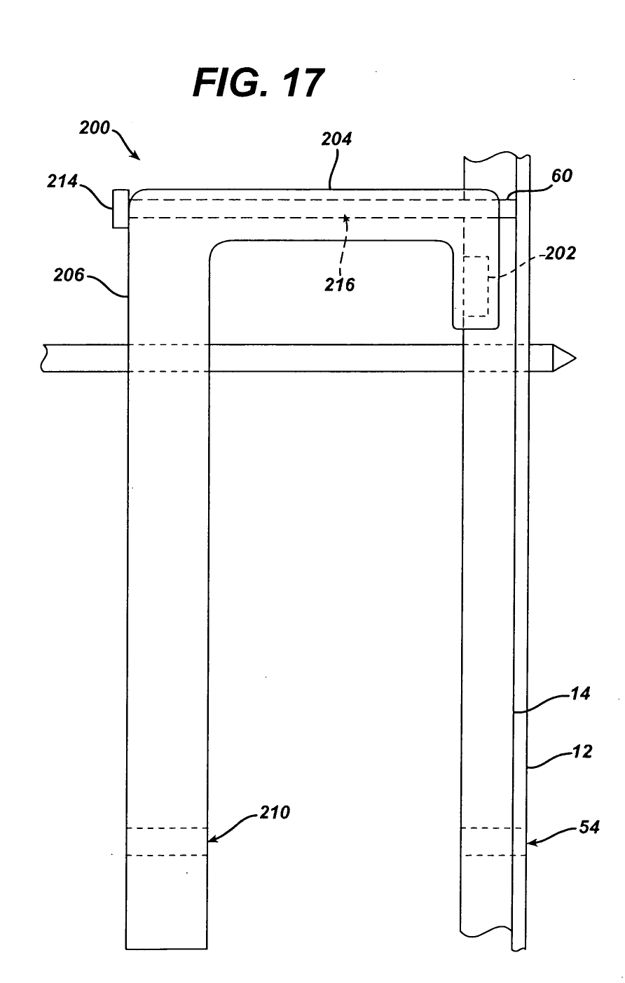

The femur plate 14 may include one or more tapped openings 60 in the femur plate 14 which may be utilized to secure a drill guide 200 shown in FIG. 17 for aligning a drill and a screwdriver for the insertion of the screws 52 and 56 or 980 into the femur. The drill guide 200 will be described in greater detail later.

According to the present invention, the plate 14 includes attachment components which are positionable in an orientation diverging from the center of the plate. The plate 14 thus includes at least one screw 70 which is secured to the plate 14 by means of the bushing 24. The screw 70 may be in the form of a cancellous screw. The cancellous screw is particularly well suited for securing the condylar portion of the distal portion of the femur. The cancellous screw 70 may be partially or fully threaded and may have any suitable length to reach the proper portion of the fractured condylar portion of the distal femur. For example, the cancellous screw may have a length from 20 to 150 millimeters. The cancellous screw may have a suitable diameter to properly secure the fractured portions of the femur. For example, the cancellous screw may have a diameter of 3 to 10 millimeters. The cancellous screw 70 is used to secure the distal portion of the femur plate to the bone.

The cancellous screws may be rotated from the first position 72 shown in solid to position 74 shown rotated an angle α or to a third position 76 rotated in the opposite direction an angle β (see FIG. 6). If the cancellous screw 70 is rotated to the second position 74, the screw 70 will be utilized to secure fragment AA while, if the cancellous screw 70 is rotated to position 76, the cancellous screw 70 may be utilized to secure fragment BB. The tip of the cancellous screws 70 can therefore be rotated in a conical pattern.

The cancellous screw 70 as shown in FIG. 6 may include external threads 80 on the head or proximal portion of the screw 70. Alternatively, the head or proximal portion may have a smooth conical head. The external threads 80 mate with internal threads 82 on the bushing 24. Preferably and as shown in FIG. 6 the external threads 80 are tapered such that as the external threads 80 of the screw 70 are engaged into the bushing 24 the bushing 24 expands, locking the radial external surface 26 of the bushing 24 to the radial interior surface 30 of the plate 14.

By permitting the bushing 24 to rotate within the plate 14 and by permitting the bushing 24, the screw 70 and the plate 14 to all be locked securely in place, the screw may be fixedly positioned in many different orientations, while maintaining all components at minimal stress. As shown in FIG. 6, the feature of having the positionable screw and plate configuration permits either fragment AA or fragment BB to be secured by the screw 70.

Referring now to FIG. 6A, one or any portion of the locations of the plate 14 may include one or more bushings 124 which may alternatively be utilized with a screw having a non-threaded head. For example, as shown in FIG. 6A a cancellous screw 170 is shown similar to screw 70 but not including external threads on the head of the screw. The screw 170 does include cancellous threads 172 for securement to the cancellous bone. The screw 170 includes a head 174 which is secured against face 176 of bushing 124. In this configuration the bushing 124 serves to permit the rotation of the screw 170 in the direction of arrows 100 and 102, thus permitting the orientation of the screw 170. The use of the bushing 124 prevents stress risers on the head 174 or face 176 of the bushing 124.

The plate 14 may be made of any suitable durable material that is biologically compatible with the human anatomy and preferable made of a high strength metal. For example, the plate may be made of stainless steel, cobalt chrome or titanium. Preferably the plate 14 is manufactured from a forged or wrought titanium alloy. One such suitable alloy is ASTM F-620-97 and another suitable alloy is ASTM F-136 ELI.

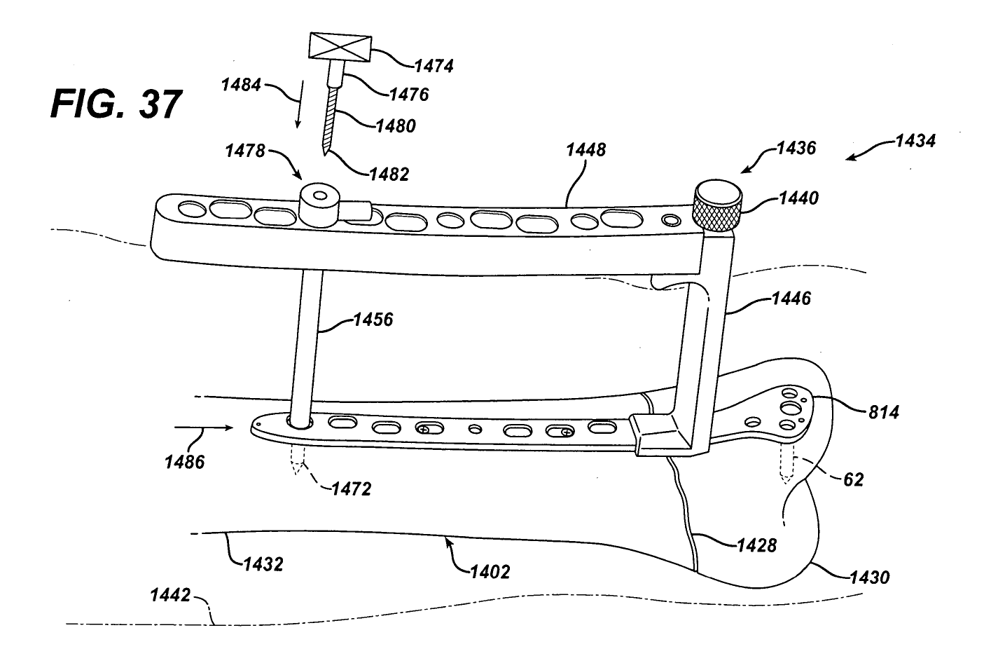

Referring to FIG. 37, the femoral plate 14 may be secured to the femur 12 during surgery either percutaneously or by conventional open surgery. When the femur plate 14 and screws are implanted in conventional open surgery a longitudinal cut 90 is made through the skin along the thigh 8 laterally where the femur plate is typically installed. A lateral installation of the femur plate provides for the minimal interference with muscle, ligaments and other soft tissue. The longitudinal cut 90 in the thigh 8 through the skin parallel to the femur 12 is made approximately the length of the femur plate 14 and the soft tissue is pulled apart so that the femur plate may be placed in position. Cancellous and cortical screws are then positioned over their respective openings in the femur plate 14 and secured to the femur 12.

When performing percutaneous surgery the skin of the thigh 8 is opened laterally near the knee and a transverse cut 92 is made and femur plate 14 is inserted at that opening and guided against the femur 12 proximally toward the hip. The proximal end of the femur plate 14 may include a contoured tip 84 to ease the percutaneous installation of the femur plate 14.

While the femur plate 14 may be made of any suitable size depending on the size of the human in which the plate is to be installed, the femoral plates 14 may be available in various lengths so that they will be available when trauma strikes. For example, the femoral plates may be provided with varying lengths including for example 5, 8, 11, 14 or 18 screw holes in the shaft.

The cortical and cancellous screws are manufactured of any suitable durable material that are typically manufactured of a wrought titanium alloy for example ASTM F-136 ELI.

Referring now to FIG. 12A, the femur plate 14 may further include a cannulated cancellous screw for positioning in the condylar portion 46 of the femur 12. The cannulated cancellous screw 62 preferably has a length slightly shorter than the length of the portion of the cancellous bone at the condylar portion 46 such that the cannulated cancellous screw does not contact the opposite cortical bone. The cannulated cancellous screw may, for example, be 8 millimeter cannulated cancellous and preferably as shown in FIG. 12A include external threads 48 located on proximal portion 38 of the cancellous screw 46. The external threads 48 mate with the internal threaded opening 49 of the distal portion 44 of the femur plate 14. The cannulated cancellous screw 62 provides additional structural support to the condylar portion 46 of the femur 12. Alternatively, the head of the cancellous screw 62 may be smooth, thereby allowing the head to spin in the plate without locking. The spinning achieves a lagging effect, i.e. drawing the fragments together.

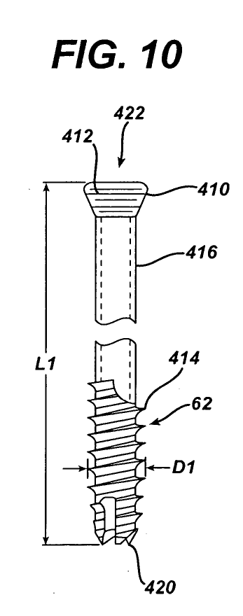

Referring now to FIG. 10, the cannulated cancellous screw 62 is shown in greater detail. The cannulated cancellous screw may not be in any particular size and may include a diameter D1 of, for example, 4 to 10 millimeters. The cannulated cancellous screw has a length L1 sufficient to occupy most of the condylar portion 46 of the femur or long bone 12. To provide for rigid attachment of the cannulated cancellous screw 62 to the bone plate 14, the cancellous screw 62 preferably includes a head 410 having external threads 412 which may mate with internal threaded opening 49 of the bone plate 14 (see FIG. 12A). The cannulated cancellous screw 62 includes external threads 414 and may include an unthreaded shank portion 416. The cannulated cancellous screw 62 may include a self-tapping tip 420 which may also serve as a self-drilling as well as a self-tapping tip. As shown in FIG. 10, the threads 412 on the head 410 are tapered to provide for a tight locking fit with the bone plate 14. The cannulated cancellous screw 62 is by definition cannulated or includes a central longitudinal opening 422.

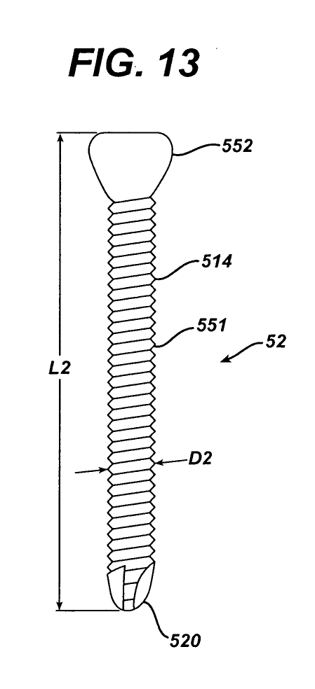

Referring now to FIG. 13 a cortical screw 52 is shown. The cortical screw 52 includes threads 514 which are adapted for securing cortical bone. The cortical screw 52 may include an unthreaded shank portion (not shown). The cortical screw 52 includes a head 552 which may, as shown in FIG. 13, have a generally oval shape. The cortical screw 52 may also include a self-tapping tip 520 which may also include self-drilling provisions. The cortical screw 52 has a length L2 which preferably is of sufficient length to engage the cortical bone on the opposite or exit side of the bone. The cortical screw 52 further includes a thread diameter D2 which is of sufficient size to provide sufficient holding power and engagement with the cortical bone. For example and as shown in FIG. 13, the cortical screw 52 has a diameter D2 of, for example, 3.5 to 6 millimeters.

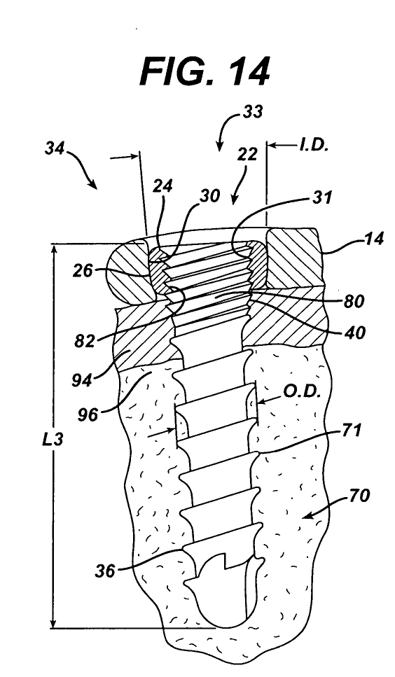

Referring now to FIG. 14 an attachment component according to the present invention is showed as cancellous screw 70. The screw 70 includes a distal portion 36 which has an outside diameter OD which is less than the inside diameter ID of the internal wall or surface 30 of the plate hole 22. The screw 70 further includes external threads 80 located on the proximal portion 40 of the screw 70. Preferably and as shown in FIG. 14, the external threads 80 are tapered. The external threads 80 are mateably engageable with the internal threads 82 on the bushing 24. The bushing 24 is pivotally engageable with the plate 14. The radially exterior surface 26 of the bushing 24 has a generally spherical shape and is mateably fitted with the interior wall or surface 30 of the plate hole 22. The interior threads 82 of the bushing 24 is larger than the outside diameter of cancellous threads 71 on the screw 70 to permit the distal portion of the 36 of the screw 70 to slidably pass or thread through the plate hole 22. The cancellous threads 71 are adapted for efficient engagement with cancellous bone 96 and the screw 70 has a length L3 which is sized to provide for the cancellous thread 71 to engage a significant portion of the cancellous bone 96.

Referring now to FIG. 18, a fully threaded cancellous screw 56 is shown for use with the bone plate 14. The cancellous screw 56 includes a head 610. The head 610 may have any suitable shape and may, for example, be flat head as shown in FIG. 18 or have a pan head shape. The screw 56 unlike cancellous screw 70 (see FIG. 6) does not include threads on the head of screw 56. The lack of screw threads on the head of screw 56 allows the head to spin on the bushing 24 without locking, thereby achieving a lagging effect. The cancellous screw 56 has a length L4 to provide for engagement with a suitable portion of the cancellous bone (not shown). The cancellous screw has threads 614 which are adapted for engagement with cancellous bone. The thread 614 has a diameter D4 which is sized for efficient and effective support and engagement with the cancellous bone. For example, the cancellous screw 56 may have a diameter D4 of, for example 2 to 6 millimeters. The cancellous screw 56 further includes a tip 620. The tip 620 may optionally include self-drilling and/or self-tapping features.

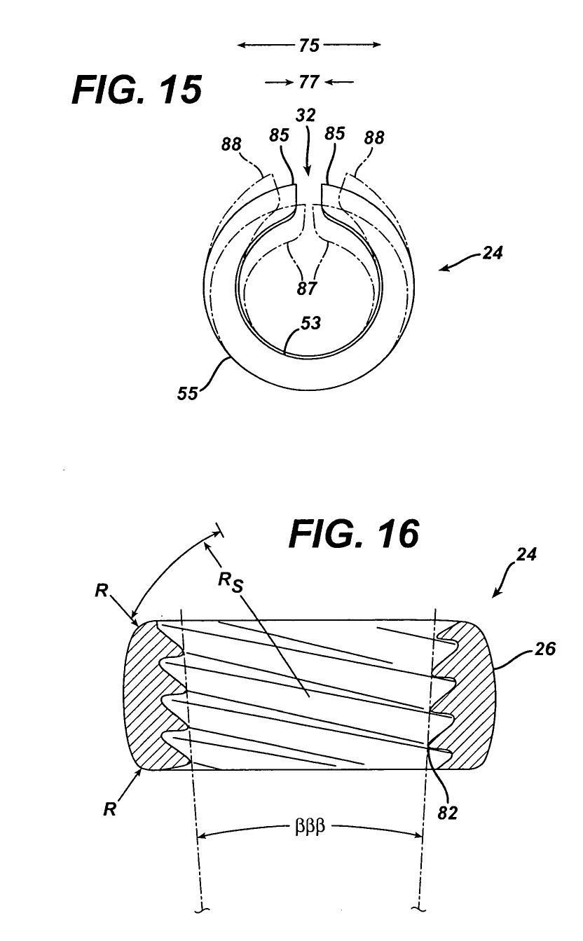

Referring now to FIG. 15, the bushing 24 is shown in greater detail. The bushing or collet is manufactured of any suitable durable material that is compatible with the human body. For example the collet may be made of cobalt chrome, stainless steel or titanium. For example the bushing 24 may be manufactured of a wrought titanium alloy. Such a wrought titanium alloy is ASTM F-136 ELI.

The bushing 24 preferably includes a radial opening or passageway 32 on the periphery of the bushing 24. The passageway 32 extends from the radially exterior surface 55 through the opposite radially interior surface 53. The bushing 24 has a first relaxed position 85 which represents the shape of the bushing 24 when not assembled into the plate 14. The bushing 24 further has an assembled position 87 as shown in the dotted line. The assembled position 87 represents when the bushing 24 is placed within the plate 14 and when the screws are not installed. The bushing 24 further has an expanded position 88 shown in phantom in which the bushing 24 is shown with the bushing 24 installed in the plate 24 and the screws installed within the bushing 24.

As can be seen in FIG. 15, the bushing 24 is contracted when the assembled position 87 to provide for an interference fit between the bushing 24 and the plate 14. Further as shown in FIG. 15, the bushing 24 is expanded as it moves from the assembled position 87 to the expanded position 88. This occurs because the tapered threads during engagement cause the bushing 24 to enlarge. The enlarging of the bushing 24 causes a tighter interference between the bushing 24 and the plate thereby securely locking the bushing in its polyaxial oriented position with minimal stress.

Referring now to FIG. 16 a cross-section of the bushing 24 is shown. As shown in FIG. 16 preferably the bushing 24 has a spherical radius R_{S} which defines the radial exterior surface 26 of the bushing 24. By providing a spherical radius R_{S} the bushing 24 may be oriented into a number of angular positions with respect to the plate.

Referring to FIG. 16 the internal threads 82 of the bushing 24 have a taper defined by an internal angle βββ. The angle βββ may be, for example, from 3 to 30 degrees. As shown in FIG. 16 the truncated spherical shape of the radial exterior surface 26 may be modified by corner radius R.

While the fracture repair system of the present invention includes the bushing to provide for positioning of the attachment component in a variety of diverging directions while providing for reduced stress at the plate, when percutaneously securing a bone screw to a bone plate location which does not provide for the pivotal securement of the bushing arrangement, it is critical that the screws in such fixed locations be properly positioned.

Referring now to FIG. 17 preferably the femur plate 14 is used in conjunction with drill guide 200. Drill guide 200 is installed onto the femur plate 14 during surgery and is utilized to guide drills and screwdrivers to properly orientate the screws that are placed in the proximal portion of the plate 14. The drill guide 200 includes a locating feature 202 in the form of, for example, an elongated pin which closely fits to the elongated slots of a plate. The drill guide includes a riser portion 204 and a bar portion 206 which is positioned parallel and spaced from the plate 14.

The bar portion 206 includes a series of bushing holes 210 which are in alignment with the center of the elongated openings 54 in the plate 14. To properly secure the drill guide 200 to plate 14, for example, the drill guide 200 may include a securing screw 214 which may be slidingly fitted to an opening 216 in the riser portion 204 and which may be secured to tapped opening 60 in the plate 14.

The drill guide 200 may be utilized both in conventional open surgery and in percutaneous surgery. When utilized in percutaneous surgery the bushing holes 210 may be utilized to guide trocars which will open the skin and tissue around the openings permitting the screws to be properly secured. Since the human anatomy is generally symmetrical, the drill guide 200 is either a right hand or left hand drill guide and the right hand and left-hand drill guide are different, but generally symmetrical with each other. It should be appreciated that the drill guide may be utilized for any bone plate for supporting any long bone for example a tibia, humerus, ulna, radius or fibula.

While heretofore the fracture repair system has been described in more detail as a femur plate, it should be appreciated that the plate may be utilized for supporting any long bone for example a tibia, humerus, ulna, radius or fibula.

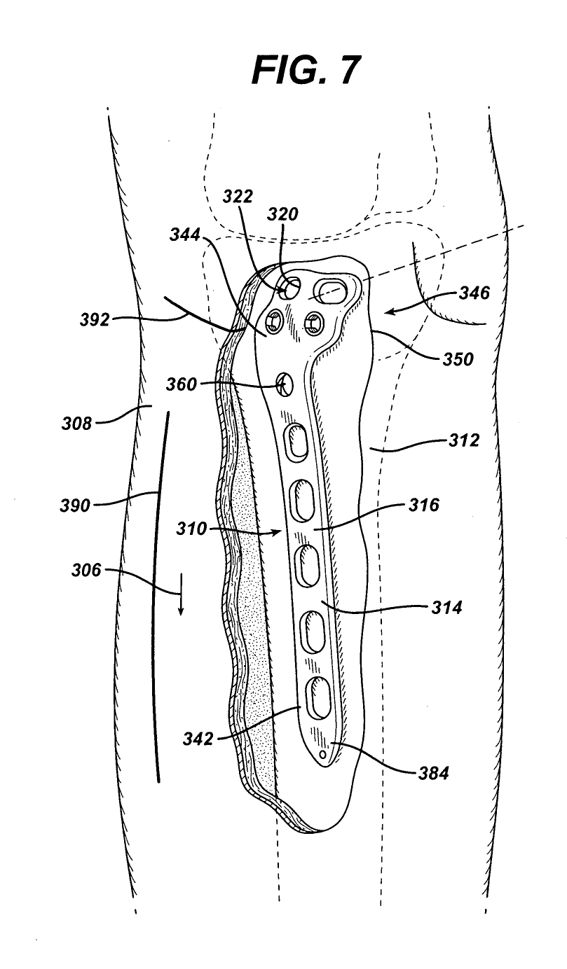

Referring now to FIG. 7-9 and 11, a tibia plate 314 for installation onto a tibia 312 is shown. The fracture repair system 310 for use on the tibia 312 includes a tibia plate 314 having a body portion 316. The body portion 316 includes a distal portion 342 and a proximal portion 344. The tibia plate 314 like the femur plate 14 is preferably positioned laterally on the long bone. The lateral position of the tibia plate reduces the amount of soft tissue that must be dislocated to position the tibia plate 314. Since the human anatomy is generally symmetrical, the tibia plate 314 is either a right hand or left hand tibia plate and the right hand and left-hand tibia plates are different, but generally symmetrical with each other. The proximal portion 344 of the tibia plate 314 is designed to follow the general contours of the lateral proximal tibia. The proximal portion 344 of the plate 314 is contoured to fit the lateral condyle 350 of the condylar portion 346 of the tibia 312. The body portion 316 of the tibia plate 314 like the body portion 16 of the femur plate 14 has a generally arcuate cross-section to conform to the distal shaft of the tibia 312.

The tibia plate 314 like the femur plate 14 may be made of any suitable durable material that is compatible with the human immune system and may for example be made of a durable non-corrosive material such as stainless steel, cobalt chrome or titanium. For example, the tibia plate may be manufactured from a forged or wrought titanium alloy. For example, such a titanium alloy may be ASTM F-620-97 or ASTM F-136 ELI.

Referring now to FIG. 7, the tibia plate 314 may be inserted into the human anatomy percutaneously or by conventional open surgery. When inserted by conventional open surgery, the leg 308 is cut with a longitudinal incision 390 of length roughly equal to that of the tibia plate 314. The soft tissue is moved away from the tibia 312 and the tibia plate 314 is placed against the tibia 312. Screws such as those for the femur plate are utilized to secure the tibia plate 314 to the tibia 312. If the tibia plate 314 is to be inserted percutaneously, a smaller longitudinal incision 392 is made in the skin of the leg 308 near the knee and the distal portion 342 of the body portion 316 of the tibia plate 314 is inserted in the incision 392 in the direction of arrow 306 toward the distal portion of the leg. A contoured tip 384 on the distal portion of the 342 of the tibia plate of the tibia plate 314 is shaped to ease the insertion of the tibia plate along the contour of the tibia 312 in the direction of arrow 306.

For installation either percutaneously or by conventional open surgery of the tibia plate 314 drill guides (not shown) such as drill guide 200 for the femur plate as shown in FIG. 17 are utilized. Again, as with the femur plate, the drill guide may be utilized to guide the drill and the screws whether the plate and screws are inserted percutaneously or by conventional open surgery. It should be appreciated that a left-hand drill guide (not shown) and a right hand drill guide (not shown) are necessary respectively for the right hand and left-hand tibia plates (not shown).

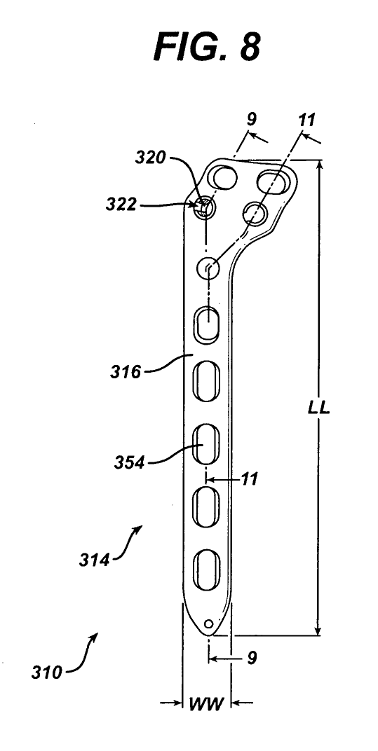

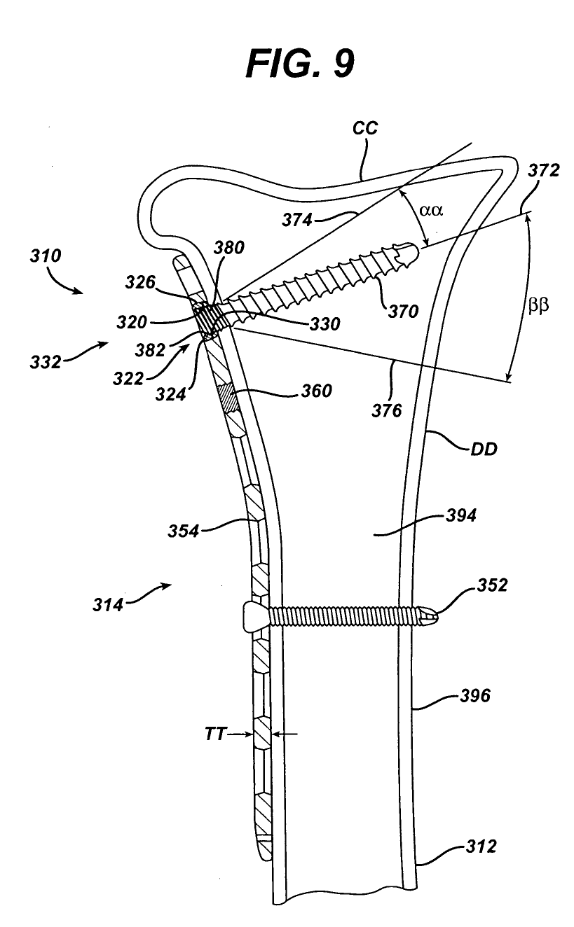

Referring now to FIG. 8, the tibial plate 314 may be made of sufficient dimensions to properly support the tibia 312. The proper dimensions of the tibial plate 314 are dependent thus on the size of the particular tibia to be treated as well as the inherent strength of the material from which the tibial plate 314 is made. For example, the tibial plate 314, if made of titanium, may have a thickness TT (see FIG. 9) of, for example, approximately 1/16 to 1/14 of an inch and a WW width of around ¼ to ¾ inch and a length LL of, for example, from 5-10 inches. To provide for a range of standard tibial plates, the tibial plates may be provided in varying lengths of, for example, a length with a number of elongated openings 354 of, for example, 4, 7, 11, or 14 elongated openings.

According to the present invention and referring to FIGS. 7-9 and 11, the tibial plate 314 includes the body portion 316 which conforms at least partially to the contour of the tibia 312. The tibia plate 314 also includes an interior wall 320 which defines a tibia plate hole 322 through the body portion 316.

Referring to FIG. 9, the tibial plate 314 further includes one or more bushings 324. The bushing 324 includes a radially exterior surface 326 and an opposite radially interior surface 330. The opposite radially interior surface 330 defines a passageway 332 there through. The exterior surface 326 of the bushing 324 and the interior wall 320 (see FIG. 9) of the plate 314 cooperate with each other and are configured to permit polyaxial rotation of the bushing 324 within the plate hole 322. The tibial plate 314 further includes an attachment component 370 in the form of, for example, a cancellous screw. The screw 370 includes a distal portion 336 sized for clearance passage through the passageway 332 and into the cancellous bone 394.

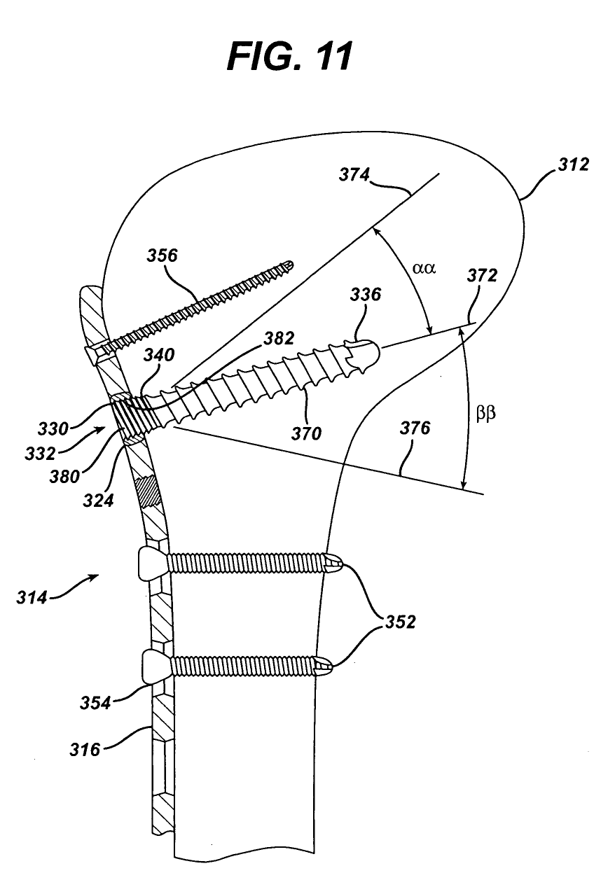

The screw 370 further includes a proximate portion 340 sized to press the bushing 324 against the inner wall or surface 330 of the plate 324 to form a friction lock between the bushing 324 and the plate 314 in a selected polyaxial position. For example, the cancellous screw 370 may be in a first polyaxial position 372 as shown in solid line 372 (see FIG. 11). Alternatively, the cancellous screw 370 may be oriented an angle αα from the first position 372 into a second position 374 as shown in phantom. Alternatively, the cancellous screw 370 maybe positioned in, for example, a third position 376 positioned at an angle ββ from the first position 372. The cancellous screw 370 may thus be positioned with a diverging angle αα or ββ from the first position 372.

Preferably and as shown in FIG. I 1, the proximal portion 340 of the cancellous screw 370 includes external tapered threads 380 which mate with internal threads 382 located within the bushing 324. By providing tapered threads as the cancellous screw 370 is screwed into the bushing 324, the bushing 324 expands with the radially exterior surface 326 of the bushing, seating and securing against the radially interior surface 330 of the plate 314. This provides for stress-free, secure locking of the screw 370 to the plate 314.

Alternatively, the attachment component which mates with the bushing 324 may be provided without any threads in the proximal portion of the attachment component similarly to the screw 170 of FIG. 6A. Such a screw, for example screw 56 of FIG. 6 or screw 980 of FIG. 25, will provide for polyaxial positioning of the attachment component with reduced stress. The screw 56 or 980 unlike cancellous screw 70 (see FIG. 6) does not include threads on the head of screw 56. The lack of screw threads on the head of screw 56 or 980 allows the head to spin on the bushing 24 without locking, thereby achieving a lagging effect.

By positioning the cancellous screw 370 into the first position 372 or the second position 374 or the third position 376, the screw 370 may be positioned to properly secure fragments. For example as shown in FIG. 9 the cancellous screw 370 being positioned in second position 374 may provide for the securing of a fragment CC while the positioning of the cancellous screw 370 in the third position 376 may provide for the securing of fragment DD.

The fracture repair system 310 for use for repairing a fractured tibia may include additional attachment components such as additional attachment component 370. Thus the fracture repair system may include a second cancellous screw 370 positioned at a second plate hole (not shown). In addition to a plurality of cancellous screws 370, the fracture repair system 310 may include, in addition to the polyaxial screws, additional cancellous or cortical screws. For example, Referring to FIGS. 9 and 11, the repair system 310 may include fully threaded cortical screws 352 similar to the cortical screws 52 of the femur plate 14. The cortical screws 352 preferably extend through the cancellous bone 394 and engage with the cortical bone 396. The fracture repair system 310 may further include cancellous screws for example cancellous screws 356 located in the proximal portion 344 of the bone plate 314 as shown in FIG. 11. Such cancellous screws 356 are preferably of a length short enough that they do not reach through to the opposed cortical bone 396. The tibial plate 314 may include one or more tapped openings 360 in the tibial plate 314 which may be utilized to secure a drill guide (not shown), similar to the drill guide 200, for aligning a drill and a screwdriver for the insertion of the screws 352 into the tibia.

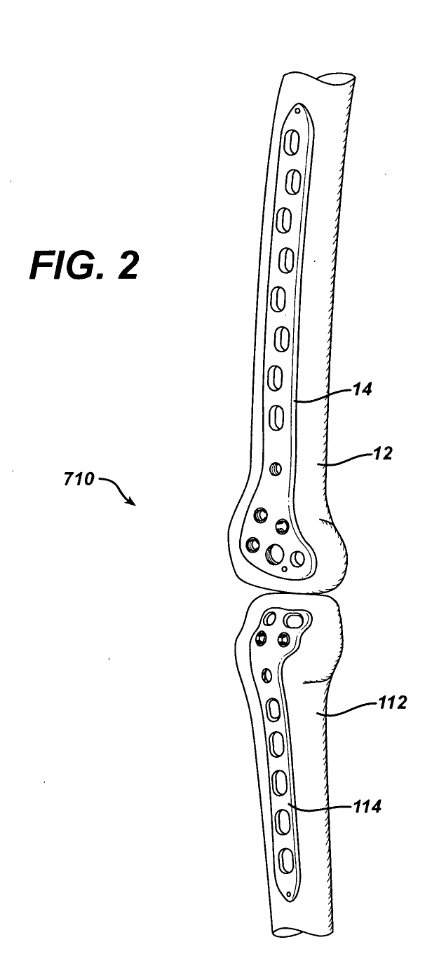

Referring now to FIGS. 1 and 2 a fracture repair system 710 is shown. The fracture repair system 710 comprises an assembly of both a femur plate 14 and a tibia plate 114. Frequently the polyaxial plates of the present invention are sold as a fracture repair system 710 including both a tibial plate 114 and a femur plate 14. Such a combination is often required in severe knee trauma caused, for example, in front-end auto accidents. It should be appreciated that a fracture repair system may include a plate for any other long bone for example a humerus, ulna, fibula or radius.

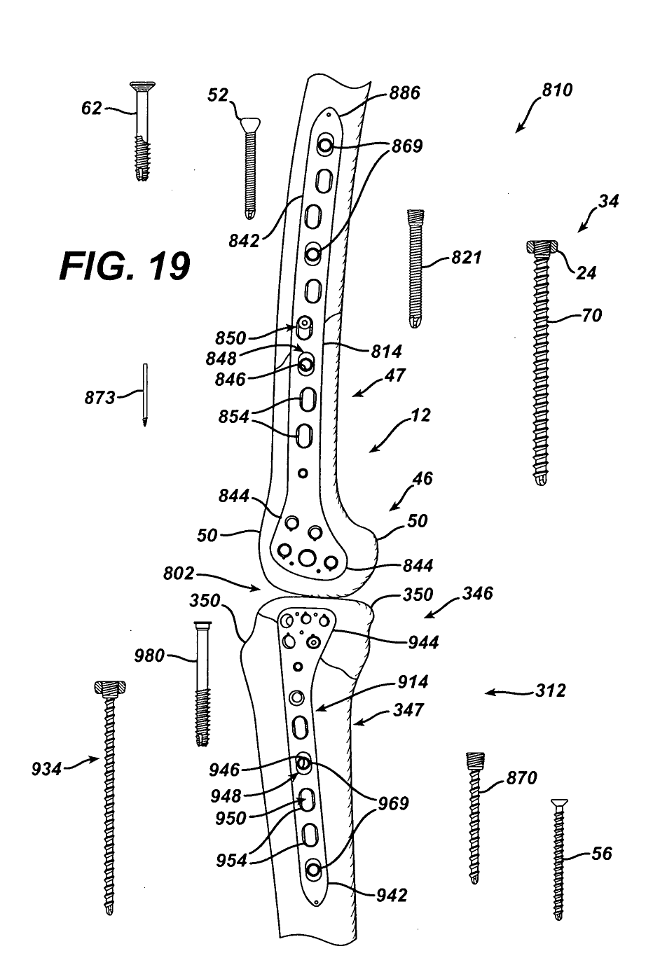

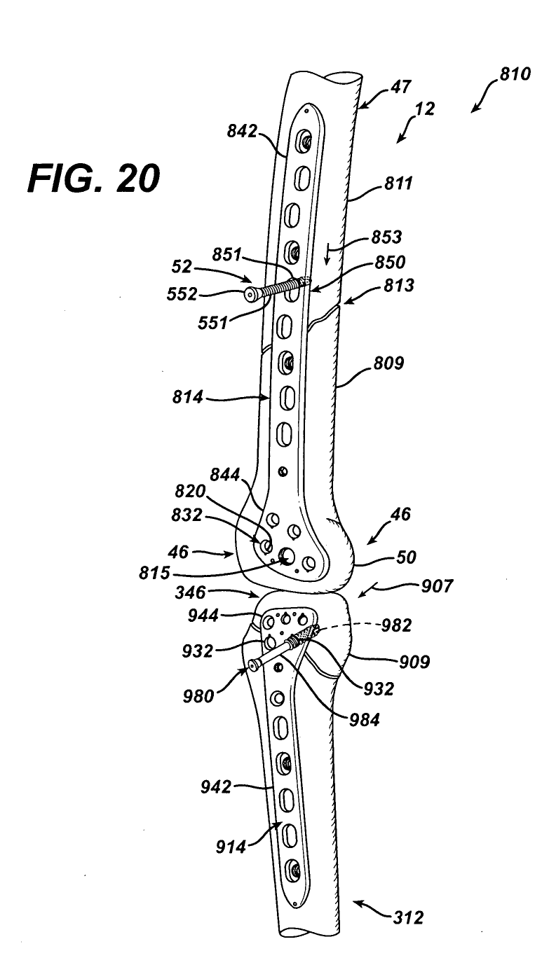

According to the present invention, referring now to FIG. 19 and FIG. 20, another embodiment of the present invention is shown as joint fracture system 810. The joint fracture system 810 is for use with a joint, for example, knee joint 802. The knee joint 802 is associated with adjoining first and second long bone, for example, the femur 12 and the tibia 312. The joint fracture system 810 includes a first plate 814. The first plate 814 cooperates with, for example, the first long bone 12. As shown in FIG. 19, the first long bone 12 may be in the form of a femur. It should be appreciated that the long bone 12 may alternatively be, for example, a tibia, a fibula, a humerus, a radius or an ulna. First plate 812 includes a first plate head portion 844 and a first plate body portion 842. The first plate body portion 842 has an internal wall 846 defining a first plate first body hole 848. The first plate body portion 842 further defines a first plate second body hole 850 spaced from the first plate first body hole 848. Joint fracture system 810 further includes a first plate rigid body attachment component 821, including a stem portion 823 for passage through the first plate first body hole 848 and into the bone 12. The first plate rigid body attachment component 821 further includes an opposed cap portion 825 adapted to rigidly cooperate with the first plate 814 at, for example, the first plate first body hole 848.

The joint fracture system 810 further includes a first plate movable body attachment component in the form of, for example, a solid, fully threaded, cortical screw 52. The first plate movable body attachment component 52 includes a stem portion 551 for passage through the first plate second body hole 850 and into the bone 12. The first plate movable body attachment component 52 further includes an opposed cap portion 552 adapted to movably cooperate with the first plate 814. The screw 52 is shown in greater detail in FIG. 13.

The joint fracture system 810 further includes a second plate 914 for cooperation with the second long bone 312. The second plate 914 may be in the form of for example, a tibia plate and may cooperate with a long bone in the form of, for example, tibia 312. The second plate 914 includes a second plate head portion 944 and a second plate body portion 942. The second plate body portion 942 has an internal wall 946 defining a second plate first body hole 948 and a spaced apart second plate second body hole 950 there through.

The joint fracture system 810 further includes a second plate rigid body attachment component in the form of, for example, attachment component 821. The second plate rigid body attachment component 821 may be identical to the first plate rigid body attachment component 821. Therefore, the second plate rigid body attachment component 821 includes the stem portion 823 for passage through the second plate first body hole 948 and into the bone 312 and the opposed cap portion 825 adapted to rigidly cooperate with the second plate 914.

The joint fracture system 810 may further include a second plate movable body attachment component in the form of, for example, component 52 including the stem portion 551 for passage through the second plate second body hole 950 and into the bone 312 and the opposed cap portion 552 adapted to movably cooperate with the second plate 312.

Referring now to FIG. 20, femur plate 814 is shown in position on long bone or femur 12 and shown for use in repairing a transverse fracture 813. To repair the transverse fracture 813, one end, for example, head portion 844 of the plate 814 is secured to, for example, condylar portion 46 of the femur 12. The head portion 844 may be secured to the condylar portion 46 of femur 12 with, for example, cannulated cancellous screw 62 (see FIG. 12A) which may be secured to large hole 815 in the head portion 844 of the plate 814. A screw, for example, the movable body attachment component, cortical screw 52 (see FIG. 13), is fitted into second hole 850 of the plate 814 with stem 551 of the screw 52 positioned against proximal edge 851 of the hole 850. The screw 52 is then threaded into the bone 12 until the head or cap 552 of the cortical screw 52 contacts the proximal edge 851 of the plate 814.

When the cap 552 of the cortical screw 52 contacts the proximal edge 851 of the plate 814, the plate 814 urges the screw 52 in the direction of arrow 853, which in turn urges first or proximal fragment 811 of the femur 12 in the direction of arrow 853 thereby moving the proximal fragment 811 of femur 12 in contact with second or distal fragment 809 of the femur 12. Thus, the cortical screw 52 cooperating with the plate 814 urges the fragments 811 and 809 into contact with each other. With the fragments 809 and 811 in firm contact with each other, blood flow within the long bone 12 and healing of the fracture site is facilitated.

Continuing to refer to FIG. 20, the use of the present invention to join bone fragments in the condylar portion 346 of long bone 312 is shown. For example, a bone fragment 909 is shown separated from the condylar portion 346 of long bone, for example, tibia 312. A screw, for example, a lag screw such as a partially threaded cancellous screw 980 ( see FIG. 25) may be inserted in, for example, large polyaxial opening 932 in the head portion 944 of the plate 914 and screwed into the condylar portion 346 of the tibia 314. As the screw 980 is advanced in the condylar portion 346 of tibia 312, the screw 980 may contact the fragment 909. As threaded portion 982 of the screw 980 contacts the fragment 909 and as the condylar portion 346 of the tibia 314 is in cooperation with relief portion 984 of the screw 980, the fragment 909 is urged by the screw 980 in the direction of arrow 907 until the fragment 909 moves from its position shown in phantom to the position shown in solid in full contact with the condylar portion 346 of the tibia 314.

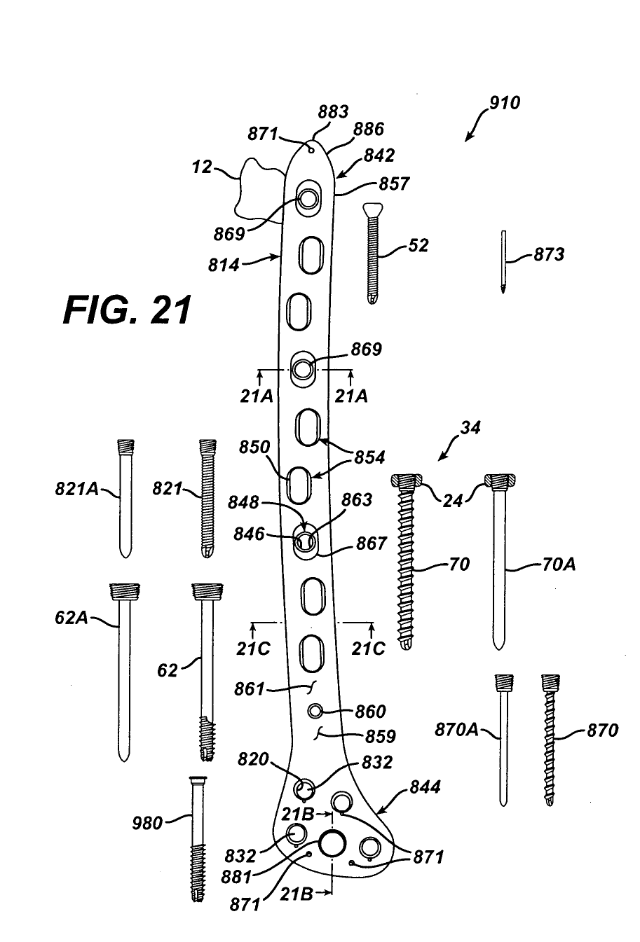

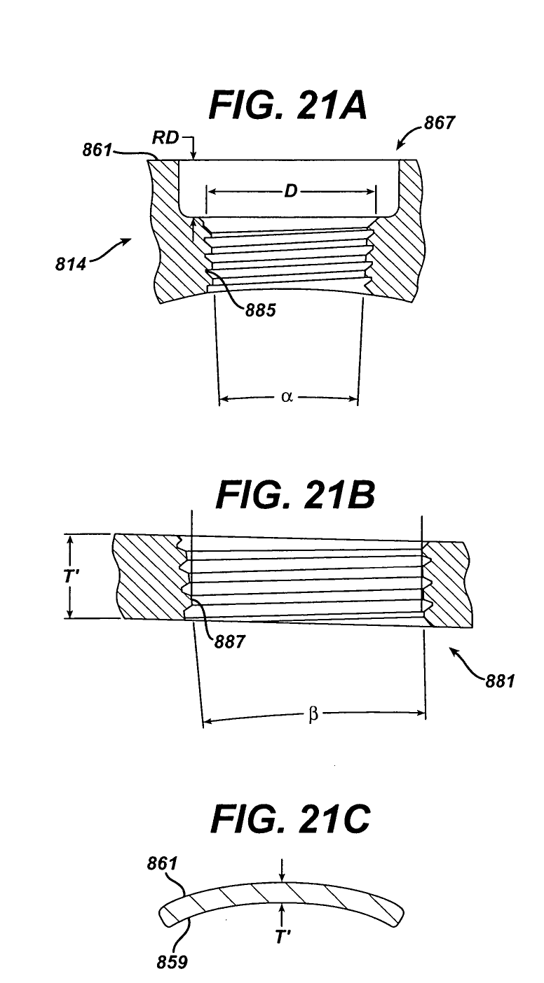

Referring now to FIG. 21, another embodiment of the present invention is shown as fracture repair system 910. The fracture repair system 910 is used for engagement with a bone, for example, the long bone or femur 12. The femur 12 may include a condylar portion 46 and a shaft portion 47 (see FIG. 20). The fracture repair system 910 includes a plate, for example, long bone plate or femur plate 814. The plate 814 includes a head portion, for example, head portion 844 and a body portion, for example, body portion 842. The head portion 844 includes an internal wall 820 which defines a head hole or passageway 832 for the plate 814. The head portion 844 is adapted for cooperation with the condylar portion 46 of the femur 12 (see FIG. 19). The body portion 842 includes internal wall 846 defining body hole 848 through the plate 814.

The fracture repair system 910 further includes a bushing, for example, bushing 24 (see FIG.14). The bushing 24 includes a generally spherically exterior surface 26 adapted for cooperation with the head hole 832 of plate 814. The bushing 24 further includes an opposed interior surface 31 defining a passageway 33 through the bushing 24. The exterior surface 26 of the bushing 24 and the head hole 32 of the plate 814 are configured to permit polyaxial rotation of the bushing 24 within the head hole 832.

The fracture repair system 910 further includes a head attachment component, for example a polyaxial, rigid, cancellous screw assembly such as screw assembly 34 (see FIG. 14). The screw assembly 34 includes a distal portion 36 sized for clearance passage through the passageways 32 and 33 and into the bone 12. The head attachment component 34 further includes an opposed proximal portion 40 sized to urge the bushing 24 against the internal wall 820 of the plate 814 to form a friction lock between the bushing 24 and the plate 814 in a selected polyaxial position. The head attachment component 34 is positionable in an orientation extending divergently from the plate 814.

The fracture repair system 810 further includes a first body attachment component, for example, a rigid cancellous screw such as screw 821 including a stem portion 823 for passage through the first body hole 848 and into the bone 12. The first body attachment component 821 further includes an opposed cap portion 825 sized to cooperate with the plate 814.

It should be appreciated that the plate 814 of the system 910 of FIG. 21 may have a shape and configuration generally similar to that of the femur plate 14 of FIGS. 3, 4 and 5. For example, the plate 814 may have an outer periphery 857 which is substantially the same as outer periphery of the femur plate 14 of FIGS. 3, 4 and 5. As shown in FIG. 21, the plate 814 may define a bone contact surface 859 which closely conforms to the bone or femur 12. The plate 814 may thus have a generally arcuate cross-section as shown in FIG. 21 (c) and have an outer surface 861 which is generally parallel and spaced from the bone contact surface 859. Surfaces 859 and 861 may be spaced apart, for example, a thickness T¹ which may be similar to the thickness T of the plate 14 of FIG. 6.

As shown in FIG. 21, the fracture repair system 910 may include the second body hole 850 through the body portion 842 of the plate 814. The fracture repair system 910 may further include the second body attachment component in the form of, for example, the cortical screw 52 (see FIG. 13). The cortical screw 52 includes the stem portion 551 for passage through the second body hole 850 and into the bone 12 and an opposed cap portion or head 552 sized to cooperate with the plate 814. While the second body hole 850 may have any suitable shape, as shown in FIG. 21, the second body hole 850 may be in the form of an elongated opening 854 similar to the elongated openings 54 of the plate 14 (see FIG. 4).

As shown in FIG. 21, when the plate 814 has a length substantially greater than the width of the plate 814, a plurality of elongated openings 854 may be provided on the body portion 842 of the plate 814. As shown in FIG. 21, the cortical screw 52 and the elongated openings 854 provide for movable attachment of the plate 814 to the bone 12.

The first body hole 848 may have any suitable shape for receiving the first body attachment component or screw 821. For example and as shown in FIG. 21, the screw 821 may be in the form of a screw capable of fixed attachment to the plate 814. The fixable attachment of the screw 821 to the plate 814 may be accomplished by, for example, internal threads 863 formed in wall 846 of the plate 814, which cooperate with external threads 865 formed on cap portion 825 of the screw 821. The internal thread 863 and the external threads 865 may, as shown in FIG. 21, be tapered to provide for rigid locking of the screw 821 to the plate 814.

As shown in FIG. 21, the plate 814 may include elongated recesses 867 positioned centrally about the body hole 848. The elongated recess 867 may, for example, have a shape substantially the same as the elongated openings 854. For example, the elongated openings have a width WF1 and a length LF1 which are substantially the same as the width WF2 and length LF2 of the elongated recesses 867. The elongated openings 854 form a first location feature for cooperating with the drill jig 200 of FIG. 17. Similarly, the elongated recesses 867 define a second location feature for cooperating with the drill jig of FIG. 17. By making the first location feature and the second location feature for the jig 200 be substantially identical in the plate 814, the drill jig 200 for use on the plate 14 of FIGS. 2 through 5 may also be used for the plate 814 of FIG. 21.

While the plate 814 may have a solitary first body hole 848, the plate 814 preferably includes a plurality of the first body holes because the plate 814 has a length substantially greater than its width. For example and as shown in FIG. 21, the plate 814 may include additional threaded body holes 869 which are similar to the first body hole 848.

To permit the plate 814 to be used with body fixed screws 821 and the body movable screws 52, the body portion 842 of the plate 814 may include a pattern of elongated openings 854 and threaded body holes 869. As shown in FIG. 21, the threaded body holes 869 are centrally located along the body portion 842 of the plate 814. Between each adjoining threaded body hole 869, for example, a pair of spaced apart elongated openings 854 are positioned. The plate as shown in FIG. 21 includes six elongated openings 854 and three threaded holes 869 forming a total of nine body mounting holes. To accommodate a wide range of patient femur sizes and shapes, it should be appreciated that the plate 814 may be provided with a different number of body mounting holes. For example, in addition to the nine hole configuration as shown in FIG. 21, the plates may be provided with six, twelve, fifteen or eighteen mounting holes in the body. While the threaded body holes 869 may be centrally positioned on the plate 814, the elongated openings 854 may be offset from the center of the plate and be formed in a staggered position to as shown in FIG. 21 provide a variety of mounting positions for the plate.

To provide for percutaneous installation of the mounting plate 814, the plate 814 may include a threaded mounting opening 860 for mounting the plate 814 to the drill guide 200 (see FIG. 17).

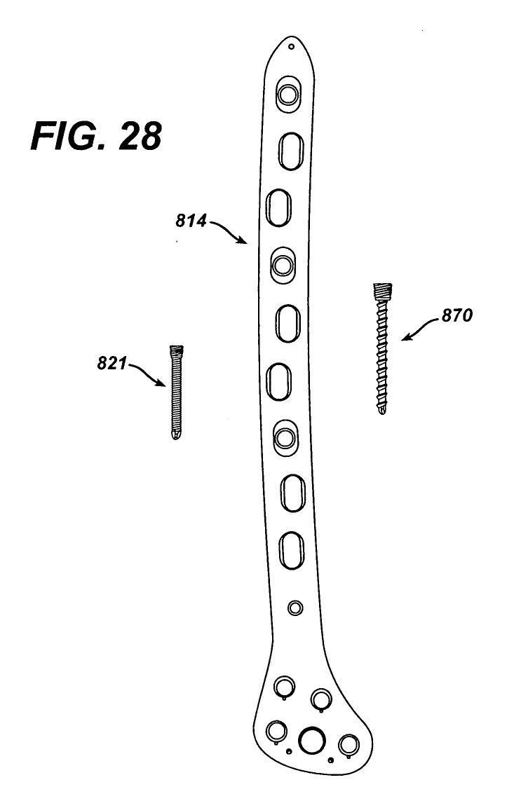

To assist in positioning the plate 814 in a proper position relative to the femur 12, the plate 814 may include a plurality of k-wire holes 871. The k-wire holes 871 are for use with k-wires 873 (see FIG. 28). The plate 814 may be positioned visually over the bone or femur 12 and the k-wires 873 may be installed through k-wire holes 871 into the femur 12. The k-wire holes 871 may be positioned in the head portion 844 and in the proximal portion 842. The K-wire hole 871 in the proximal portion 842 may be used with a suture to move the plate 814 percutaneously along the bone 12.

As shown in FIG. 21, the plate 814 may include a plurality of spaced apart passageways 832 for use with the bushing 24 and the polyaxial cancellous screw 34. For example, as shown in FIG. 21, the plate 814 may include four spaced apart passageways 832.

As can be seen in FIG. 21, the plate 814 may be used with a wide variety of attachment components or screws. It should be appreciated that any connector or fastener which may be fitted into an opening in the plate 814 may be used within the discretion of the surgeon. The plate 814 as shown in FIG. 21 is particularly well suited for the use of particular fasteners or screws in particular openings in the plate 814. For example and as shown in FIG. 21, the elongated openings 854 are particularly well suited for use with the cortical screw 52. It should be appreciated that a lag screw, for example, a partially threaded cancellous screw (not shown) has a threaded stem such as screw 980 (see FIG. 25), may also be used in the elongated openings 854. The lag screw serves to adjoin bone portions from an axial fracture. The lag screw may include a relief portion of stem for clearance passage through the elongated opening 854 and a cap for cooperation with the plate 814.

The threaded body holes 869 are suited particularly for the cortical locking screw 821 (see FIG. 23). The locking screw 821 provides for rigid attachment of the screw 821 to the plate 814. The cortical locking screw 821 is particularly suited for patients with thin-shell or osteoporotic bone.

Occasionally, particularly in osteoporotic bone, bone adjacent the stem portion 823 of the screw 821 may become stripped in the bone 12. A locking cancellous screw 870 is particularly well suited for application in the threaded body holes 869 when the bone adjacent the locking cortical screw 821 is stripped. The screw 821 may be removed from the bone 12 and the screw 870 inserted into the plate 12 in its place. The screw 870 includes a threaded stem portion 873 which may include cancellous screw threads which may be less prone to stripping bone than the cortical threads of the stem portion 873 of the screw 870. As shown in FIG. 21, the threads 865 of the screw 821 may preferably be identical to the threads 877 of the screw 870 so that the screws 821 and 870 are interchangeable at the threaded body holes 869.

Passageways 832 in the head portion 844 of the plate 814 are particularly well suited for use with the attachment component or polyaxial screw assembly 34 (see FIG. 14). The attachment component 34 provides for the polyaxial positioning of the screw assembly 34. The screw assembly 34 may, for example, include the fully threaded cancellous screw 70 including threads for securing cancellous components or fragments of the condylar portion of the long bone. Alternatively, the passageways 832 are also compatible with the lag screw 980 (see FIG. 25). The lag screw 980 is particularly well-suited if the fragments of the condylar portion of the femur 12 are separated and need to be drawn together.

The plate 814 may further include a large threaded head hole 881 for which cannulated cancellous screw 62 (see FIG. 12A) may be used. The cannulated cancellous screw 62 is particularly well suited for joining the fragments in the condylar portion of the long bone 12.

While the arrangement of the elongated openings 854 and the threaded plate body holes 869 may be arranged in any suitable order, the applicants have found that a threaded body hole 869 positioned opposed to the head portion 844, for example, at opposed end 883 of the plate 814 may be preferred. The end 883 of plate 814 will then be rigidly secured to the femur 12 and will avoid movement between the end 883 of the body portion 842 of the plate 814 and the long bone 12 as the patient walks. When all body holes are not used with screws, the end hole is preferably chosen as a screw location to provide stable support for the plate. A threaded body hole adjacent the end 883 permits the end of the plate 814 to be either rigidly or moveably secured.

When utilized for percutaneous installation, the plate 814 of the fracture repair system 910 may include a bullet nose 886. The bullet nose 886 has a bullet or tapered shape to assist in percutaneous insertion of the plate by the implanting surgeon adjacent the femur or long bone 12.

Referring now to FIG. 21 A the threaded body hole 869 of the plate 814 is shown in greater detail. The threaded body holes 869 include internal threads 885 which mate with, for example, threads 865 of the screw 821 (see FIG. 23). Threads 885 may be tapered and defined by an included angle α. The threaded hole 869 has a diameter D selected to mate with the cap portion 825 of the screw 821 (see FIG. 23). The threaded body hole 869 is in alignment with the elongated recess 867. The elongated recess 867 is recessed a distance RD from outer surface 861 of the plate 814. In order to provide to sufficient strength in the threads 885, the threads 885 may, for example, be triple lead threads. In a triple lead thread, the screw advances axially as it is rotated three times as far as the distance between adjacent threads. By utilizing the triple lead threads for threads 855 while maintaining a single lead thread on the stem portion of the screw, for example, stem 823 of the screw 821 (see FIG. 23), a coarse cancellous thread may be used on the screw stem 823 and strong, fine threads may be used in the holes of the plate 814.

Referring now to FIG. 21 B, the large threaded hole 881 is shown in greater detail. The large threaded hole 881 may include threads 887 which may be tapered and defined by an included angle β. The threads 887 like the threads 885 may be triple lead threads to provide for a strong thread in the plate 814 and in the cap portion of the screw while providing a coarse thread in the stem portion of the screw.

The plate 814 may be made of any suitable durable material that is biologically compatible with the human anatomy and preferable made of a high strength metal. For example, the plate may be made of stainless steel, cobalt chrome or titanium. Preferably the plate 814 is manufactured from a forged or wrought titanium alloy. One such suitable alloy is ASTM F-620-97 and another suitable alloy is ASTM F-136 ELI.

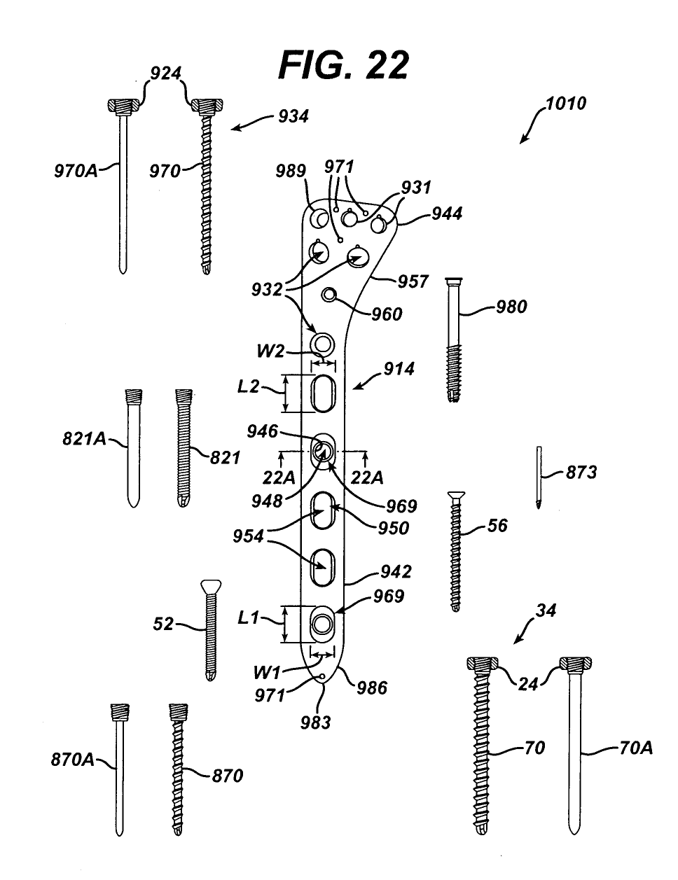

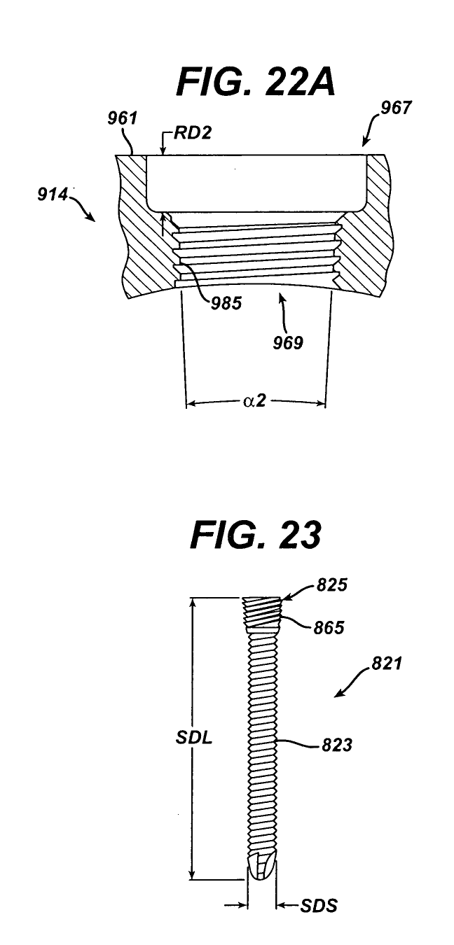

Referring now to FIG. 22, another embodiment of the present invention is shown as fracture repair system 1010. The fracture repair system 1010 is for use for engagement with a bone, for example a tibia 312 having a condylar portion 346 and a shaft portion 347 (see FIG. 19). The fracture repair system 1010 includes a plate in the form of for example a tibia plate 914. The tibia 914 may have any suitable size and shape and includes a head portion 944 and a body portion 942. The body portion 942 has an internal wall 946 defining a first body hole 948. The body portion 942 further includes a spaced apart second body hole 950 through the body portion 942.

The fracture repair system 1010 further includes a rigid body attachment component in the form of, for example, rigid cortical screw 821 (see FIG. 23) including a stem portion 823 for passage through the body hole 948 and into the bone 312 and an opposed cap portion 825 adapted to rigidly cooperate with the plate 814. The fracture repair system 1010 further includes a movable body attachment component in the form of, for example, moveable cortical screw 52 (see FIG. 13) including the stem portion 551 for passage through the second body hole 950 and into the bone 312. The movable body attachment component 551 further includes an opposed cap portion 552 adapted to movably cooperate with the plate 914.

The fracture repair system 1010 of FIG. 22 is particularly well adapted for use with bone when the bone is, for example, a tibia 312. The tibia 312 may either be osteoporotic or healthy bone. The choice of the use of locking and non-locking plate construction may depend on whether the bone is osteoporotic or healthy. If, for example, the bone is osteoporotic, a rigid attachment of the screws to the plate may be preferred. In such a rigid attachment, the rigid body attachment components in the form of, for example, rigid cancellous screw 821 (see FIG. 23) are utilized in the hole 948. Conversely, if the bone is not osteoporotic, the movable body attachment components, for example movable cortical screws 52 are utilized in the hole 950 for movable attachment with the plate 914.

While the plate 914 may have any suitable shape for cooperation with the long bone, for example, the tibia, the plate 914 may have an outer periphery 957 similar to the periphery of the tibia plate 314 of FIGS. 7, 8 and 9. The plate 914 may include additional threaded body holes 969 similar to the hole 948. Similarly, the plate 914 may include additional clearance holes similar to the hole 950 in the form of, for example, elongated openings 954. Similar to the configuration of the femur plate 814, the tibia plate 914 may include elongated recesses 967 centrally located around the threaded body holes 967. The elongated recesses 967 have a width W1 and a length L1 preferably identical to the width W2 and the length L2 of the elongated openings 954. The elongated openings 954 and the elongated recesses 967 are preferably sized for compatibility with the drill guide 200 of FIG. 17.

Elongated opening 954 and the elongated recesses 969 may, as shown in FIG. 22, be centrally located along the length of the body 942. As shown in FIG. 22, two elongated openings 954 may be positioned between each threaded body hole 969. As shown in FIG. 22, two threaded body holes 969 and three elongated opening 954 representing a total of five openings are shown. The plate 914 may include any multiple of three openings, for example, 8, 11 or 14 openings.

Similarly to the plate 814 of FIG. 21, the plate 914 may include one of the threaded location holes 969 positioned at end 983 of the plate 914 to rigidly secure the end of 983 to the bone 312 and to prevent motion between the end 983 and the bone 312 as the patient walks. Further, since the end hole should be used whenever possible, even if all holes are not used, the use of a threaded body hole adjacent the end 883 permits the end of the plate to be either rigidly or moveably secured. Similarly, the plate 914 may include a bullet nose 986 similar to the bullet nose 886 of the plate 914. The plate 914 may include a threaded guide hole 960 for cooperation with the drill guide 200.

The plate 914 may further include a plurality of k-wire holes 971 for cooperation with the k-wire 973 of FIG. 28. As shown in FIG. 22, the plate 914 may include three k-wire holes 971 in the head 944 and one k-wire hole 971 adjacent the end 983 of the plate 914. The plate 914 may further include large passageways 932 similar to the passageways 832 of the plate 814 of FIG. 19 for cooperation with the attachment component, for example, screw assembly 34 (see FIG. 14). The attachment component 34 provides for polyaxial location of the cannulated cancellous screws 70.

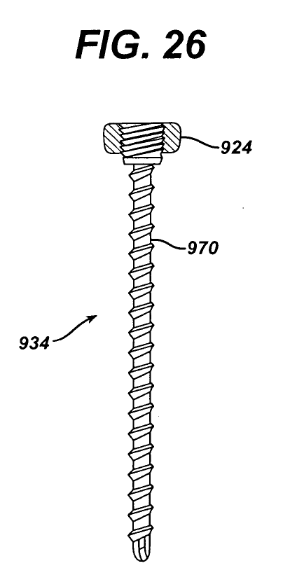

The plate 914 may further include small passageways 931 smaller than the passageways 932 for cooperation with polyaxial attachment component 934. The polyaxial attachment component 934 (see FIG. 26) is similar but smaller than the polyaxial attachment component 34 of FIG. 14.

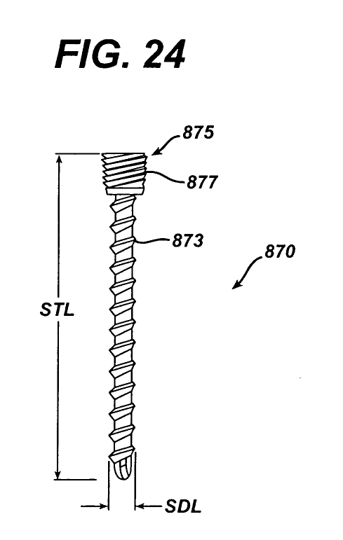

While it should be appreciated that any fastener which may fit in an opening in the plate may be utilized therewith, the plate 914 of FIG. 22 includes holes which are designed for use with particular fasteners. For example, as shown in FIG. 22, the elongated openings 954 are compatible with the cortical screws 52 of FIG. 13. It should be appreciated that the lag screw (not shown) having a stem like that of screw 980 of FIG. 25 may similarly be put in the elongated openings 954. The lag screw may be used to adjoin portions of bone in an axial fracture. The threaded body openings 969 are compatible with the body attachment component or screw 821 of FIG. 23 and with the cancellous screw 870 of FIG. 24. The screws 821 and 870 are chosen for use with plate 984 for the same reason that they are chosen for use with plate 814 of FIG. 21. The passageways 932 are compatible with screw assembly 34 including cancellous screw 70 of FIG. 14 as well as with the lag screw 890 of FIG. 25. The small passageways 931 are compatible with screw assembly 934 including screw 970 (see FIG. 26).

As shown in FIG. 22, the plate 914 may further include a small screw opening 989 located in the head portion 944 of the plate 914. The small screw opening 989 is designed for use with fully threaded cancellous screw 56 of FIG. 18.

Referring now to FIG. 22A the threaded body opening 969 is shown in greater detail. The threaded body opening 969 includes internal threads 985 which may, as shown in FIG. 22A, be similar to the internal threads 885 of the threaded body opening 869 of FIG. 21A and thus may be of a triple lead type. The threaded body opening 969 is centrally positioned with respect to the elongated recess 967. The elongated recess 967 is recessed from the surface 961 of the plate 914 a distance of RD2 which may be the same as distance RD of FIG. 21A. The threads 985 may be tapered and defined by an included angle α2 which may be identical to the angle α of the threads 885 of the plate 814 of FIG. 21A.

The plate 914 may be made of any suitable durable material that is biologically compatible with the human anatomy and preferable made of a high strength metal. For example, the plate may be made of stainless steel, cobalt chrome or titanium. Preferably the plate 914 is manufactured from a forged or wrought titanium alloy. One such suitable alloy is ASTM F-620-97 and another suitable alloy is ASTM F-136 ELI.

Referring now to FIG. 23, the screw 821 is shown in greater detail. The screw 821 as shown in FIG. 23 is a fully threaded cortical type screw with a fixed locking style. The screw 821 includes a stem 823 including cortical threads that extend to the cap 825 of the screw 821. The cap 825 includes tapered triple lead threads 865. As shown in FIG. 23, the screw 821 is self-tapping. Screw 821 may have various lengths SDL, for example, from 14 mm to 40 mm and may have a stem diameter SDS of, for example, 4.5 mm and a cap diameter of, for example, 5.5 mm.

Referring now to FIG. 24, the screw 870 is shown in greater detail. The screw 870 is similar to cancellous screw 70 of FIG. 14 and differs from the cancellous screw 70 of FIG. 14 only in its overall length. The screw 870 has, for example, a stem 873 having fully threaded cancellous threads. Screw 870 further includes a cap 875 having tapered external threads 877 similar to the threads 865 of the screw 821 (see FIG. 23) and therefore includes screw threads that are triple lead. The screw 870 may have a length SCL of, for example, 25mm to 100 mm and may have a stem diameter SDL of, for example, 5.5 mm as well as a cap diameter of 5.5 mm.

Referring now to FIG. 25, lag screw 980 is shown in greater detail. Lag screw 980 includes a partially threaded stem 843 including a relief portion 984 and a threaded portion 982. The threaded portion 982 includes cancellous threads. The screw 841 further includes a cap 845. The stem 843 may have a diameter DL of, for example, 5.5 mm and may have a length LL, of, for example, 50 mm to 100 mm.

Referring now to FIG. 26, polyaxial cancellous screw assembly 934 is shown in greater detail. The screw assembly 934 includes screw 970 and bushing 924. The screw 970 is similar to the cancellous screw 70 of FIG. 14. The bushing 924 is similar to the bushing 24 of the screw assembly 34. Screw 970 is smaller than the screw 70 of FIG. 14.

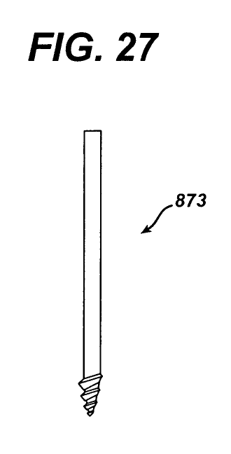

Referring now to FIG. 27, a k-wire 873 is shown. The k-wire 873 is suitable for use with the k-wire holes 871 and 971 of the plates 814 and 914, respectively. The k-wire has a generally cylindrical shaped body 893 with a cutting tip 895 located on an end thereof.

Referring now to FIG. 28, another embodiment of the present invention is shown as fracture repair system 1110. Fracture repair system 1110 is utilized for engagement with a bone, for example a femur 12 having a condylar portion 46 and a shaft portion 47 (see FIG. 19). The system 1110 includes a plate, for example, plate 814 including a head portion 844 and a body portion 842. The body portion 842 has an internal wall 846 defining a body hole 848 through the plate 814. The fracture repair system 1110 further includes a first rigid body attachment component 821, including a stem portion 823 for clearance passage through the body hole 848 and into the bone 12. The first rigid body attachment component, for example, rigid cortical screw 821 (see FIG. 23) further includes opposed cap portion 825 adapted to rigidly cooperate with the plate 814. The fracture repair system 1110 further includes a second rigid body attachment component 870 (see FIG. 24) including stem portion 870 for threadably engagement with the body hole 875 and into the bone 12 and an opposed cap portion 875 adapted to rigidly cooperate with the plate 814.

Fracture repair system 1110 provides for the use of the second fastener 870 when the first fastener 821 is stripped. Therefore, the stem 873 has a stem diameter SDL which is preferably larger than the stem diameter SDS of the stem 823 and in fact the stem 853 may be made of a coarse thread or a cancellous thread while the stem 873 may be a fine or cortical type thread.

Referring again to FIGS. 21 and 22, the fracture repair system of the present invention may alternatively utilize pins to replace at least a portion of the threaded fasteners of the repair system. Preferably the pins are secured to the plates 814 and 914 of FIGS. 21 and 22, respectively. Thus the pins are preferably used to replace the threaded fasters that have tapered threads that engage and lock to the plates 814 and 914. The pin may, for example have dimensions that are the same as the respective screw they replace including the same length and head configuration. The pins may have tapered threads adjacent the heads for securing the pin to the plate that are the same as the tapered threads of the respective screw. The pins may have a periphery on the pin shank that does not contain threads.

The diameter of the pin shank may be any diameter sufficient for proper strength. For example, the diameter of the pin shank may be the same as the respective screw thread major diameter or the minor diameter or, for example, any size in between. If a pin is used with the same diameter as the minor diameter of the respective screw the same drill may be used to prepare the pin as is used to prepare the hole for the screw. Also, a pin with a diameter equal to the minor diameter of the screw would have about the same strength as the screw, but be less invasive to the bone around where the pin is inserted than the respective screw.

For example and as shown in FIG. 21, the pin 821A may be used to replace the screw 821 and the pin 70A may be used to replace the screw 70. Further the pin 870A may be used to replace the screw 870 and the pin 62A may be used to replace the screw 62.

Further, as shown in FIG. 22, the pin 934A may be used to replace the screw 934.

The pins may be installed by first preparing an opening in the bone for receiving the pin. A drill (not shown) may prepare the opening and a bushing (not shown) may be positioned over the hole in the plate to guide the drill. The drill may have the same diameter as the pin. The pin may be pushed into the drilled opening by any suitable method.

The pins may provide support for the plate in the longitudinal axis of the bone, transverse to the longitudinal axis of the pin. The pins may be easier to install than screws and may be less disruptive to the bone adjacent where they are installed.

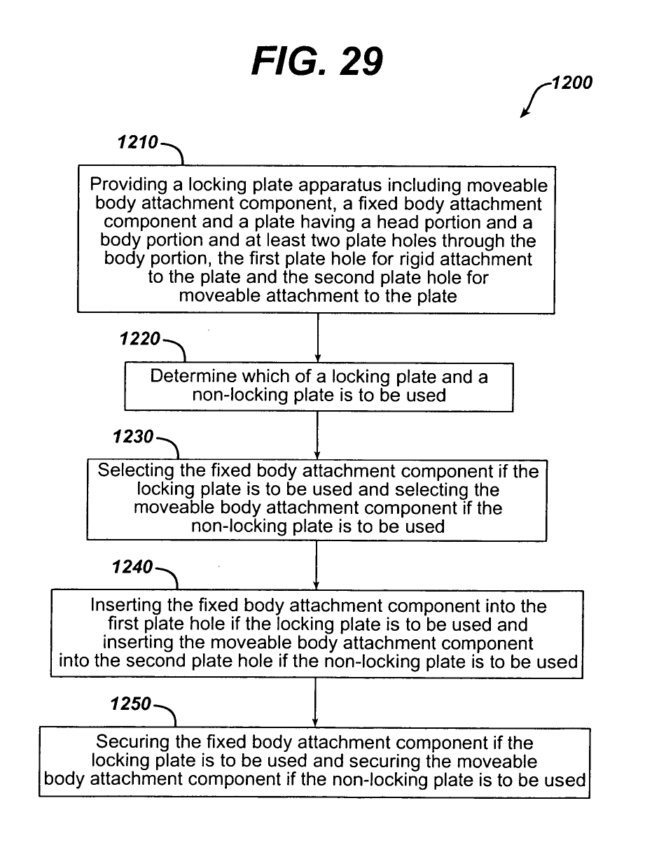

Referring now to FIG. 29, another embodiment of the present invention is shown as method 1200. The method 1200 is utilized for repairing a bone fracture on a bone having a condylar portion and a shaft portion. The method 1200 includes a first step 1210 of providing a locking plate apparatus including movable body attachment component, a fixed body attachment component and a plate. The plate includes a head portion and a body portion and at least two plate holes through the body portion, the first plate hole for rigid attachment to the plate and the second plate hole for movable attachment to the plate. The method further includes a second step 1220 of determining which of a locking or non-locking plate bone is to be used. The method 1200 also includes a third step 1230 of selecting the fixed body attachment component if a locking plate is to be used and selecting the movable body attachment component if a non-locking plate is to be used. The method 1200 further includes a fourth step 1240 of inserting the fixed body attachment component into the first plate hole if the locking plate is to be used and inserting the movable body attachment component into the second plate hole if the non-locking plate is to be used. The method includes a fifth step 1250 of securing the fixed body attachment component if the locking plate is to be used and securing the movable body attachment component if the non-locking plate is to be used.

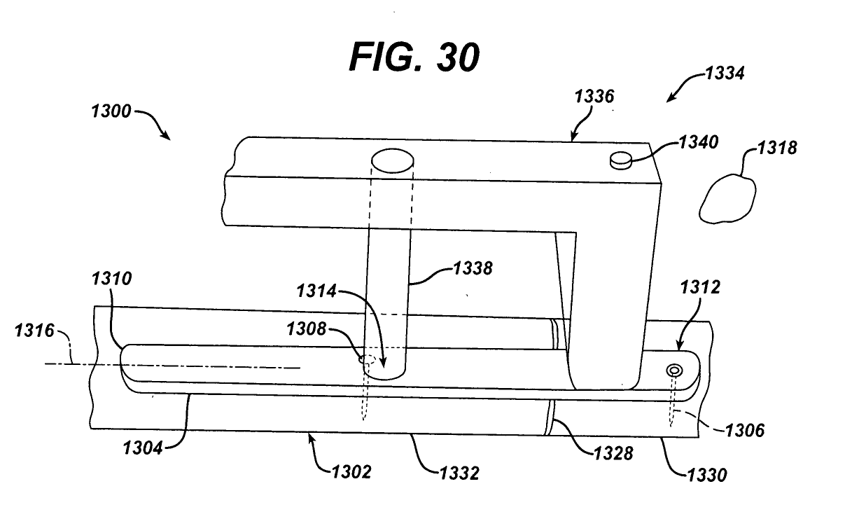

According to the present invention and referring now to FIG. 30, a system 1300 is shown for percutaneous fracture repair of bone 1302. The system 1300 includes a plate 1304, a first attachment component 1306 and a second attachment component 1308. The plate 1304 may be any plate which is compatible with the bone 1302. The bone 1302 may be any bone and may, for example, be a long bone, for example a femur, tibia, fibula, humerus, ulna or radius. The plate 1304 may be adaptable for use in percutaneous bone repair. For example, the plate 1304 may include a nose 1310 located on an end of the plate 1304 for assisting in the percutaneous insertion of the plate 1304. The plate 1304 has a first feature 1312 and a second feature 1314. The first feature 1312 is spaced from the second feature 1314. The plate 1304 defines a longitudinal axis 1316 thereof.

The first attachment component 1306 is operably associated with the first feature 1312. The first attachment component 1306 is adapted to cooperate with the bone 1302.

The second attachment component 1308 is similarly operably associated with the second feature 1314. The second attachment component 1308 is percutaneously inserted into the second feature 1314. The second attachment component 1308 is operably associated with the plate 1304 to provide a compressive force in the bone. The compressive force has a component thereof in the longitudinal axis 1316. The second attachment component 1308 is adapted for cooperation with the bone 1302.

While as shown in FIG. 30, the second attachment component 1308 is inserted percutaneously. It should be appreciated that to minimize the incision for the patient, the plate 1304 may likewise be inserted percutaneously at, for example, incision site 1318.

The first attachment component 1306 may, similarly to the second attachment component 1308, be percutaneously inserted. Percutaneous insertion of the first attachment component 1306 may not be advantages when the first attachment component 1306 is in alignment with the incision site 1318 where the plate 1306 is inserted. In such cases the first attachment component 6 may be inserted by open surgical procedures.

It should be appreciated that the first feature 1312 and the second feature 1314 may be any feature in the plate 1304 which can cooperate with the attachment components 1306 and 1308, respectively. For example, the features 1312 and 1314 may be in the form of protrusions, grooves, slots, and recesses. The features 1312 and 1314 may as shown in FIG. 30 be in the form of openings. The openings may be threaded or unthreaded. The first attachment component 1306 and the second attachment component 1308 may be any attachment component which may be utilized to secure the plate 1304 to the bone 1302. For example, the first attachment component 1306 and the second attachment component 1308 may be in the form of a screw or a pin.

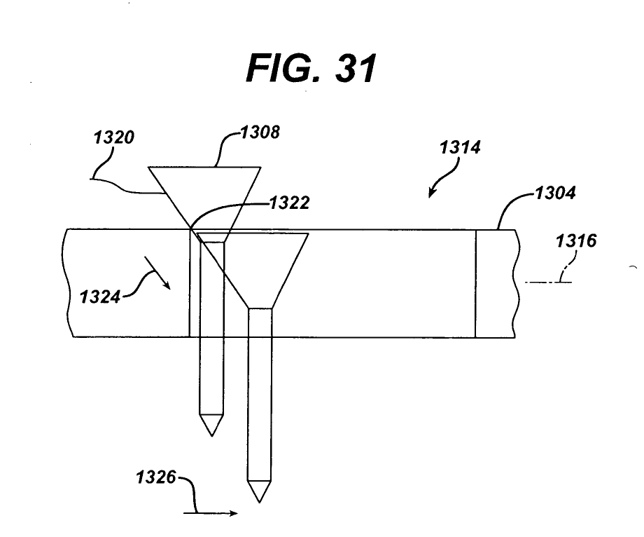

For example and as shown in FIG. 30 the system 1300 may be utilized by first securing the first attachment component 1306 to the bone 1302 and thereby securing the plate 1304 to the bone 1302. The second attachment component 1308 is then secured to the bone 1302 thereby securing the plate 1304 between the screw 1308 and the bone 1302. To provide compression to the bone 1302, for example and as shown in FIG. 31, the second attachment component 1308 defines a first surface 1320 of the second attachment component 1308. The second feature or opening 1314 of the plate 1304 defines a first surface 1322 of the opening 1314. The first surface 1320 of the second attachment component 1308 cooperates with the first surface 1322 of the second opening 1314 of the plate 1304 as the second attachment component 1308 advances toward the bone 1302 to compress the bone 1302. As the first surface 1320 and the first surface 1322 cooperate in the direction of arrow 1324, the screw or second attachment component 1308 advances in the direction of arrow 1326 to provide a compressive force along the longitudinal axis 1316 of the plate 1304.

As shown in FIG. 30 and 31, the bone 1302 defines a fracture 1328 therethrough. The fracture 1328 may separate the bone 1302 into a first bone portion 1330 and a spaced apart second portion 1332. The first attachment component 1306 and the first feature 1312 are associated with the first bone portion 1328 while the second attachment component 1308 and the second feature 1314 are associated with the second bone portion 1332.

The second attachment component 1308 may be percutaneously inserted into the patient in any suitable fashion. For example, the second attachment component 1308 may be percutaneously inserted by first providing a hollow tube or trocar in alignment over the second feature or opening 1314 in the plate 1304. The second attachment component 1308 may then applied to the bone 1302 through the opening 1314 by passing the second attachment component 1308 through the trocar (not shown).

Preferably, however, to provide for a proper positioning of a trocar or tube for percutaneously inserting the second attachment component 1308 into the bone 1302, a guide 1334 may be provided to assist in the percutaneous installation of the second attachment component 1308. The guide 1334 serves to guide the second attachment component 1308 into engagement with the second feature or opening 1314 and into the bone 1302.

While the guide 1334 may have any suitable size and shape, the guide 1334 as shown in FIG. 30 includes a body 1336 attachable to the plate 1304 and a tube 1338. The tube 1338 extends from the body 1336. The tube 1338 is utilized for guiding the second attachment component 1308 into cooperation with the second feature 1314. It should be appreciated that the tube 1338 may be a separate component from the body 1336 or alternatively may be integral with the body 1336.

The guide 1334 may be secured to, for example, the plate 1304. For example, the body 1336 may be secured to the plate 1304 by a fastener 1340 securing the body 1336 to the plate 1304. The guide 1334, the plate 1304 and the tube 1338, as well as the fasteners 1306 and 1308 may be made of any suitable, durable material such as polymer or a metal. If made of such a metal, the metal may be a cobalt chromium alloy, a titanium alloy or a stainless steel alloy.

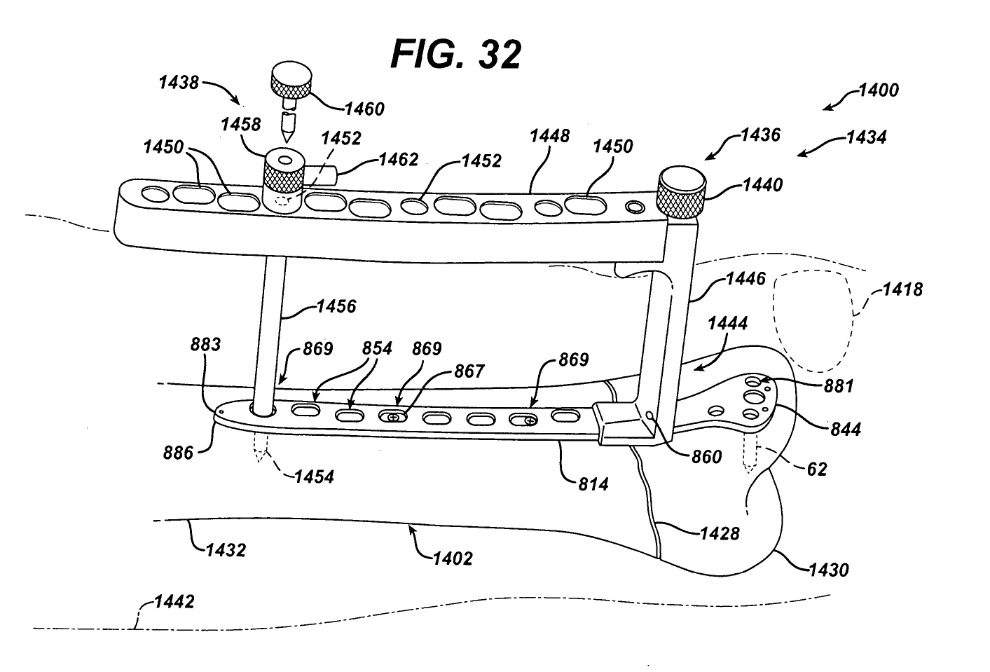

Referring now to FIG. 32, another embodiment of the present invention is shown as fracture repair system 1400. Fracture repair system 1400 is similar to fracture repair system 1300 of FIGS. 30 and 31 and provides for applying a compressive force to the bone during the plating process and for providing a percutaneous insertion of an attachment component. The system 1400 is shown including a plate in the form of a femoral plate 814. It should be appreciated the system 1400 may be used with tibial plates, ulnar plates, or humeral plates. It should also be appreciated that system 1400 may be used with any plate used for fracture repair in the body. The system 1400 is utilized to provided a percutaneous fracture repair of bone 1402.

When utilizing the system 1400, the plate, for example plate 814, is placed under the skin through incision site 1418 and into position on the surface of long bone or femur 1402. The incision site 1418 in the thigh 1442 is, as shown in FIG. 32, large enough to permit the insertion of the plate 814. The site 1418, as shown in FIG. 32, may be large enough to expose the first feature in the form of a large opening 881 of the plate 814 to permit traditional plating methods to be used to insert the first attachment component in the form of, for example, cortical screw 62 into the large opening 881. At this point, the plate 814 is secured at end portion 844 of the plate 814.

As shown in FIG. 32, the plate 814 is inserted through the incision site 1418 in the direction of arrow 1444 along the femur 1402 until it rests in the position as shown in FIG. 32. The movement of the plate 814 in the direction of arrow 1444 provides for a percutaneous installation of the plate 814 against the femur 1402.

Since the plate 814 is positioned percutaneously, it is important that the plate 814 be properly aligned with respect to the long bone or femur 1402. Preferably, and as shown in FIG. 32, a guide 1434 is utilized to assist in the proper positioning of the plate 814 as well as to provide percutaneous installation of an attachment component for attaching the plate 814 to the femur 1402.

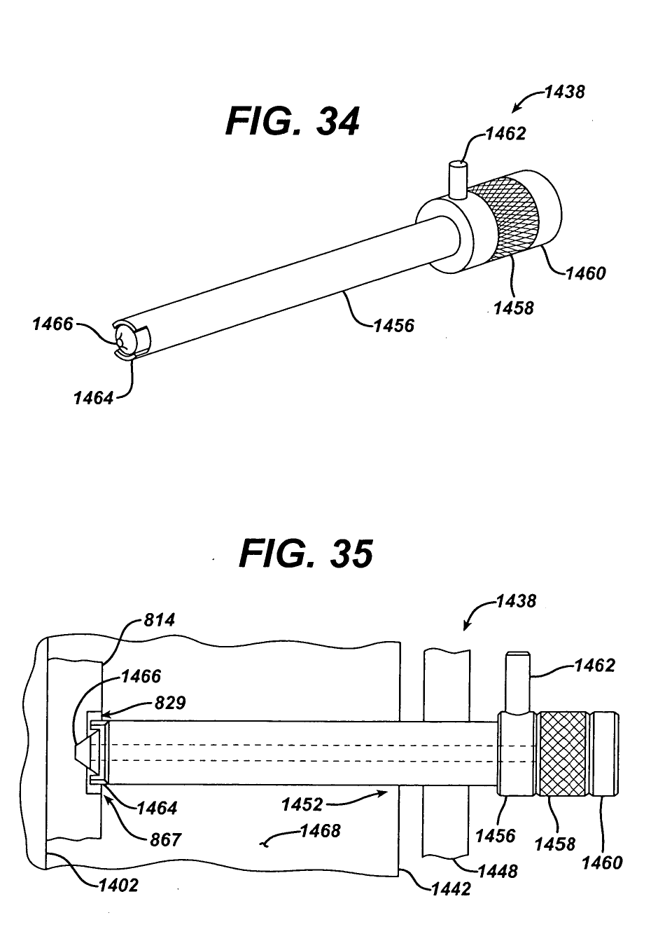

The guide 1434 may be any guide suitable for assisting and positioning the plate 814 and for providing percutaneous installation of attachment components. For example, and as shown in FIG. 34, the guide 1434 includes a body 1436 from which tube 1438 extends. The body 1436 and tube 1438 may be integral with each other or may be, as shown in FIG. 32, separate components. In fact, the body 1436 may, in fact, be modular or be made of several separate components which are secured together. For example, and as shown in FIG. 32, the body 1436 may include a riser or handle 1446 connectable to the plate 814 and a targeting guide 1448 mounted on the handle 1446. A connecting screw 1440 may be utilized to connect the handle 1446 and the guide 1448 to the plate 814.

By providing the modular construction, it should be appreciated that an identical targeting guide 1448 may be utilized with a series of different handles with a different, for example, height for providing a variety of spacings between the targeting guide 1448 and the plate 814. Such variations in height may be necessary to accommodate heavier patients. The modular construction of the guide 1434 may further provide for a plurality of targeting guides to accommodate plates with varying lengths and shapes. Further the targeting guide 1448 shown in FIG. 32 may be rotated 180 degrees around its longitudinal axis and utilized to accommodate both right hand and a left hand arcuate targeting guides.

While installing the plate 814 and the guide 1434, the alignment of the plate 814 with respect to the femur 1404 is difficult percutaneously. The guide 1434, in particularly the targeting guide 1448 of the guide 1434, serves as a visual aid in properly positioning the plate 814 against the femur 1404.

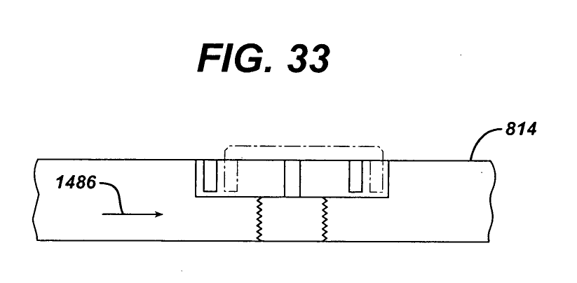

The system 1400 may be utilized with the guide 1434 anchored securely to the plate 814 and the femur 1402 as shown in FIGS. 32-39. For example, the first cortical screw 62 may be positioned in the plate 814 and may be used to secure the end of the plate 814 against the femur 1402. After the screw 62 is secured, the opposed end 883 of the plate 814 may be secured to provide for an accurate positioning of the plate 814 and to provide support to stabilize the targeting guide 1448. For example, and as shown in FIG. 32, a tube 1438 may be secured to the targeting guide 1448 and used to stabilize the guide 1434 against the plate 814. The tube 1438 may be utilized to prepare the femur 1402 for receiving a stabilizing or anchoring device to anchor and stabilize the targeting guide 1448.

As shown in FIG. 32, the targeting guide 1448 may include a plurality of openings including, for example a pattern of elongated openings 1450 and cylindrical openings 1452. The openings 1450 and 1452 may form a pattern corresponding in shape and alignment with the elongated openings 854 and the threaded body holes 869 of the plate 814, respectively.

The cortical screw 62 may be utilized to secure the end portion 844 of the plate 814 to, for example, the first bone portion 1430 of the femur 1402. A transverse fracture 1428 may, for example, separate the first bone portion 1430 from second bone portion 1432. The end 883 of the plate 814 may be secured to the second bone portion 1432 at, for example, its most distal possible location, for example, at threaded body hole 869.

As shown in FIG. 32, the tube 1438 may be used to prepare an anchor hole 1454 for anchoring the guide 1434 to the plate 814 and the femur 1402. The anchor hole 1454 is preferably, as shown in FIG. 32, prepared percutaneously. The tube 1438 may, as shown in FIG. 32, be modular or include a plurality of components. For example, the tube 1438 may include a round sheath 1456 which may, for example, fit slidably within the cylindrical opening 1452 of the targeting guide 1448 and seat against, for example, threaded body hole 869 of the plate 814. The round sheath 1456 may be cannulated or hollow and the tube 1438 may further include a drill bushing 1458 which slidably fits within the round sheath 1456. A solid pin 1460 may slidably fit within the drill bushing 1458.

Referring now to FIGS. 34 and 35, the tube assembly 1438 is shown in greater detail. The tube assembly 1438 may include the round sheath 1456 which slidably fits in the cylindrical opening 1452 of the targeting guide 1448. The round sheath 1456 may include the orientation feature 1462 for visually angularly orientating the round sheath 1456 so that the sheath 1456 may be properly positioned with respect to the plate 814.

Referring again to FIGS. 34 and 35, the round sheath 1456 includes the orientation feature 1462 in the form of, for example, transverse pin 1462. The transverse pin 1462 is utilized, for example, for angularly positioning the round sheath 1456 with respect to the plate 814. Round sheath 1456, as shown in FIGS. 34 and 35, may include an alignment protrusion 1464 for aligning and positioning the round sheath 1456 with respect to the plate 814. The alignment protrusion 1464 may cooperate with, for example, a plate feature 829 in the form of, for example, elongated recess 867. To permit percutaneous compression of the plate 814, the tube assembly 1438 including the round sheath 1456, bushing 1458 and pin 1460 are inserted through cylindrical opening 1452 of the targeting guide 1442 and point 1466 of the pin 1460 is urged through soft tissue 1468 of the thigh 1442 until the tube assembly 1438 is in position resting against the elongated recess 867 of the plate 814.

After the tube assembly 1438 is placed in position against the plate 814, the pin 1460 may be removed from the bushing 1458 of the tube assembly 1438. The tube assembly 1438 provides increased stability and strength to the guide 1434. The tube assembly 1438 also provides for accurate positioning of the targeting guide 1448 with respect to the plate 814.

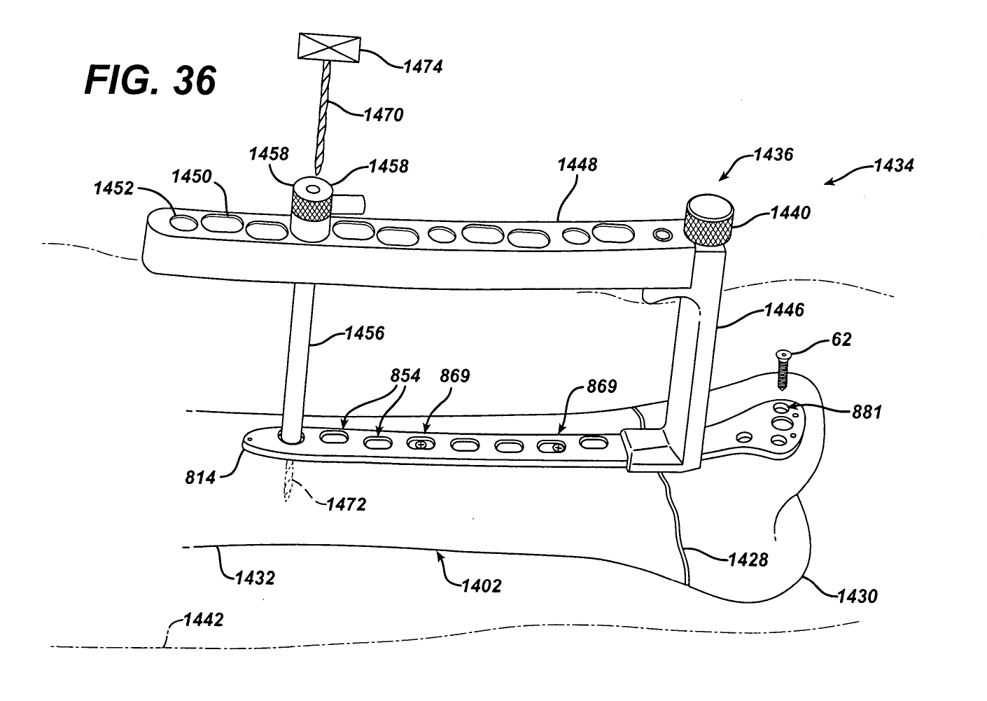

Referring now to FIG. 36, a drill 1470 may be utilized to prepare a hole 1472 in the femur 1402. The drill 1470 may be used to prepare the hole 1472 using any commercialized power drill 1474, for example an electric, pneumatic or battery powered drill.

It should be appreciated that the handle 1446 and the targeting guide 1448 may be utilized to manually position the plate 814 in its proper orientation with respect to the femur 1402. The targeting guide 1448 is preferably visually positioned with respect to the thigh 1442.

While the targeting guide 1448 may be made of any suitable durable material capable of being sterilized by existing sterilizing techniques such as autoclaving, the targeting guide 1448 may be made of a radio-translucent material. If the targeting guide 1448 is made of radio-translucent material, for example a polymer, the patient may be x-rayed during the procedure so that the proper orientation of the plate 814 with respect to the femur 1402 may be assured prior to the drilling of the hole 1472 in the femur 1402.

Referring now to FIG. 37, the round sheath 1456 may be secured to the targeting guide 1448 and the plate 814 as well as to the femur 1402 in any suitable fashion, for example by a fastener connecting the targeting guide 1448 to the long bone 1402. For example, and as shown in FIG. 37, the round sheath 1456 is secured by a fastener, for example, an anchor bolt 1476. The anchor bolt 1476 may, for example, be slidably fitted within opening or hole 1478 of the round sheath 1456. The anchor bolt 1476 includes a threaded portion 1480 for engagement with hole 1472. Anchor bolt 1476 may include a cutting edge 1482 for removing bone in the femur 1402. It should be appreciate that the cutting edge 1482 may be used in conjunction with the hole 1472 or may in the alternative be utilized to prepare the entire opening in the femur 1402 eliminating the need to prepare the hole 1472 prior to the installation of the anchor bolt 1476.

The anchor bolt 1476 may be inserted in the direction of arrow 1484 into the hole 1478 of the round sheath 1456 and may engage hole 1472. The anchor bolt 1476 may be mounted by utilizing a power tool, for example power drill 1474.

While the anchor bolt 1476 serves a purpose to stabilize the targeting guide 1448 and to position the plate 814 with respect to the femur 1402, if the anchor bolt 1476 is rigidly secured to the targeting guide 1448 and is rigidly secured to the plate 814, percutaneous compression of the femur 1402 with the plate 814 may become difficult. For percutaneous compression of the fracture 1428 of the femur 1402, the second portion 1432 of the femur 1402 must be advanced in the direction of arrow 1486 toward the first portion 1430. To permit the motion in the direction of arrow 1486, the plate 814 must be permitted to move with respect to the anchor bolt 1476. It is thus desirable to permit the plate 814 to move in the direction of arrow 1486 with respect to the anchor bolt 1476 and similarly desirable to permit the targeting guide 1448 to move in the direction of arrow 1486 with respect to the anchor bolt 1476.

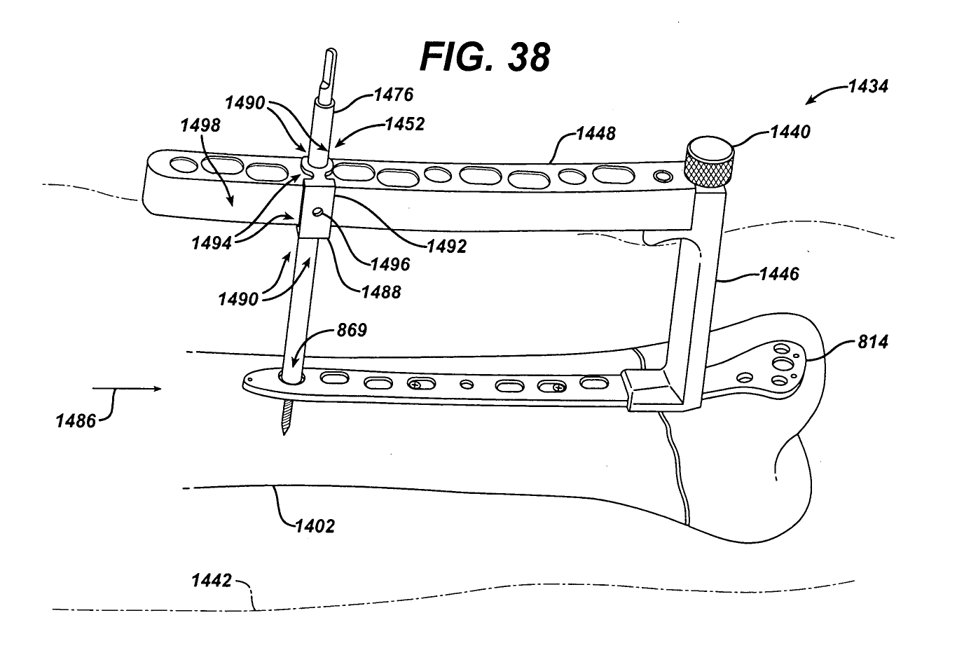

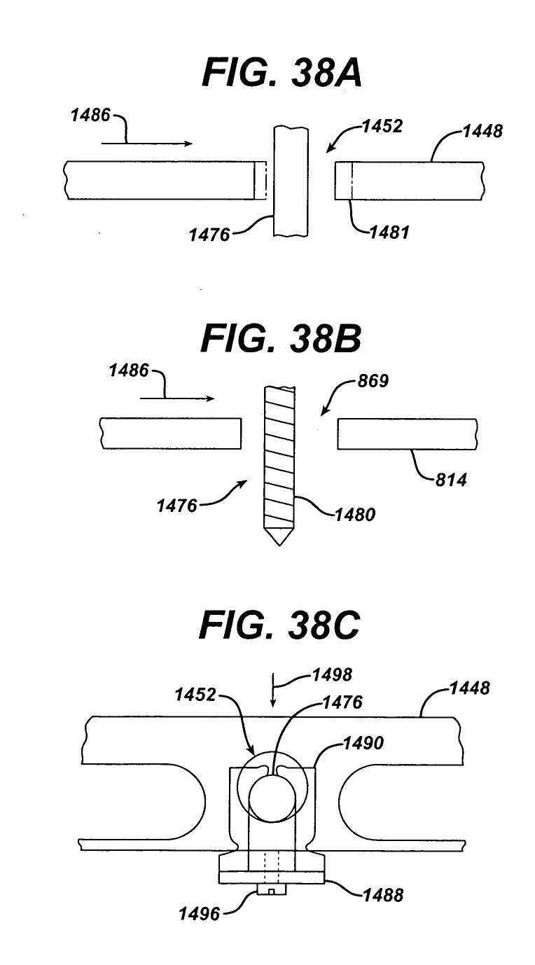

Referring now to FIG. 38, in order that the plate 814 may move in the direction of arrow 1486 with respect to the anchor bolt 1476, the round sheath 1456 (see FIG. 37) maybe removed from the targeting guide 1448 while the anchor bolt 1476 is still in position in the femur 1402. The removal of the round sheath 1456 from the anchor bolt 1476 permits relative motion between the anchor bolt 1476 and the targeting guide 1448 as well as between the anchor 1476 and the plate 814.

As shown in FIG. 38, the guide 1434 may further include an anchor clip 1448 to assist in stabilizing and centralizing the anchor bolt 1476 within the cylindrical opening 1452 of the targeting guide 1448. The anchor clip 1488 may have any configuration capable of centralizing and supporting the anchor bolt 1476 on the targeting guide 1448. For example, the anchor clip 1488 may include a pair of spaced apart resilient tines 1490 extending from a base 1492. An additional pair of tines 1490 may be positioned parallel to and spaced from the tines 1490. The tines 1490 may cooperate with grooves 1494 in the anchor bolt 1476. A screw 1496 located in the base 1492 of the anchor clip 1488 may serve to bias the anchor clip 1488 in the direction of arrow 1498.

Referring now to FIG. 38B, the threaded portion 1480 of the anchor bolt 1476 may be sized smaller in diameter than the threaded hole 869 of the plate 814 so that the plate 814 may be permitted to move in the direction of arrow 1486 with the respect to the anchor bolt 1476.

Referring now to FIG. 38A, the opening 1452 in the targeting guide 1448 is sized larger than the diameter of the anchor bolt 1476 so that the targeting guide 1448 may move in the direction of arrow 1486 with respect to the anchor bolt 1476 to position 1481 shown in phantom, thereby accommodating compression of the femur 1402.

Referring now to FIG. 38C, the anchor clip 1488 is shown in cooperation with the anchor bolt 1476. The anchor clip 1488 urges the anchor bolt 1476 in the direction of arrow 1498 urging the anchor bolt 1476 against the opening 1452 of the targeting guide 1448, thereby longitudinally centralizing the anchor bolt 1476 within the opening 1452 of the targeting guide 1448. It should be appreciated that since the tines 1490 of the anchor clip 1488 are resilient, the anchor bolt 1476 may be moveable within the opening 1452.

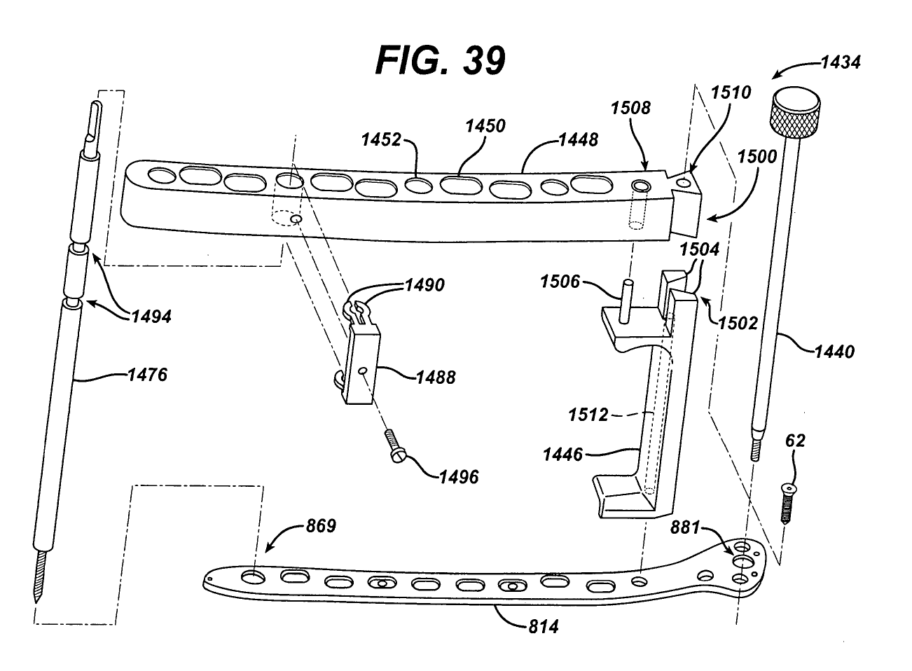

Referring now to FIG. 39, the guide 1434 is shown in an exploded view. The guide 1434, as shown in FIG. 39, is modular or made from a plurality of components. It should be appreciated that any one or a combination of the components of the guide 1434 may be combined into a solitary component. Similarly it should be appreciated that any of the components of the guide 1434, as shown in FIG. 39, may be modular or comprised of a plurality of components.

Each of the components of the guide 1434 may be made of any suitable durable material preferably of a material that is sterilizable by any known sterilization techniques, for example, auto claving. For example the guide 1434 may be made of components made of a metal, for example cobalt chromium alloy, a titanium alloy, or a stainless steel alloy. The targeting guide 1448 may be made of a radio-translucent material, for example a plastic. It should be appreciated that any of the components of the guide 1434 may be made of a plastic or a composition material.

To accommodate plates other than the femoral plate 814, it should be appreciated that the handle 1446 and in particular the targeting guide 1448, made be replaced with a targeting guide (not shown) which has a size and shape similar to that other plate. For example, the guide 1434 may be utilized with tibial plates, humeral plates, ulnar plates, or any other plate for use with the human skeleton. The guide 1434 may include other guides which may be substitute for the targeting guide 1448. The other guides (not shown) are designed to be compatible with the other forementioned plates.

Further, as shown in FIG. 39, the femoral plate 814 is for use with a left hand femoral plate 814. The targeting guide 1448 as shown in FIG. 39 is designed to be used with a left hand femoral plate. It should be appreciated that since the left hand femoral plate 814 and the right hand femoral plate (not shown) are mirror images of each other, the targeting guide 1448 may be rotated 180 degrees about its longitudinal axis and utilized for the opposite hand plate (the right hand plate).

The targeting guide 1448 may be fitted to the handle 1446 by, for example, a dove tailed connector 1500 located on an end of the targeting guide 1448 which is received in a slot 1502 formed between a pair of spaced apart protrusions 1504 in the upper portion of the handle 1446. A pin 1506 extending from the handle 1446 may engage in a hole 1508 formed in the targeting guide 1448. An opening 1510 in the doved tailed connector 1500 as well as an opening 1512 in the handle 1446 receives the connecting screw 1440.

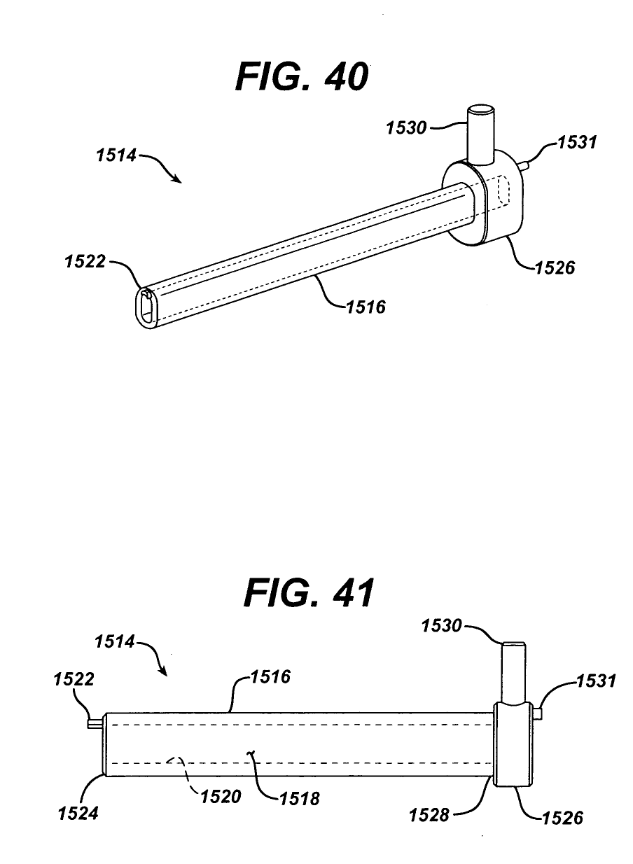

Referring now to FIG. 40 and 41, a sheath 1514 is shown. The sheath 1514 as shown is for cooperation with the elongated openings 1450 of the targeting guide 1448 (see FIG. 39). The sheath 1514 thus is in the form of an oval percutaneous sheath having an oval cross section to provide for a sliding fit of the percutaneous sheath 1514 into the elongated openings 1450 of the targeting guide 1448. The oval percutaneous sheath 1514 includes a hollow body 1516 having an oval outside diameter 1518 and an oval inside diameter 1520. The sheath 1514 includes a protrusion 1522 extending outwardly from first end 1524 of the body 1516. The sheath 1514 further includes a head 1526 extending outwardly from second end 1528 of the body 1516 of the sheath 1514. The head 1526 includes a visual assembly guide in the form of, for example, a pin 1530 extending transversely from the head 1526 and a orientation feature in the form of, for example, a pin 1531 extending from the head 1526 in the direction opposed to the body 1516.

The oval percutaneous sheath 1514 serves as a trocar or hollow tube through which the cortical screw 52 is inserted into the plate 814 to perform percutaneous compression.

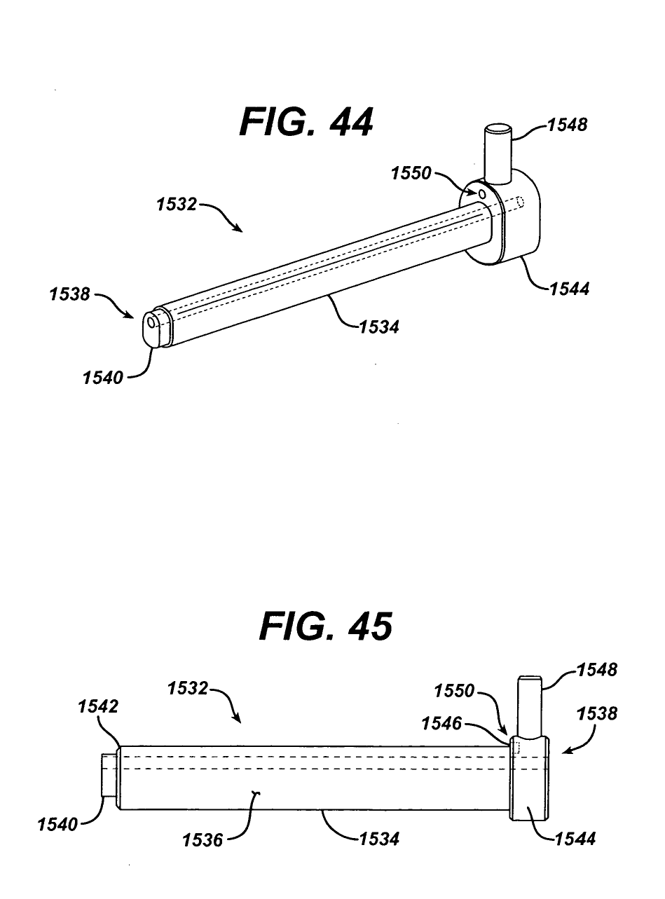

Referring now to FIGS. 44 and 45, a compressive drill guide 1532 is shown for use with the oval percutaneous sheath 1514 of FIGS. 40 and 41. Compression drill guide 1532 includes a body 1534 for slidably engagement within oval bone 1520 of the oval percutaneous sheath 1514 (see FIGS. 40 and 41). The body 1534 includes an oval periphery 1536 and a bore 1538. The bore 1538 is offset or off center to provide for compression as will be discussed later. A pilot 1540 extends outwardly from first end 1542 of the body 1534. The pilot 1540 is utilized for engagement with elongated openings 854 in the plate 814 (see FIG. 37).

The compression drill guide 1534 further includes a head 1544 extending outwardly from second end 1546 of the body 1534 of the guide 1532. A visual orientation guide 1548 may be utilized to orient the compression drill guide within the sheath 1514. The visual orientation guide 1548 may be in the form of a pin extending transversely from the head 1544. An axial orientation feature 1550 may be positioned in the head 1544 for cooperation with the orientation feature 1531 in the sheath 1514. The axial orientation feature 1550 may be in the form of, for example, a recessed cylindrical opening.

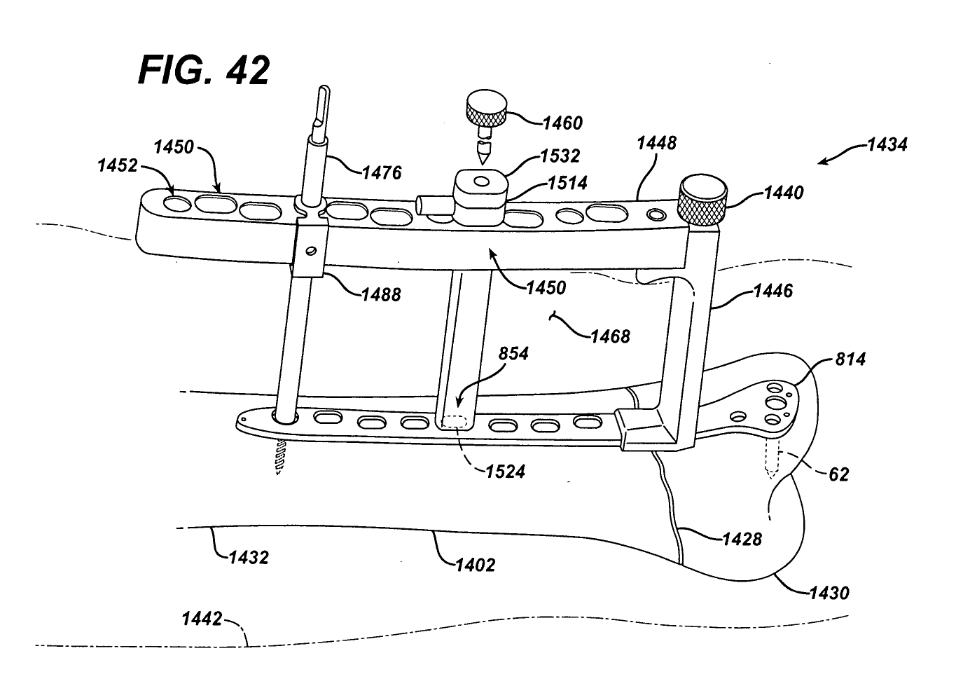

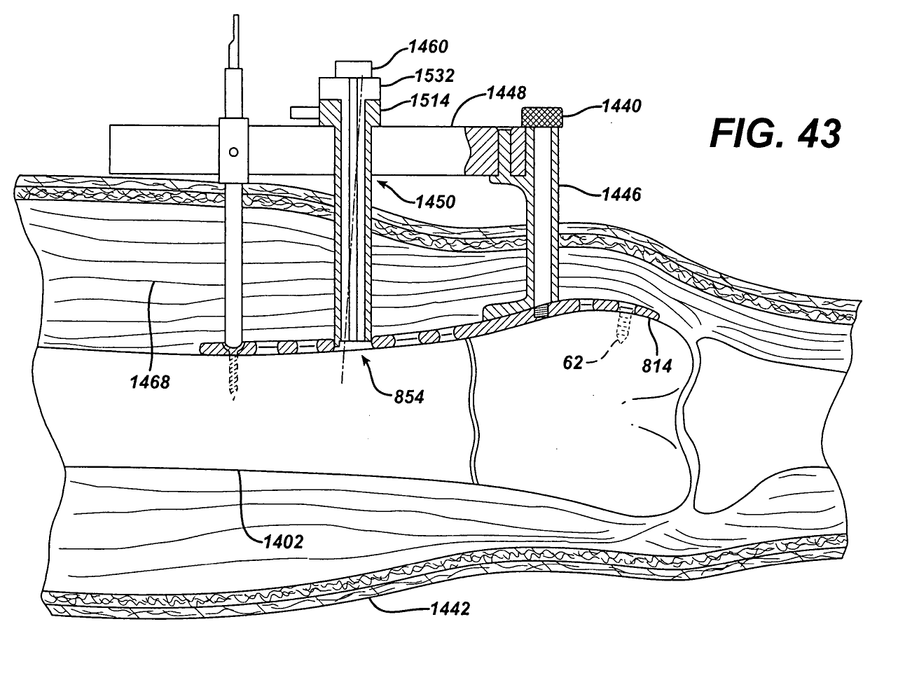

Referring now to FIGS. 42 and 43, the oval percutaneous sheath 1514, the drill guide 1532 and the pin 1416 are shown assembled to each other and in position in the guide 1434. The sheath 1514, the drill guide 1532 and the pin 1460 are preassembled into each other with the drill guide 1534 being positioned in the sheath 1514 and the pin 1416 being positioned in the drill guide 1532. The oval precutaneous sheath 1514 is then positioned over one of the elongated openings 1450 that is in alignment with one of the elongated openings 854 of the plate 814. A small incision is made through the skin of the thigh 1442 immediately under the appropriate elongated opening 1450 and the sheath 1415 together with the guide 1532 and pin 1460 are passed through the elongated opening 1450, through the incision and passed percutaneously through the soft tissue 1468 until the first end 1524 of the sheath 1415 rests against the plate 814. After the sheath 1514 is positioned against the plate 814, the pin 1460 is removed.

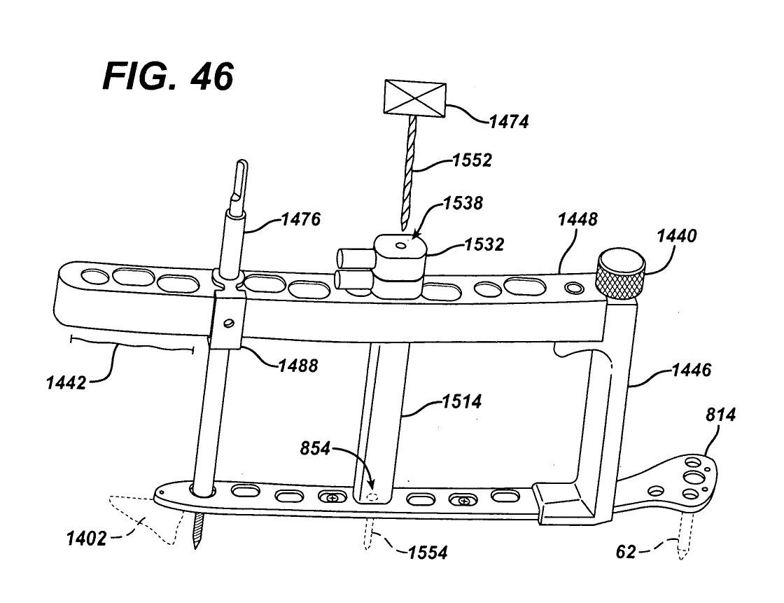

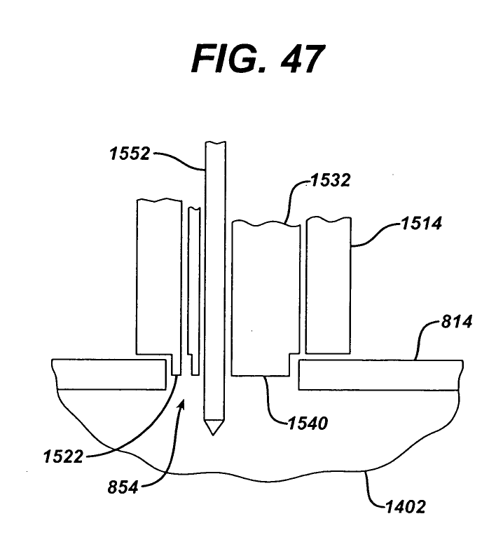

Referring now to FIGS. 46 and 47, the proper positioning of the percutaneous sheath 1514 against the plate 814 is shown in greater detail. Thus shown in FIG. 47, the pilot 1540 of the drill guide 1532 is positioned in the opening 854 as is the protrusion 1522 of the sheath 1514. The pilot 1540 and the protrusion 1522 serve to position the drill guide 1532 and the sheath 1514 with respect to the plate 814.

Referring now to FIGS. 46 and 47, after the sheath 1514 is positioned on plate 814, a tool in the form of a drill 1552 is inserted in the bore 1538 of the drill guide 1532 and is utilized to form drilled hole 1554 in the bone 1402. A power tool 1474 maybe utilized to rotate the drill 1552.

After the drilled hole 1554 is formed in the bone 1402, the drill guide 1532 is removed from the oval percutaneous sheath 1514.

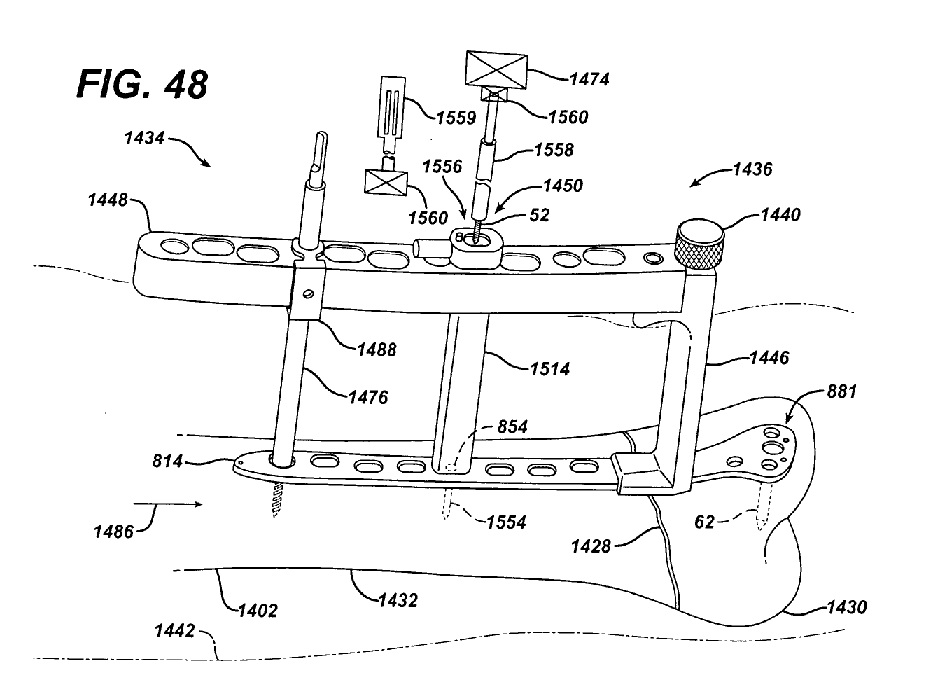

Referring now to FIGS. 48, 49, and 50, the guide 1434 is shown for inserting the percutaneous compression screw in the form of, for example, cortical screw 52. It should be appreciated that the percutaneous compression screw may be in the form of any screw capable of performing the percutaneous compression. While the screw 52 as shown in FIG. 48, is a cortical screw, it should be appreciated that particularly for thin shelled cortical bone, a cancellous screw (not shown) may be more suitable to provide for the compression of the bone. For example, a cancellous screw such as screw 980 (see FIG. 21) may be used.

The screw 52 is inserted into internal opening 1550 of the sheath 1514 by, for example, a screwdriver 1558. The screwdriver 1558 may be manually operated by handle 1559 and may include a torque limiter 1560 to limit the torque applied to this screwdriver 1558. It should be appreciated that power tool 1474 may be connected to the screwdriver 1558 for driving the screwdriver 1558.

The screwdriver 1558 and the cortical screw 52 are inserted into the internal opening 1556 until the screw 52 engages in the predrilled hole 1554 in the femur 1402. It should be appreciated that the invention may be practiced with the predrilled hole 1554 omitted. In such case, the screw 52 includes a self-drilling and tapping feature such that the screw 52 may directly engage the femur 1402.

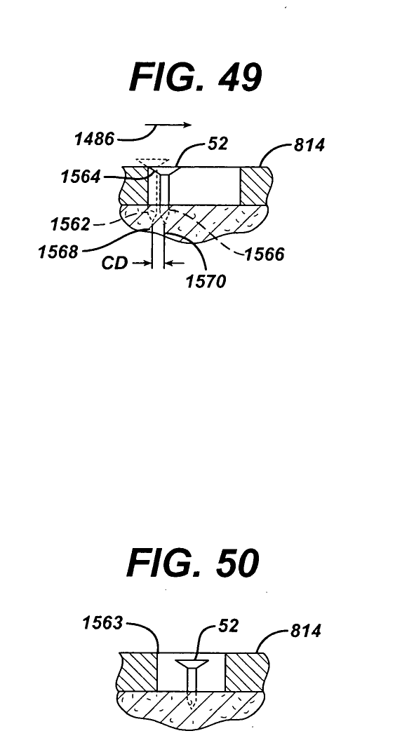

Referring now to FIG. 49, the screw 52 is shown in a first position 1562 as shown in phantom. In the first position 1562 the screw is shown in initial engagement with the plate 814. The screw 52 preferably includes an angled contact surface 1564 which engages internal edge 1563 of the plate 814. As the screw 52 continues to be engaged into the femur 1402, the screw 52 is moved in the direction of arrow 1486 into a second position 1566 shown in solid. First position 1562 defines a first position centerline 1568 while the second position 1566 defines a second position centerline 1570. The centerlines 1568 and 1570 define a distance CD there between. The distance CD defines a compression distance for the compression. A typically dimension of CD may be from, for example, one half to one and half millimeters. It should be appreciated that the invention may be practiced with a dimension CD less than half a millimeter or larger than a millimeter and a half. It should be appreciated that the dimension CD is limited by the angled contact surface 1564 of the screw 1552 and by the size of the elongated opening 854 of the plate 814.

It should be appreciated that after the screw 52 is fully engaged against the femur 1402, additional elongated openings 854 may be used with additional screws 52 in any similar fashion to provide additional compression to the femur 1402. Referring now to FIG. 50, if a second compression is to occur, the screw 52 is preferably positioned spaced from the internal edge 1563 of the plate 814.

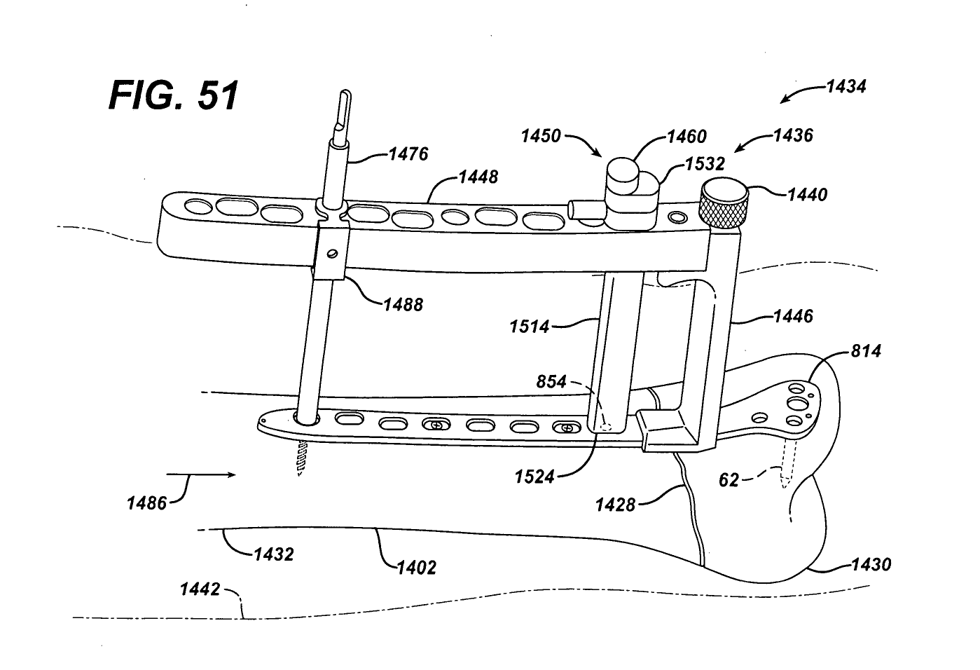

Referring now to FIG. 51, sheath 1514 of the guide 1434 is shown in position on the targeting guide 1448 in alignment with a different elongated opening 854 of plate 814 and a different elongated opening 1450 of the targeting guide 1448 than that of FIG. 48. By providing the guide 1434 with the sheath 1514 in the position as shown in FIG. 51, an additional or second percutanceous compression can be performed on the femur 1402.

It should be appreciated in order to perform a second compression on the femur 1402 to accommodate further compression at the fracture 1428 between the first portion 1430 of the femur 1402 and the second portion 1432 of the femur 1402, both the position of the sheath 1514 with respect to the first elongated opening 854 and in its second position against the second elongated opening 854 must both be on the same, for example, second portion 1432 of the femur 1402.

In order to perform the second compression, the sheath 1514, the drill guide 1532 and the pin 1460 maybe preassembled together. A small incision is made in thigh 1442 under the elongated opening 1450 permitting the sheath 1514, the drill guide 1532 and pin 1460 to be advanced through the elongated opening 1450 and into the soft tissue 1468 until first end 1524 of the sheath 1514 seats against the plate 814. After the sheath 1514 is fully seated against the plate 814, the pin 1460 is removed from the drill guide 1532.

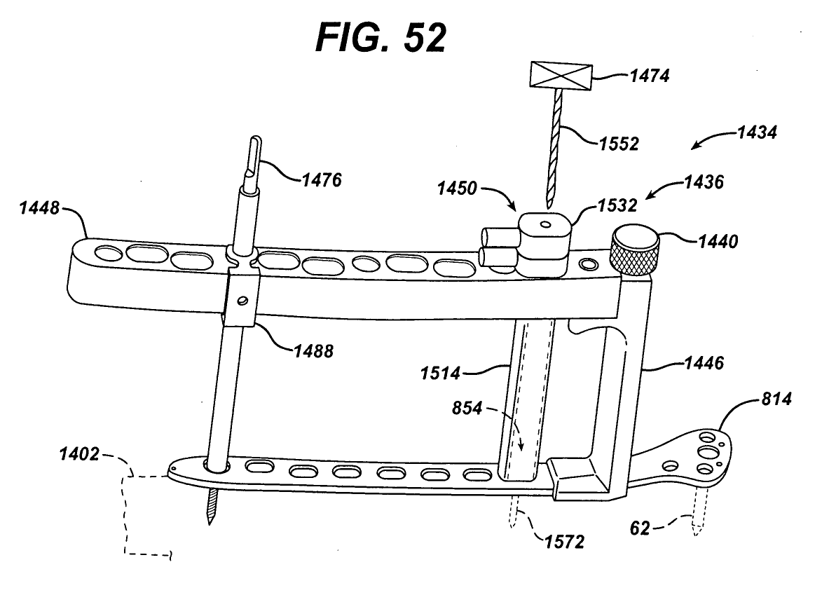

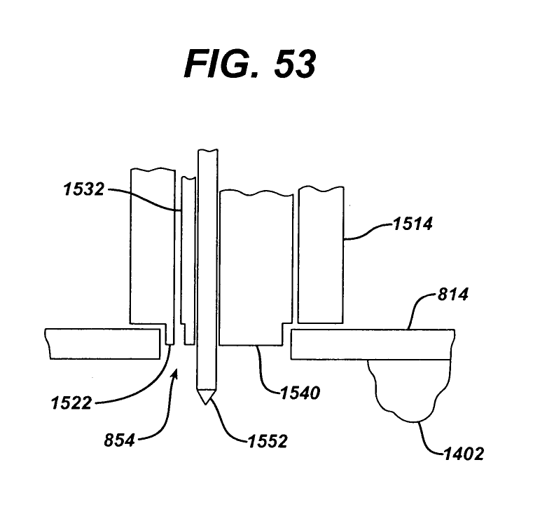

Referring now to FIGS. 52 and 53, the drill guide 1434 is shown utilized in repairing a second predrilled opening 1572 in the long bone 1402 for providing the second compression. The drill 1552 is inserted into the drill guide 1532 until it engages in the femur 1402 to provide the predrilled opening 1572.

Referring now to FIG. 52, the sheath 1514 is shown in position in the elongated opening 854 of the plate 814. The protrusion 1522 of the sheath 1514 and the pilot 1540 of the drill guide 1532 serve to position the drill 1552 in the proper position in the bone 1402 with respect to the plate 814 to accommodate the percutaneous compression.

After the predrilled opening 1572 is formed in the long bone 1402, the drill 1552 powered by, for example, the power drill 1474 is removed from the drill guide 1532. After the drill 1552 has been removed, the drill guide 1532 may be removed from the oval percutaneous sheath 1514.

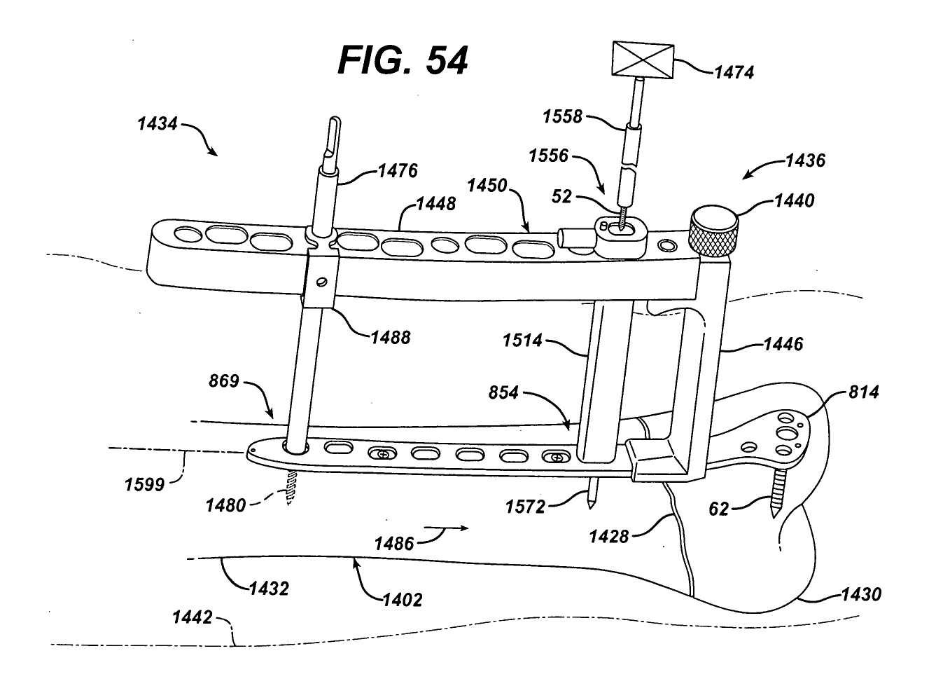

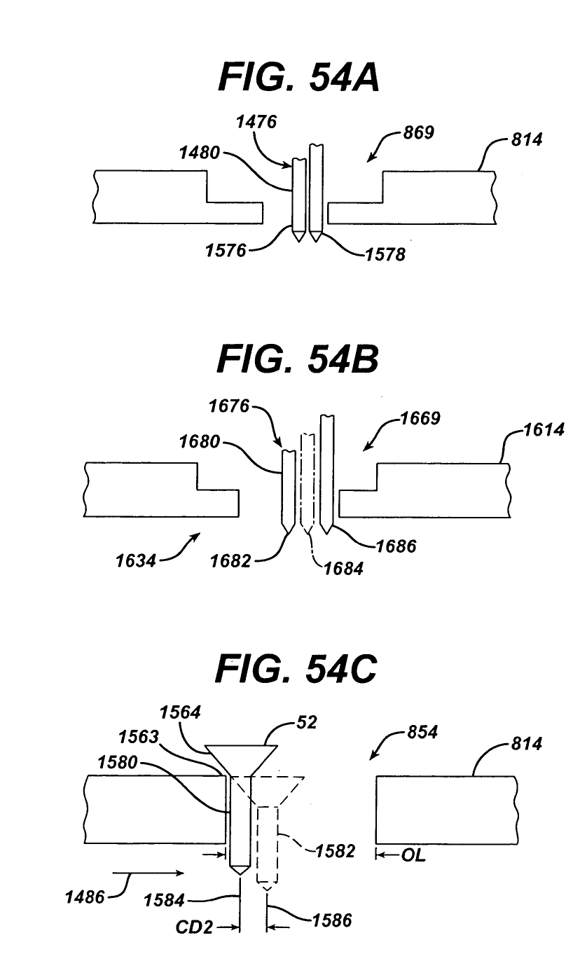

Referring now to FIGS. 54, 54A, 54B, and FIG. 55, the guide 1432 is shown for use in performing the second percutaneous compression of the long bone 1402. It should be appreciated that in order for the compression of the femur 1402 to occur, the second portion 1432 of the femur 1402 must move in the direction of arrow 1486 along longitudinal axis 1599 toward the first portion 1430 of the femur 1402. The location of threaded portion 1480 of the anchor bolt 1476 in the threaded hole 869 and the location of any of the cortical screw 52 in any of the elongated openings 854 of the plate 814 may be such that the second portion 1432 of the femur 1402, may be prohibited from advancing in the direction of arrow 1486, thus limiting the effect of a second or subsequent percanteous compression. Thus in order that the second compression be effective, the positioning of the anchor bolt 1476 and the cortical screws 52 on the first or earlier compressions must allow for further motion of the second portion 1432 of the femur 1402 in the direction of arrow 1486.

Referring now to FIG. 54A, anchor bolt 1476 is shown in position in threaded hole 869 of the plate 814. First position 1576 of the threaded portion 1480 of the anchor bolt 1476 is shown in solid. Note that the first position 1576 of the threaded portion 1480 is spaced from the walls of the threaded hole 869 to permit additional compression to be done on a subsequent percutaneous compression of the bone 1402. A second position 1578 of the threaded portion 1480 of the anchor bolt 1476 is shown, in phantom. The second position 1578 is shown adjacent of the edge of, or side wall of, the threaded hole 869 and indicates that the second compression may, with this design, be the last effective compression.

It should be appreciated that the anchor bolts or threaded holes in the plate may be designed such that additional compressions may be possible in addition to a first and second compression. For example, as shown in FIG. 54B, another embodiment of the present invention is shown as guide 1634. Guide 1634 includes plate 1614 having a threaded hole 1669 which is substantially larger than the threaded hole 869 of the plate 814 of FIG. 54A. As shown as in FIG. 54B, the threaded portion 1680 of the anchor bolt 1676 may be positioned in a first position 1682 shown in solid as well as a second position 1684 as shown in phantom. The guide 1634 of FIG. 54B also allows for a third compression position 1686 as shown in phantom.

It should be appreciated that alternatively that anchor bolt 1476 may be removed from the guide 1434 when performing a second or subsequent compression, thus eliminating the issue regarding the limitation of compression caused by the positioning of the threaded portion 1480 of the anchor bolt 1476 in the threaded hole 869.

Referring again to FIG. 54, the screw 52 is shown inserted in opening 1556 of the sheath 1514. The screw 52 may be driven by, for example, screwdriver 1558 driven by, for example, power drill 1474. The screw 52 is inserted in the opening 1556 until it engages in predrilled opening 1572 in the long bone 1402. It should be appreciated that the screw 52 may engage the femur 1402 with the absence of the predrilled opening 1572. In such case the screw 52 may include a self-drilling and self-tapping feature.

Referring now to FIG. 55, the screw 52 is shown in a first position 1580 as shown in solid in which the angled surface 1564 of the screw 52 is in initial contact with internal edge 1563 of the plate 814. As the screw 52 is continued to be engaged into the bone 1402, the screw 52 moves in the direction of arrow 1486 to second position 1582 as shown in phantom. The screw 52 thus moves from a first centerline 1584 to a second centerline 1586.

The distance between the centerlines 1584 and 1586 is defined by, for example, a dimension CD2. The dimension CD2 defines the movement of the second portion 1432 of the bone 1402 and thus the compression of the fracture 1428 accomplished by the second compression. The dimension CD2 may be, for example, one half to one and a half millimeters.

It should be appreciated that the dimension CD2 may be greater or lesser than one half to one and a half millimeters. The internal elongated opening 854 in the plate 814 defines an opening length OL. It should be appreciated that the opening length OL and the compression dimension CD2 may limit the number of subsequent compressions available. It should be appreciated that as each elongated opening 854 of the plate 814 is utilized with a compression screw 52, the number of subsequent compressions available on the bone are limited by the number of elongated openings 854 which do not yet have a screw 52 associated with them. As shown in FIG. 54, if the number of elongated openings is as shown total of five, then five consecutive compressions may be available depending on the dimension OL as compared to the compression accomplished in each compression or dimension CD2 as well as the shank diameter of the screw 52.

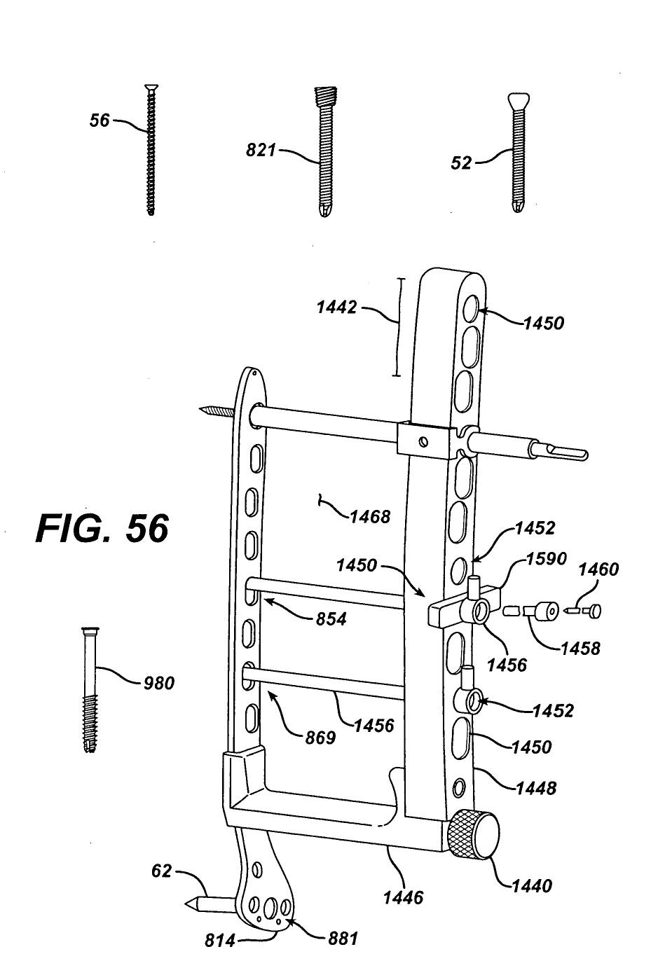

Referring now to FIGS. 56 through 62, the guide 1434 of the present invention may be used to provide percutaneous attachment of the plate 814 without compression. For example, and referring now to FIG. 56, the guide 1434 may be utilized to percutaneously install screws in the form of, for example, cortical screw 52 or cortical screw 821 as well as to install cancellous screw, for example, cancellous screws 56 and 980. The screws 52, 56, 821, and 980 may be installed percutaneously without compression. Both the elongated openings 854 and the threaded openings 869 of the plate 814 may be utilized to provide percutaneous noncompression securement of the plate 814.

When utilizing the guide 1434 to provide percutaneous installation of a screw into the threaded openings 869 of the plate 814, the trocar pin 1460 may be is assembled into the bushing 1458 which is assembled into the round sheath 1456. A small incision is made in thigh 1442 in alignment with the appropriate round opening 1452 which is in alignment with the threaded opening 869 of the plate 814 in which the screw is to be inserted. The round sheath 1456, bushing 1458 and pin 1460 are then inserted through the round opening 1452 and into position until the round sheath 1456 engages the plate 814.

Referring now to FIGS. 58A and 56, the sheath 1456 is shown in greater detail in engagement with the plate 814. The sheath 1456 seats against elongated recess 867 of the plate 814. The pin 1460 is then removed from the bushing 1458 and drill 1552 is inserted in the bushing 1458 to form drill opening 1588 in the bone 1402. The bushing 1458 is then removed from the round sheath 1456 and a screw, for example, cortical screw 56 or cancellous screw 980 is inserted into the opening of the round sheath 1456 and installed in opening 1588. The locking cortical screw 821 may alternatively be positioned through the round sheath 1456 and engaged into opening 1588. The locked screw 821 is rigidly secured to the plate 814.

Percutaneous installation of noncompression screws into the plate 814 may also be accomplished with the use of the guide 1434. For example and as shown in FIG. 56, an elongated opening 854 may be chosen to receive a screw, for example, screw 56. An elongated opening 1450 is then selected in alignment with the elongated opening 854 of the plate 814. A small incision is then made in the skin of the thigh 1442 under the elongated opening 1450. A plug 1590 is then inserted in the elongated opening 1450. The assembly of the round sheath 1456, bushing 1458, and trocar pin 1460 are then inserted through the plug 1590 and into the soft tissue 1468 until the round sheath 1456 contacts the plate 814.

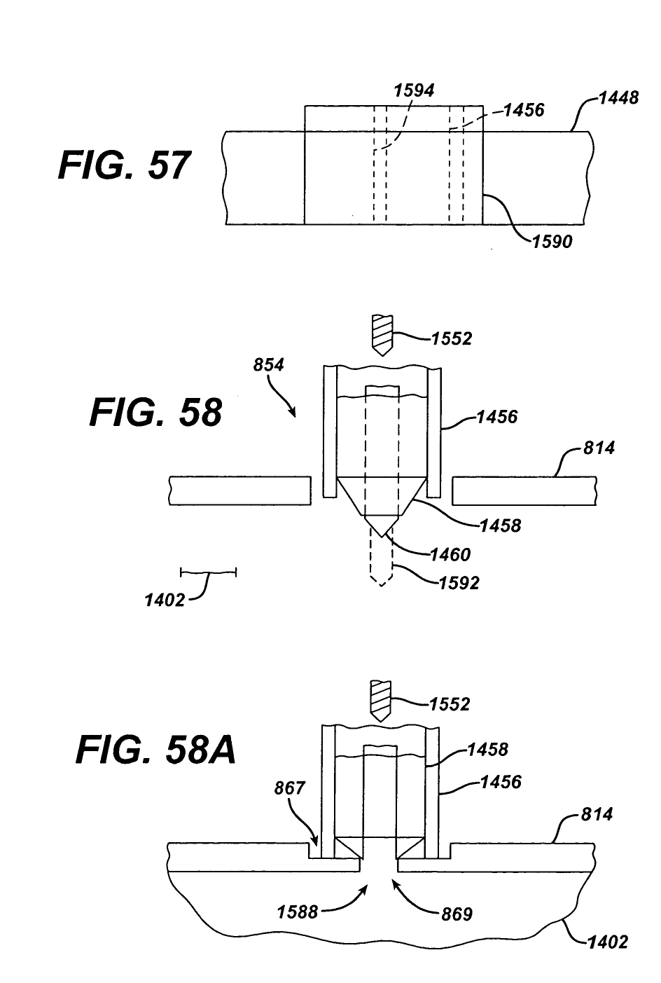

Referring now to FIG. 57, the round sheath 1456 is shown in greater detail in position in the plate 814. The plug 1590 includes a bore 1594 into which the sheath 1486 is slidably received. As shown in FIG. 57 the bore 1594 is offset from centerline in order that the screw 56 is properly positioned with respect to the elongated opening 854 of the plate 814 (see FIG. 56).

Referring now to FIG. 58, the pin 1460 is then removed from the bushing 1458 and a drill 1552 is inserted in the bushing 1458 and rotated to prepare pre-drilled opening 1592 in the bone 1402. The bushing 1458 is then removed and the screw, for example, cortical screw 56, is inserted through the round sheath 1456 and threaded into opening 1592.

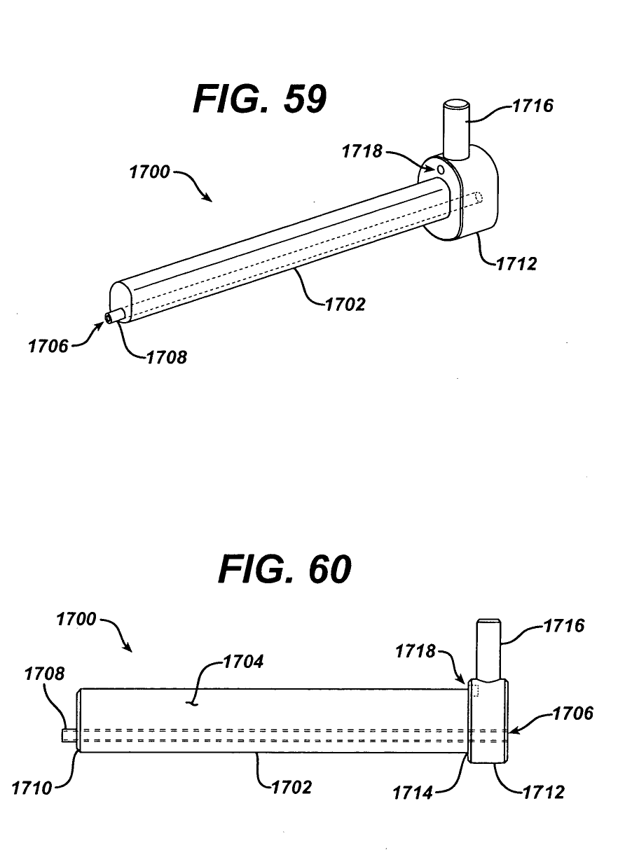

Alternatively, the noncompression installation of screws utilizing the guide 1434 may be accomplished in connection with the oval percutaneous sheath 1514 of FIG. 54. For example, the guide 1434 may further include a noncompression oval drill guide 1700, shown in FIGS. 59 and 60. The drill guide 1700 fits within the sheath 1514.

As shown in FIG. 59 and 60, the drill guide 1700 includes a body 1702 having an oval outside diameter 1704. The body 1702 further includes a cylindrical bore 1706 compatible with the pin 1460 as well as the drill 1552. The guide 1700 further includes a pilot 1708 extending from first end 1710 of the body 1702. The guide 1700 further includes a head 1712 extending from second end 1714 of the body 1702. A visualization guide 1716 may extend transversely from the head 1712. An opening 1718 may be positioned in the head 1712 adjacent to body 1702.

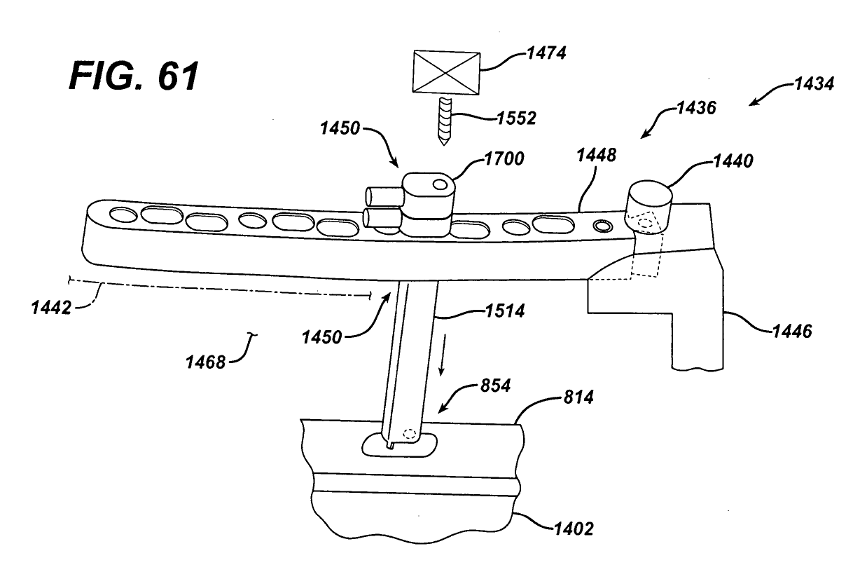

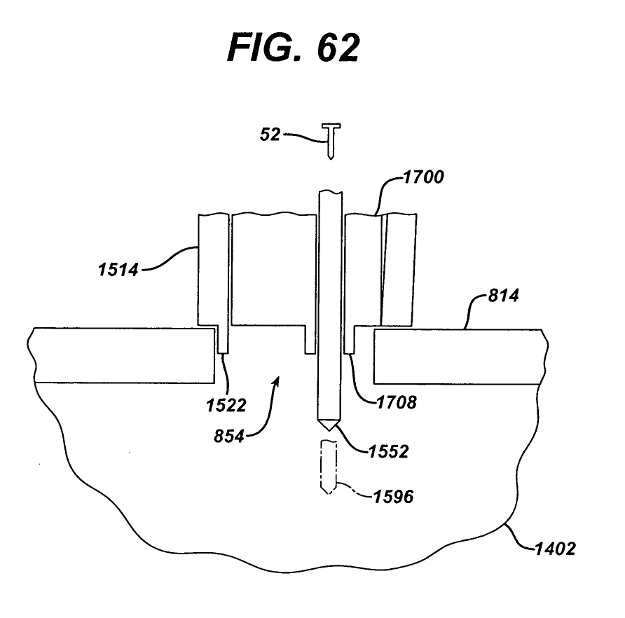

Referring now to FIGS. 61 and 62, the guide 1434 is shown with the noncompression oval drill guide 1700 being utilized to perform a noncompression percutaneous installation of a screw, for example, screw 52, into the plate 814 in the elongated opening 854 in the plate 814. An elongated opening 854 is chosen for receiving the screw 52. The elongated opening 1450 of the targeting guide 1448 in alignment with the chosen elongated opening 854 is selected. A small incision is made in the thigh 1442 and the sheath 1514, the guide 1700 and the pin 1460 having been preassembled, are inserted through the elongated opening 1450 and into the soft tissue 1468 until the sheath 1514 is engaged in the plate 814.

Referring now to FIG. 62, the protrusion 1522 of the sheath 1514 and the pilot 1708 of the drill guide 1700 provide alignment of the sheath assembly to the opening 854 of the plate 814. The pin 1460 is then removed from the drill guide 1700 and drill 1552 is inserted into the guide 1700 to prepare drilled hole 1596 in the bone 1402. The drill guide 1700 is then removed from the sheath 1514 and the screw 52 is inserted through the sheath 1514 and engaged in drilled hole 1596.

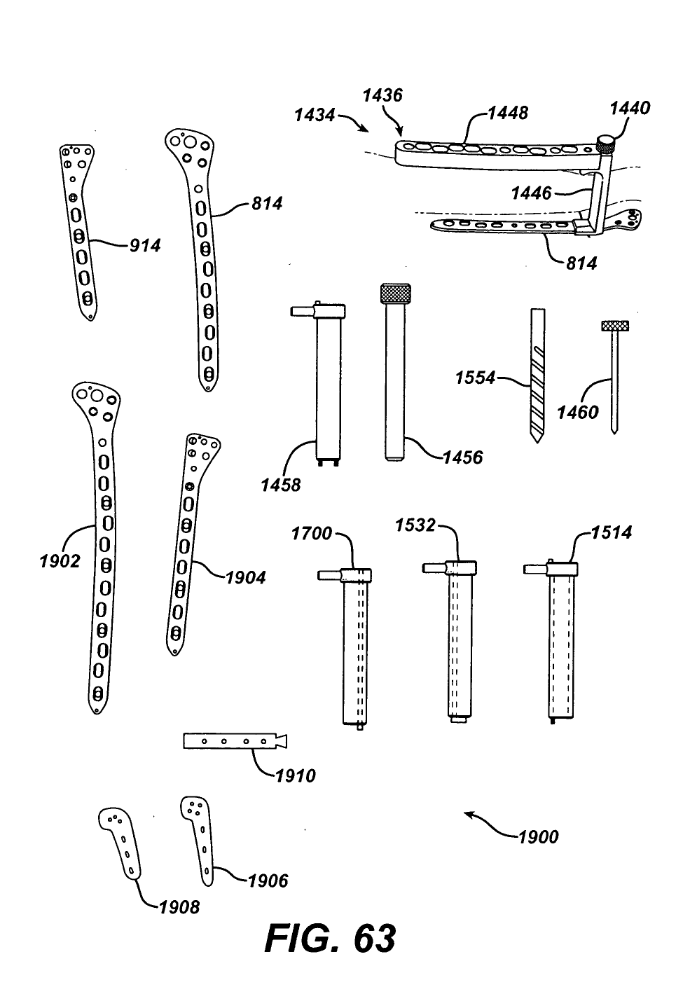

Referring now to FIG. 63, a kit 1900 is shown for providing percutaneous compression of a bone plate. The kit 1900 includes, for example, the guide 1434 having, for example, a targeting guide 1434 including, for example, a body 1436 for attachment to, for example, a plate 814. The body 1436 may include a targeting guide 1448 secured to, for example, handle 1446 by, for example, a connecting screw 1440. Kit 1900 may further include a series of drills, pins, bushings, and sheaths to accomplish the percutaneous installation of screws. For example, the kit 1900 may include an oval sheath 1514 as well as a round sheath 1456. The kit 1900 may include a compressive drill bushing 1532 and noncompression drill bushings 1700 and 1458.

The kit 1900 may also include a drill 1554 for preparing the bone to receive a screw. The kit 1900 may also include a pin 1460 for installing the sheaths and bushings percutaneously. The kit 1900 may further include a single plate, for example, a femoral plate 814 or may also include, in the alternative or in addition, tibial plate 914. Kit 1900 may further include a second or different femoral plate 1902 as well as a second or different tibial plate 1904. It should be appreciated that the kit 1900 may further include plates for other bones of the body. For example, the kit 1900 may include a humeral plate 1906 or an ulnar plate 1908. The targeting guide 1900 may further include a second targeting guide 1910 for use with, for example, a different plate, for example, the tibial plate 910 or any of the femoral plate 1902, tibial plate 1904, humeral plate 1906, or ulnar plate 1908.

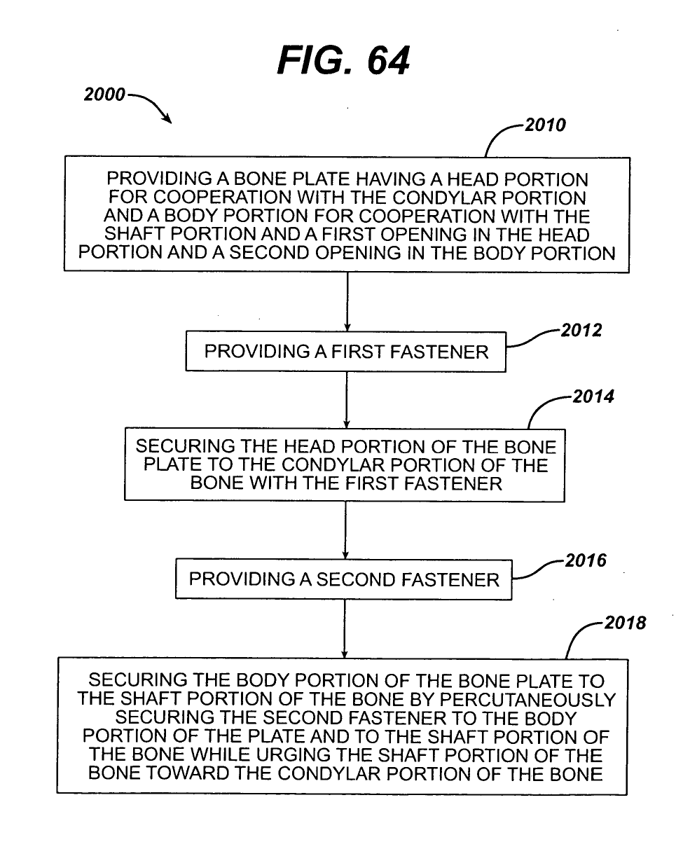

Referring now to FIG. 64, another embodiment of the present invention is shown as method 2000. Method 2000 includes a first step 2010 of providing a bone plate having a head portion for cooperation with the condylar portion and a body portion for cooperation with the shaft portion and a first opening in the head portion and a second opening in the body portion. The method 2000 further includes a second step 2012 of providing a first fastener and a third step 2014 of securing the head portion of the bone plate to the condylar portion of the bone with the first fastener. The method 2000 further includes a fourth step 2016 of providing a second fastener and fifth step 2018 of securing the body portion of the bone plate to the shaft portion of the bone by percutaneously securing the second fastener to the body portion of the plate and to the shaft portion of the bone while urging the shaft portion of the bone toward the condylar portion of the bone.

By providing a fracture repair system including a bushing to permit polyaxial rotation of the bushing within the hole plate an attachment component may be secured to a plate with the ability to position divergently to secure the fracture of the bone most efficiently. For example bone fragments may be reached by orienting the attachment component relative to the plate in such a direction to reach various bone fragments.

By providing a fracture repair system including a bushing with a spherical outside diameter in cooperation with a plate having a spherical bore, a low-friction polyaxial rotation of the attachment component relative to the plate is possible.

By providing a fracture repair system including a bushing having a tapered threaded bore in cooperation with a tapered threaded or non-threaded attachment component, the attachment component may be rigidly secured in a variety of orientations.

By providing a fracture repair system including a polyaxial bushing which may be rigidly secured to a plate and including a closely conforming plate which closely conforms to the condyle areas of a long bone the fragments fractured components within the condyle areas may be effectively and efficiently contained.

By providing a fracture repair system including a threaded alignment hole for securing a jig for drilling and threading the plate to the bone perpendicularly, a simple to use effective efficient bone plate system can be provided.

By providing a bone plate including a contoured tip for percutaneous insertion, a bone plate may be provided percutaneously for minimally invasive surgery. Such a contoured tip permits easy and effective insertion and alignment of the plate to the bone.

Providing a fracture repair system that provides for home plate compression percutaneously, large fragments of bone may be pulled or aligned together encouraging faster healing of the bone site. The percutaneous installation provides for a small scar and reduced infection as well as shorter healing periods.

By providing a fracture repair system including a plurality of components which may be combined in a plurality of combinations, large variety of bone plates may be used to provide percutaneous compression with a minimum amount of inventory.

Although the invention has been described in detail with reference to a preferred embodiment, variations and modifications exist within the scope and spirit of the invention as described and defined in the following claims.

1. A system for percutaneous fracture repair of a bone, the system comprising:
a plate having a first feature and a second feature, the first feature and the second feature being spaced apart from each other, said plate defining a longitudinal axis thereof;
a first attachment component operably associated with the first feature, said first attachment component adapted for cooperation with the bone; and
a second attachment component operably associated with the second feature, said second attachment component being percutaneously inserted into the second feature, said second attachment component operably associated with the plate to provide a compressive force in the bone, the compressive force having a component thereof in the longitudinal axis, said second attachment component adapted for cooperation with the bone.

2. The system as in claim 1, wherein at least one of the first feature and the second feature is defined by an opening through said plate.

3. The system as in claim 1, wherein at least one of said first attachment component and said second attachment component comprises a screw.

4. The system as in claim 1:
wherein said second attachment component defines a first surface thereof; and
wherein said second feature defines a first surface thereof, the first surface of said second attachment component cooperating with the first surface of said second feature as said second attachment component advances toward said second feature to compress the bone.

5. The system as in claim 1, wherein the bone defines a fracture thereof, the fracture defining a first bone portion and a second bone portion, said first attachment component and said first feature associated with the first bone portion and said second attachment component and said second feature associated with the second bone portion.

6. The system as in claim 1, further comprising a guide for guiding said second attachment component into engagement with said second feature and into said bone.

7. The system as in claim 6, wherein said guide comprises:
a body attachable to said plate; and
a tube extending from said body for guiding said second attachment component into cooperation with the second feature.

8. The system as in claim 7, further comprising:
a drill;
a first bushing slidably fitted to said tube, said first bushing adapted to guide said drill into a first relationship with said second feature and into the bone, said drill hereby forming a first bone hole in the bone, whereby when said second attachment component is passed in the tube and engaged into the first bone hole, said plate cooperates with said second attachment component so that the bone is under compression; and
a second bushing slidably fitted to said tube, said second bushing adapted to guide said drill into a second relationship with said second feature and into the bone, said drill hereby forming a second bone hole in the bone, whereby when said second attachment component is passed in the tube and engaged into the first bone hole, said plate cooperates with said second attachment component so that the bone is not under compression.

9. The system as in claim 6, wherein said guide comprises a portion of which is radiolucent.

10. The system as in claim 1, wherein said plate includes a portion thereof shaped to closely conform to the bone.

11. A system for percutaneous fracture repair of a long bone including a shaft portion and a condylar portion thereof, the long bone defining a fracture thereof, the fracture positioned at least partially between the shaft portion and the condylar portion, the system comprising:
a plate including a first portion for cooperation with the condylar portion and a second portion for cooperation with the shaft portion, said first portion defining a first opening there through and said second portion defining a second opening there through, the first opening and the second opening being spaced apart from each other, said plate defining a longitudinal axis thereof extending from the first portion to the second portion of said plate;
a first fastener adapted to at least partially pass through the first opening, said first fastener adapted to at least partially engage with the condylar portion of the bone; and
a second fastener adapted to at least partially pass through the second opening, said second fastener adapted to at least partially engage with the shaft portion of the bone, said second fastener being percutaneously inserted into the second opening, said second fastener contacting said plate adjacent with the second opening of said plate to provide a compressive force in the bone, the compressive force having a component thereof in the longitudinal axis operably associated with the bone to provide a compressive force in the bone.

12. The system as in claim 11, wherein at least one of the first opening and the second opening is defined by an oval opening through said plate.

13. The system as in claim 11, wherein at least one of said first fastener and said second fastener comprises one of a screw and a pin.

14. The system as in claim 11:
wherein said second fastener defines a first surface thereof; and
wherein the second portion of said plate adjacent the second opening defines a first surface thereof, the first surface of said second fastener cooperating with the first surface of the second portion of said plate as said second fastener advances toward said second hole to advance the shaft portion of the bone toward the condylar portion of the bone to serve to compress the bone.

15. The system as in claim 11, further comprising a guide for guiding said second fastener at least partially through the second hole and into said bone.

16. The system as in claim 15, wherein said guide comprises:
a body attachable to said plate; and
a tube extending from said body for guiding said second fastener at least partially through the second hole.

17. The system as in claim 16, further comprising:
a drill;
a first bushing slidably fitted to said tube, said first bushing adapted to guide at least a portion of said drill through the second plate hole and into the bone, said drill hereby forming a first bone hole in the bone, whereby when said second fastener is passed in the tube and engaged into the first bone hole in a first relationship whereby a surface of said plate adjacent the second plate hole cooperates with said second fastener so that the shaft portion of the bone is advanced toward the condylar portion of the bone to compress the long bone; and
a second bushing slidably fitted to said tube, said second bushing adapted to guide said drill into a second relationship with said second plate hole and into the bone, said drill hereby forming a second bone hole in the bone, whereby when said second fastener is passed in the tube and engaged into the second bone hole in a second relationship whereby said plate cooperates with said second fastener so that the shaft portion of the bone is remains in its previous relationship with the condylar portion of the bone to assure that the long bone not under compression.

18. The system as in claim 15, wherein said guide comprises a portion of which is radiolucent.

19. A guide to assist in the percutaneous fracture repair of a bone having a first bone location and a spaced apart second bone location, the guide to be used to guide a fastener at least partially through an opening in a bone plate and into the bone, said guide comprising:
a body attachable to the bone plate adjacent the first bone location; and
a tube fitted to said body for guiding the fastener percutaneously at least partially through the bone plate opening in the bone plate and into the bone at the second bone location, said body and said tube adapted to cooperate with the bone plate and with the fastener so that the bone is under compression between the first bone location and the second bone location.

20. The guide as in claim 19, further comprising:
a drill;
a first bushing slidably fitted to said tube, said first bushing adapted to guide said drill into a first relationship with the bone plate adjacent the bone plate opening and into the bone, said drill hereby forming a first opening in the bone, whereby when the fastener is passed in the tube and engaged into the first bone hole, the bone plate cooperates with the fastener so that the bone is under compression between the first bone location and the second bone location; and
a second bushing slidably fitted to said tube, said second bushing adapted to guide said drill into a second relationship the bone plate adjacent the bone plate opening and into the bone, said drill hereby forming a second bone hole in the bone, whereby when the fastener is passed in the tube and engaged into the first bone hole, the bone plate cooperates with said second attachment component so that the bone is not under compression between the first bone location and the second bone location.

21. The system as in claim 19, wherein said guide comprises a portion of which is radiolucent.

22. The guide as in claim 19, wherein said tube includes a protrusion for cooperation with the bone plate adjacent the bone plate opening.

23. The guide as in claim 21, at least one of said first bushing and said second bushing includes a bushing protrusion, the bushing protrusion and the first mentioned protrusion cooperating with the bone plate adjacent the bone plate opening to align the one of said first bushing and said second bushing to the bone plate.

24. The guide as in claim 19:
wherein said tube includes a tube locating feature; and
wherein at least one of said first bushing and said second bushing includes a bushing locating feature, the bushing location feature cooperating with the tube locating feature to align said bushing in said tube.

25. The guide as in claim 19, further comprising an alignment fastener bushing fitted to said body, said alignment fastener bushing adapted to guide at least one of a alignment guide fastener drill fastener into cooperation with the bone and an alignment fastener into cooperation with the bone plate and the bone to assist in supporting the guide onto the bone.

26. The guide as in claim 19, wherein said body and said tube are adapted to provide progressive compression of the bone by the first mentioned fastener and a second fastener.

27. The guide as in claim 19, wherein said tube is integral with said body.

28. The guide as in claim 19:
wherein said guide is adapted for use with at least the first mentioned plate and a second plate, the first mentioned plate having a different shape than the second plate;
wherein said body comprises a riser and a first targeting guide; and
further comprising a second targeting guide, said second targeting guide and said first targeting guide cooperating selectively with said riser, said first targeting guide for cooperation with the first mentioned plate and said second targeting guide for cooperation with the second plate.

29. A method for repairing a bone fracture on a bone having a condylar portion and a shaft portion, the method including the steps of:
providing a bone plate having a head portion for cooperation with the condylar portion and a body portion for cooperation with the shaft portion and a first opening in the head portion and a second opening in the body portion;
providing a first fastener;
securing the head portion of the bone plate to the condylar portion of the bone with the first fastener;
providing a second fastener; and
securing the body portion of the bone plate to the shaft portion of the bone by percutaneously securing the second fastener to the body portion of the plate and to the shaft portion of the bone while urging the shaft portion of the bone toward the condylar portion of the bone.

30. The method of claim 29:
wherein the providing the bone plate further comprises providing the bone plate with a third opening in the body portion;
further comprising the step of providing a third fastener; and
further comprising the step of further compressing the bone by percutaneously securing the third fastener to the body portion of the plate and to the shaft portion of the bone while urging the shaft portion of the bone toward the condylar portion of the bone.

31. The method of claim 29, wherein the step of percutaneously securing the second fastener to the body portion of the plate and to the shaft portion of the bone includes the steps of:
providing a tube;
inserting the tube percutaneously through the skin to the bone plate; and
percutaneously securing the second fastener to the body through the tube.
